(19) United States
(12) Patent Application Publication
(10) Pub. No.: US 2024/0382591 A1
(43) Pub. Date: Nov. 21, 2024

(54) METHODS AND COMPOSITION USING PATIENT-DERIVED AUTOLOGOUS NEOANTIGENS FOR TREATING CANCER

(71) Applicants: THE UNIVERSITY OF CHICAGO, Chicago, IL (US); CHARITÉ - UNIVERSITÄTSMEDIZIN BERLIN, Berlin (DE)

(72) Inventors: Hans SCHREIBER, Chicago, IL (US); Matthias LEISEGANG, Berlin (DE); Steven Patrick WOLF, Chicago, IL (US); Vasiliki ANASTASOPOULOU, Berlin (DE); Karin SCHREIBER, Chicago, IL (US); Michael BISHOP, Chicago, IL (US); Amittha WICKREMA, Chicago, IL (US)

(73) Assignees: THE UNIVERSITY OF CHICAGO, Chicago, IL (US); CHARITÉ - UNIVERSITÄTSMEDIZIN BERLIN, Berlin (DE)

(21) Appl. No.: 18/694,134

(22) PCT Filed: Sep. 21, 2022
(86) PCT No.: PCT/US2022/076760

§ 371 Date: Mar. 21, 2024

Related U.S. Application Data
(60) Provisional application No. 63/246,633, filed on Sep. 21, 2021.

(51) Int. Cl. A61K 39/00; C07K 14/725; C07K 14/74; C12N 5/0781; C12N 5/0783; C12N 5/0784

(52) U.S. Cl. CPC A61K 39/464401; A61K 39/4611; A61K 39/4632; C07K 14/7051; C07K 14/70539; C12N 5/0635; C12N 5/0636; C12N 5/0639; C12N 2501/2304; C12N 2501/52

(57) ABSTRACT

The current disclosure provides for techniques and approaches for the generation of autologous mutant neoantigen-specific, TCR-engineered T cells used for adoptive transfer in treatment of cancer patients. Also provided are surrogate cancer cells, which is a personalized cell system that can be used for vaccination and TCR discovery in cancer patients.

### CROSS-REFERENCE TO RELATED APPLICATIONS

This application claims the benefit of priority of U.S. Provisional Patent Application No. 63/246,633 filed Sep. 21, 2021, which is hereby incorporated by reference in its entirety.

### GOVERNMENT SUPPORT STATEMENT

This invention was made with government support under grant number CA022677 awarded by the National Institutes of Health. The government has certain rights in the invention.

### BACKGROUND OF THE INVENTION

### I. Field of the Invention

This invention relates to the field of molecular biology and cancer therapy.

### II. Background

The selection of appropriate target structures, so-called antigens, on cancers is essential for cellular therapies using the patient's own T cells engineered with tumor-specific receptors. However, the tremendous diversity of cancer makes it impossible to identify tumor antigens that can serve as targets across different tumor entities and thus larger groups of patients. Approaches that break away from the short-sighted strategy of making T-cell therapy a treatment option that depends on pre-defined tumor antigens are needed, as each individual cancer comes with a set of the most desirable target structures—neoantigens. Neoantigens are a common characteristic of all cancers, as they are the result of somatic mutations, most of which were causative for tumor development. Therefore, methods that utilize a tumor's neoantigens as targets for vaccination and T cell therapy are needed in the art.

### SUMMARY OF THE INVENTION

The current disclosure provides for techniques and approaches for the generation of autologous mutant neoantigen-specific, TCR-engineered immune cells used for adoptive transfer in treatment of cancer patients. Also provided are surrogate cancer cells, which is a personalized cell system that can be used for vaccination and TCR discovery in cancer patients. Aspects of the disclosure relate to a B cell comprising an expression vector that encodes at least one minigene linked to a promoter and wherein the minigene encodes a neoantigen. Further aspects relate to a B cell comprising an expression vector that encodes at least one minigene linked to a promoter and wherein the minigene encodes a wild-type polypeptide that corresponds to a neoantigen. Also provided is a population of cells comprising at least two or more B cells of the disclosure, wherein each B cell comprises the same vector.

Further aspects relate to a method for generating a population of conditionally immortalized B cells comprising: isolating B cells from a subject; transferring a vector comprising at least one minigene linked to a promoter into the cells; wherein the minigene encodes a neoantigen; and contacting the cells with a composition comprising one or both of CD40 ligand (CD40L) and IL-4, thereby generating a population of conditionally immortalized B cells named surrogate cancer cell (SCC). Aspects also provide for a method for generating a population of immortalized B cells comprising: isolating B cells from a subject; transferring a vector comprising at least one minigene linked to a promoter into the cells; wherein the minigene encodes a wild-type polypeptide that corresponds to a neoantigen; and contacting the cells with a composition comprising one or both of CD40 ligand (CD40L) and IL-4, thereby generating a population of conditionally immortalized B cells named surrogate normal cells (SNC). In some aspects, the immortalized B cells are conditionally immortalized B cells. Also provided is a population of cells created by the methods of the disclosure. Aspects relate to a pool of 2 or more populations of B cells of the disclosure, wherein each population comprises a different vector.

Further aspects relate to a method for preparing a cell lysate comprising freezing and thawing B cells, a population of B cells, or a pool of B cells of the disclosure. Also provided is a cell lysate produced by the methods of the disclosure. Yet further aspects relate to a composition comprising B cells, population of B cells, pool of B cells, or a lysate of the disclosure. Methods also relate to a method for isolating and/or expanding neoantigen-specific immune cells from a subject comprising contacting in vitro a starting population of immune effector cells from the subject with a population of cells, pool of cells, cell lysate, or composition of the disclosure. Aspects also relate to a neoantigen-specific T cell, immune cell, or TCR produced according to methods of the disclosure. Also provided is a method for identifying cross-reactive engineered T cells comprising: contacting a population of subject-derived T cells from a subject with the B cells of the disclosure, or a cell lysate thereof, and evaluating the stimulation of the T cells after contact with the B cells. In some aspects, the method further comprises obtaining the subject-derived T cells from the subject.

Methods also relate to a method for treating or vaccinating a subject for cancer comprising administering a population of cells, pool of cells of claim, cell lysate, TCR, or composition of the disclosure. Further aspects relate to a method for treating or vaccinating a subject for cancer comprising: administering autologous engineered T cells to the subject, wherein the engineered T cells comprise: (i) CD8+ T cells that are stimulated in response to at least one MHIC class I restricted neoantigen; and (ii) CD4+ T cells that are stimulated in response to at least one MIC class II restricted neoantigen.

Methods also include methods of reducing tumor burden; methods of lysing a cancer cell; methods of killing tumor/cancerous cells; methods of increasing overall survival; methods of reducing the risk of getting cancer or of getting a tumor; methods of increasing recurrent free survival; methods of preventing cancer; and/or methods of reducing, eliminating, or decreasing the spread or metastasis of cancer, the method comprising administering a population of cells, pool of cells of claim, cell lysate, TCR, or composition of the disclosure to a subject in need thereof. Further methods include a method for treating a subject with a T cell receptor (TCR), the method comprising administering a TCR to the subject, wherein the TCR comprises a convergent CDR3-alpha and/or CDR3-beta. Also described is a method comprising: isolating TCRs from a subject; sequencing the isolated TCRs; and administering to the subject an engineered TCR comprising an alpha chain CDR1, CDR2, and CDR3 and a beta chain CDR1, CDR2, and CDR3 of a sequenced TCR; wherein the engineered TCR comprises a convergent CDR3-alpha and/or CDR3-beta.

The cell may be one that has been isolated from and/or derived from a subject having cancer. The neoantigen may be a neoantigen expressed in a cancer cell from the subject. neoantigen may comprise a non-synonymous single nucleotide variant (nsSNV) or frameshift mutation. The nsSNV or frameshift mutation may be in the center position of the minigene or in one of the center positions of the minigene. In some aspects, the nsSNV or frameshift mutation is +1, +2, +3, +4, +5, +6, +7, −1, −2, −3, −4, −5, −6, or −7 from the center position or positions of the minigene. The wild-type polypeptide may be the wild-type polypeptide that corresponds to a neoantigen comprising a nsSNV or frameshift mutation. The nucleotide corresponding to the nsSNV or frameshift mutation may be in the center position of the minigene or in one of the center positions of the minigene. In some aspects, the nucleotide corresponding to the nsSNV or frameshift mutation is +1, +2, +3, +4, +5, +6, +7, −1, −2, −3, −4, −5, −6, or −7 from the center position or positions of the minigene.

The B cell may comprise or further comprise an expression vector that encodes a non-immunogenic heterologous cell marker. The non-immunogenic heterologous cell marker may be CD11c. Examples of non-immunogenic heterologous cell markers useful in the methods and compositions of the disclosure include, but are not limited to, EpCAM, VEGFR, integrins (e.g. integrins αvβ, α4, α4β7, α5β1, αvβ), TNF receptor superfamily (e.g. TRAIL-R1, TRAIL-R2), PDGF receptor, interferon receptor, folate receptor, GPNMB, ICAM-1, CEA, CA-125, MUC1, TAG-72, IL-6 receptor, 5T4, GD2, or clusters of differentiation (e.g., CD2, CD3, CD4, CD5, CD11, CD11a/LFA-1, CD11c, CD15, CD18/ITGB2, CD23/IgE Receptor, CD25, CD28, CD30, CD33, CD38, CD41, CD44, CD51, CD62L, CD125, CD147/basigin, CD152/CTLA-4, CD195/CCR5, CD319/SLAMF7, and modifications and truncations thereof. The vector may encode at least two minigenes linked to a promoter. In some aspects, the vector encodes, encodes at least, or encodes at most 1, 2, 3, 4, 5, 6, 7, 8, 9, 10, 11, 12, 13, 14, 15, 16, 17, 18, 19, or 20 minigenes (or any derivable range therein) linked to a promoter, such as to a single promoter or to multiple promoters. In some aspects, the minigenes on one vector are linked to the same promoter. The vector may encode 2-15 minigenes linked to a promoter. In some aspects, the vector encodes 2, to 3, 4, 5, 6, 7, 8, 9, 10, 11, 12, 13, 14, 15, 16, 17, 18, 19, or 20 minigenes (or any derivable range therein) linked to a promoter. Each minigene may encode a different neoantigen. The neoantigen may comprise one or at least one nsSNV or frameshift mutation. The neoantigen may comprise, may comprise at least, or may comprise at most 1, 2, 3, 4, 5, 6, or 7 nsSNVs or frameshift mutations (or any derivable range therein). Each minigene may encode a different wild-type polypeptide that corresponds to a neoantigen.

The vector may encode or further encode one or more proteasomal cleavage sites between each minigene. The proteasomal cleavage site may comprise or consist of the amino acid sequence: AAY. In some aspects, the cell marker and the minigene(s) are encoded on the same vector. The cell marker and minigene(s) may be expressed from the same promoter. The vector may encode or further encode for a self-cleaving peptide between the minigene(s) and the cell marker. The self-cleaving peptide may be a 2A-element. The 2A cleavage site may comprise one or more of a P2A, F2A, E2A, or T2A cleavage site. Each minigene may also encode for a peptide having 10-30 amino acids in length. The minigene may encode for a peptide having, having at least, or having at most 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids, or any derivable range therein. In some aspects, each minigene is 25 amino acids in length. In some aspects, the expression vector is integrated into the genome of the B cell. In some aspects, the expression vector is extrachromosomal to the genome of the B cell. In some aspects, the expression vector is maintained as an extrachromosomal element (not integrated into the host genome). In some aspects, the B cell is a CD19+ cell.

In some aspects, the methods comprise or further comprise obtaining a starting population of immune effector cells from the subject. Methods of the disclosure may comprise or further comprise enriching the population of cells for the heterologous cell marker and/or for CD19. Enriching the population of cells for the heterologous cell marker and/or for CD19 may comprise or further comprise sorting the cells based on expression of the heterologous cell marker and/or CD19. In some aspects, the CD40L is provided from irradiated CD40L-positive cells. The CD40L may be a fragment of the CD40L that is capable of binding to CD40. The CD40L may be provided by providing CD40L positive cells or by providing the isolated CD40L polypeptide or CD40-binding fragment thereof. The composition may comprise 1 to 2 ng/mL or 10 to 30 U/mL IL-4. The composition may comprise, may comprise at least, or may comprise at most 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 ng/mL (or any derivable range therein) of IL-4. The composition may comprise, may comprise at least, or may comprise at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 U/mL (or any derivable range therein) of IL-4. In some aspects of the disclosure, the pool of cells comprises 2-6 populations. The pool may comprise, comprise at least, or comprise at most 2, 3, 4, 5, 6, 7, 8, 9, 10, 11, 12, 13, 14, 15, 16, 17, 18, 19, or 20 populations of B cells, or any derivable range therein. The B cells may be further defined as being conditionally immortal. The composition may comprise one or both of CD40 ligand (CD40L) and IL4. In some aspects, the composition comprises both of CD40L and IL4.

In the methods of the disclosure, the cells from the subject may be contacted in vitro with a cell lysate, pool of cells, or composition of the disclosure. The method may comprise or further comprise contacting the cells from the subject with antigen presenting cells (APCs), artificial antigen presenting cells (aAPCs), or an artificial antigen presenting surface (aAPSs). In some aspects, the APCs are dendritic cells. In some aspects, the APCs are peripheral blood derived dendritic cells. In some aspects, the APCs are conditionally immortalized B cells.

The cells from the subject may be from a biopsy specimen or tissue sample from the subject, or a fraction thereof. The biopsy or tissue sample may be one that comprises cancerous cells. The biopsy or tissue sample may be one that comprises tumor infiltrating lymphocytes (TILs). The cells from the subject may be from a peripheral blood sample from the subject.

The methods may comprise or further comprise generating a clonal population of neoantigen-specific immune effector cells by limiting or serial dilution followed by expansion of individual clones by a rapid expansion protocol. The methods may also comprise or further comprise generating a polyclonal population of cancer-specific immune effector cells by serial dilutions followed by expansion of polyclonal responders by a rapid expansion protocol. The method may comprise or further comprise analyzing stimulation of the cells from the subject after they have been contacted with the population of cells, pool of cells, or composition. In some aspects, analyzing stimulation of the cells comprises evaluating the interferon gamma (IFNg) production after contacting the cells from the subject in vitro with a population of cells, pool of cells, or cell lysate of the disclosure. The method may comprise or further comprise cloning of a T cell receptor (TCR) from the clonal or polyclonal population of neoantigen-specific immune effector cells. Cloning of the TCR may comprise cloning of a TCR alpha and a beta chain. The TCR may be identified using a 5′-Rapid amplification of cDNA ends (RACE) method or using single cell TCR-sequencing. In some aspects, the cloned TCR is subcloned into an expression vector. The expression vector may be a retroviral, non-viral, or lentiviral vector. In some aspects, the expression vector comprises a CRISPR/Cas expression system that allows for targeted integration of the TCR. The expression vector may also be a vector described herein. The expression vector may be transferred to the host cell to generate an engineered cell that expresses the TCR. The host cell may be transduced, transfected, or electroporated with the expression vector. The host cell may be an immune cell. The immune cell may be a T or B cell. The T cell may be a CD8+ T cell, CD4+ T cell, or T6 T cell.

The population of cells may be pooled with other populations to provide a mixed pool of cells that comprise more than one population. The mixed pool may be combined just before application or administration, such as at most or at least 1, 2, 3, 4, 5, 6, 7, 8, 9, 10, 11, 12, 13, 14, 15, 16, 17, 18, 19, 20, 21, 22, 23, 24, hours or 1, 1.5, 2, 2.5 days before administration or application.

In some aspects, the subject is one that has cancer. The cancer may be a stage I, II, III, or IV cancer. Stage I cancer refers to cancer that is localized to one part of the body. Stage II cancer refers to locally advanced cancer. Stage III: cancers are also locally advanced. Whether a cancer is designated as Stage II or Stage III may depend on the specific type of cancer; for example, in Hodgkin's Disease, Stage II indicates affected lymph nodes on only one side of the diaphragm, whereas Stage III indicates affected lymph nodes above and below the diaphragm. The specific criteria for Stages II and III with respect to specific cancers are known in the art. Stage IV cancer refers to cancer that has metastasized and spread to other organs or throughout the body. The cancer may be a cancer described herein or one known in the art. The population of cells or pool of cells may be autologous cells or the composition may comprise autologous cells. In some aspects, a population of CD8-positive and/or CD4-positive and neoantigen MHC tetramer-positive engineered immune cells are purified from the engineered host cells. In some aspects, a clonal population of neoantigen-specific engineered immune cells may be generated by limiting or serial dilution followed by expansion of individual clones by a rapid expansion protocol (REP). REP is a commonly used approach for T-cell expansion where T cells are expanded with IL-2, OKT-3, and irradiated allogeneic peripheral blood mononuclear cells (PBMCs) as feeder cells, including accessory cells expressing Fc-γ I receptor (FcγRI). The Fc-portion of immunoglobulin (Ig)G2a-subclass mouse antibodies, including the OKT-3 antibody, attach to FcγRI on human feeder cells. An anti-CD3 antibody bound to FcγRI induces a more optimal proliferation/differentiation signal to CD8+ T cell than anti-CD3/CD28 immobilized on a solid surface. This reflects the dual benefit of anti-CD3-T-cell receptor (TCR) crosslinking and the costimulation provided by cell-cell interaction between T cells and FcγRI+ accessory cells. The method may comprise or further comprise contacting the neo-antigen-specific immune cells or population of immune cells with a population of cells, pool of cells, cell lysate, or composition of the disclosure, wherein the minigene encodes a wild-type polypeptide that corresponds to a neoantigen. In some aspects, the method further comprises contacting the neo-antigen-specific immune cells or the population of immune cells with tumor cells or lysates thereof. The method may comprise or further comprise analyzing immune cell stimulation of the population of immune cells after they have been contacted with the population of cells, pool of cells, composition, tumor cells, or lysates thereof. Analyzing immune cell stimulation may comprise or further comprise evaluating the interferon gamma (IFNg) secretion of the cells. The population of immune cells may be a clonal or polyclonal population.

In some aspects, the population of immune cells or TCR-engineered cells of the disclosure may be contacted with cells or cell lysate comprising a minigene that encodes a wild-type polypeptide that corresponds to a neoantigen and wherein the immune cells were determined to be unstimulated. In some aspects, the population of immune cells or TCR-engineered cells are contacted with tumor cells or lysates thereof and wherein immune cells were determined to be stimulated.

In some aspects, the patient-derived immune or T cells and the B cells are derived from the same subject. Evaluating the stimulation of the T cells may comprise evaluating the level of IFNg production. The T cells may be determined to be cross-reactive when the T cells are evaluated as being stimulated after contact with the B cells. In some aspects, the T cells are determined to be non-cross-reactive when the T cells are evaluated as being unstimulated after contact with the B cells. The population of subject-derived T cells may comprise a clonal population. The population of subject-derived T cells may be an expanded population of TILs or peripheral blood T cells derived from the subject.

In the methods of the disclosure, the administered cells may be autologous cells. In some aspects, the administered cells are proliferation-incompetent. The administered cells may be irradiated cells. The subject may be one that has been diagnosed with cancer. In some aspects, the subject is a human subject. The methods may comprise or further comprise administering at least a second additional therapy. The additional therapy may be an anti-cancer agent. The additional therapy may also be one described herein. In some aspects, treating may comprise one or more of reducing tumor size; increasing the overall survival rate; reducing the risk of recurrence of the cancer; reducing the risk of progression; reducing metastasis, reducing the risk of metastasis, and/or increasing the chance of progression-free survival, relapse-free survival, and/or recurrence-free survival.

In methods of the disclosure, the CD8+ T cells may comprise a population of engineered T cells that are stimulated in response to one MHC class I restricted neoantigen; and/or the CD4+ T cells may comprise a population of engineered T cells that are stimulated in response to one MHC class II restricted neoantigen. The CD8+ and/or CD4+ T cells may be neoantigen-specific T cells generated from the methods described herein. In some aspects, the CD8+ and/or CD4+ T cells comprise a TCR generated from the methods described herein. The ratio of CD4+ to CD8+ may be 1:1. In some aspects, the ratio of CD4+ to CD8+ may be 1 to 1, 1.5, 2, 2.5, 3, 3.5, 4, 4.5, 5, 5.5, 6, 6.5, 7, 7.5, 8, 8.5, 9, 9.5, or 10 (or any derivable range therein). In some aspects, the ratio of CD8+ to CD4+ may be 1 to 1, 1.5, 2, 2.5, 3, 3.5, 4, 4.5, 5, 5.5, 6, 6.5, 7, 7.5, 8, 8.5, 9, 9.5, or 10 (or any derivable range therein). The CD4+ and CD8+ cells may be administered within 30 days of each other. In some aspects, the CD4+ and CD8+ cells are administered on the same day. In some aspects, the CD4+ and CD8+ cells are administered within 1, 2, 3, 4, 5, 6, 7, 8, 12, or 24 hours or within 1, 2, 3, 4, 5, or 6 days or within 1, 2, 3, 4, 5, 6, 7, 8, 9, 10, 11, 12, 13, or 14 weeks of each other (or any derivable range therein). In some aspects, the time between the administration of the CD4+ and of the CD8+ cells is, is at least, or is at most 1, 2, 3, 4, 5, 6, 7, 8, 12, or 24 hours, or 1, 2, 3, 4, 5, or 6 days, or 1, 2, 3, 4, 5, 6, 7, 8, 9, 10, 11, 12, 13, or 14 weeks (or any derivable range therein). In some aspects, the CD8+ T cells that are stimulated in response to at least one MHC class I restricted neoantigen comprise a TCR that has been isolated and cloned from T cells that are stimulated in response to subject-derived B cells comprising an expression vector that encodes at least one minigene linked to a promoter and wherein the minigene encodes a neoantigen. In some aspects, the CD4+ T cells that are stimulated in response to at least one MHC class II restricted neoantigen comprise a TCR that has been isolated and cloned from T cells that are stimulated in response to lysates of subject-derived B cells comprising an expression vector that encodes at least one minigene linked to a promoter and wherein the minigene encodes a neoantigen. In some aspects, about 1×10⁷ CD8+ T cells that are stimulated in response to at least one MHC class I restricted neoantigen and/or about 1×10⁷ CD4+ T cells that are stimulated in response to at least one MHC class II restricted neoantigen are administered to the subject.

In some aspects, the CD4+ and/or CD8+ T cells comprise a convergent CDR3-alpha and/or CDR3-beta. In some aspects, the T cells comprise a convergent CDR3-beta. In some aspects, the amino acid sequence of the alpha chain CDR1, CDR2, and CDR3 and beta chain CDR1, CDR2, and CDR3 of the administered TCR comprises the amino acid sequence of an alpha chain CDR1, CDR2, and CDR3 and beta chain CDR1, CDR2, and CDR3 from a TCR comprising a convergent CDR3-alpha and/or CDR3-beta that has been isolated and sequenced from cells from the subject. In some aspects, administering an engineered TCR comprises administering T cells comprising a heterologous nucleic acid encoding for the engineered TCR.

The T cells may further be defined as autologous T cells. In some aspects, the T cells are allogenic. The T cells may be CD4+ T cells, or CD8+ T cells. In some aspects, the T cells comprise CD4+ T cells. In some aspects, the T cells comprise CD8+ T cells. The convergent CDR3-alpha or convergent CDR3 beta may comprise an amino acid sequence that is identical in at least three different TCR clones isolated and sequenced from cells from the subject. In some aspects, the at least three different TCR clones comprise an identical CDR3-alpha and/or CDR3-beta amino acid sequence and a different nucleotide sequence. In some aspects, The convergent CDR3-alpha or convergent CDR3 beta may comprise an amino acid sequence that is identical in at least three, 4, 5, or 6 (or any derivable range therein) different TCR clones isolated and sequenced from cells from the subject. In some aspects, the nucleotide sequence differs at the V-J joint of the CDR3-alpha, the V-D joint of the CDR3-beta, and/or the D-J joint of the CDR3-beta. In some aspects, at least three different TCR clones comprise an identical CDR3-alpha and/or CDR3-beta amino acid and nucleotide sequence and wherein the V(D)J region haplotypes that are utilized to generate the CDR3 are different in the at least three different TCR clones. Sequencing the isolated TCRs may comprise single cell sequencing of nucleic acids isolated from T cells isolated from the subject. In some aspects, the TCR-beta chain is sequenced. In some aspects, the TCR-alpha chain is sequenced. In some aspects, the TCR-alpha and TCR-beta chain is sequenced. The T cells isolated from the subject may comprise CD3+, CD8+, and/or CD4+ T cells. In some aspects, the convergent CDR comprises a CDR with a frequency of greater than 0.01. In some aspects, the convergent CDR comprises a CDR with a frequency of, of at least, or of at most 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, or 0.5, or any range derivable therein. The frequency, when used with respect to CDR frequency, refers to the CDR frequency in the sequenced cells, expressed as a fraction or percentage (0.1=frequency of 1 in 10 or 10%). In some aspects, the CDR frequency is the frequency of a CDR having a particular amino acid sequence and wherein CDRs with the exact same nucleotide sequence are not included in the frequency determination.

The subject may be one that has been treated with a cancer vaccine. In some aspects, the subject has not been treated with a cancer vaccine. In some aspects, the subject has not previously been treated with an immunotherapy. The immunotherapy may comprise immune checkpoint blockade (ICB) therapy. In some aspects, the immunotherapy comprises adoptive T cell therapy, a tumor cell vaccine, or a dendritic cell vaccine.

The amount of cells administered to a subject may be, may be at least, or may be at most 1×10², 2×10², 3×10², 4×10², 5×10², 6×10², 7×10², 8×10², 9×10², 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1××10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, or 1×10¹⁴ cells (or any derivable range therein).

The subject may be a mammal. In some aspects, the subject comprises a laboratory test animal, such as a mouse, rat, rabbit, dog, cat, horse, or pig. In some aspects, the subject is a human.

The method may comprise or further comprise administering a cell or a composition comprising a cell and wherein the cell comprises an autologous cell. In some aspects, the cell comprises a non-autologous cell. The cell may also be allogenic or xenogenic.

“Treatment” or “Treating” may refer to any treatment of a disease in a mammal, including: (i) suppressing the disease, that is, causing the clinical symptoms of the disease not to develop by administration of a protective composition after the inductive event but prior to the clinical appearance or reappearance of the disease; (ii) inhibiting the disease, that is, arresting the development of clinical symptoms by administration of a protective composition after their initial appearance; and/or (iii) relieving the disease, that is, causing the regression of clinical symptoms by administration of a protective composition after their initial appearance. In some aspects, the treatment may exclude prevention of the disease.

CARE-TCR refers to a TCR that recognizes MHC class I restricted antigens on cancer cells. CIB refers to conditionally immortalized autologous B cells. CIBs can be a source for patient-derived autologous APCs. LYRE-TCR refers to a TCR that recognizes MHC class II restricted antigens that are processed and presented from lysates of cancer cells or tumor tissue. NeoAg refers to neoantigens, or antigens derived from nsSNVs that harbor neoepitopes that can be recognized by the adaptive immune system. nsSNV refers to non-synonymous single nucleotide variant or a single nucleotide substitution that leads to a mutated codon causing an amino acid exchange. SCC refers to surrogate cancer cell or a CIB that expresses mutant neoantigens. SNC refers to surrogate normal cell or a CIB that expresses normal self-antigens.

The subject may be a mammal. In some aspects, the subject comprises a laboratory test animal, such as a mouse, rat, rabbit, dog, cat, horse, or pig. In some aspects, the subject is a human.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.” Any term used in singular form also comprise plural form and vice versa.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other embodiments are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments and aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Identification of somatic mutations and generation of surrogate cancer cells (SCCs).

FIG. 2A-B. (A) Isolation of neoantigen-specific T cells from patient tumor-infiltrating lymphocytes. (B) Isolation of neoantigen-specific T cells from patient peripheral blood.

FIG. 3. Adoptive therapy using T cells engineered with neoantigen-specific TCRs.

FIG. 4A-C. Generation of surrogate cancer cells (SCCs). Illustration of (A) the generation of conditionally immortalized, CD40L-induced B cells as source for autologous APCs (CIBs) and (B) the identification of patient tumor-specific neoantigens used for construction of TMM-vectors. (C) Flow analysis of TMM-electroporated CIBs. Marker gene expression indicates successful generation of SCCs. The five different SCCs (SCC¹⁻¹⁰, SCC¹¹⁻²⁰, SCC²¹⁻³⁰, SCC³¹⁻⁴⁰ and SCC⁴¹⁻⁵⁰) represent CIBs electroporated with TMM¹⁻¹⁰, TMM¹¹⁻²⁰ TMM²¹⁻³⁰, TMM³¹⁻⁴⁰ or TMM⁴¹⁻⁵⁰ that encode for 10 different neoantigens linked to a marker gene.

FIG. 5. Identification of a CMV-specific CD8⁺ TCR isolated from one patient. Five different CD8⁺ TCRs were isolated from one patient and used to engineer CD8⁺ T cells. The TCR-engineered T cells were co-cultured for 24 h with CIBs from the same patient before supernatants were analyzed for IFN-7 concentrations by ELISA. The CIBs were electroporated with in vitro transcribed RNA of viral antigens. No stimulation or stimulation with water was used as negative control. Stimulation with ionomycin and phorbol myristate acetate (MAX) was used as TCR-independent control.

FIG. 6A-D. Therapeutic effective CD4⁺ TCRs used for generation of TCR-engineered CD4⁺ T cells recognize lysates generated either from tumor tissue or cancer cells. (A-D) CD4⁺ T cells were engineered with TCRs and used in in vitro assays or for adoptive T cell transfer against large and established tumors. The LYRE-TCR is specific for the mL9 mutant neoantigen expressed by 6132A cancer cells while the non-LYRE-TCR specifically recognizes the mL26 mutant neoantigen expressed by 6139B cancer cells. (A, C and D) Spleen cells from wild type C3H/HeN mice were used for T cell stimulation and co-cultured for 24 h before supernatants were analyzed for IFN-7 concentrations by ELISA. (A) Gradually, 10-fold diluted mL9 or mL26 peptide was used for T cell stimulation. (B) Either 6132A or 6139B tumor bearing C3H Rag2^{-/-} mice were treated 25 or 20 days after cancer cell inoculation as indicated by the arrow head. (C-D) Gradually, 3-fold diluted 6132A or 6139B (C) tumor tissue lysates or (D) cancer cell lysates were used for T cell stimulation.

FIG. 7A-D. One CARE-TCR and one LYRE-TCR are both essential and sufficient for effective tumor eradication by adoptive T cell transfer. (A-D) CD4⁺ and CD8⁺ T cells were TCR-engineered before used in in vitro assays or for adoptive T cell transfer against large and established tumors. (A-B) 6132A-specific CARE- and LYRE-TCR were used. (A) TCR-engineered T cells were co-cultured for 24 h with cancer cells or with lysates of cancer cells and spleen cells from the spleen of C3H/HeN mice before supernatants were analyzed for IFN-7 concentrations by ELISA. (B) 6132A tumor-bearing C3H Rag2^{-/-} mice were treated with the combination of one CARE-TCR CD8⁺ T cell population with one LYRE-TCR CD4⁺ T cell population around 25 days after cancer cell inoculation as indicated by the arrow head. All mice eradicated their tumors (n=11). (C-D) 6139B-specific CARE- and non-LYRE-TCR were used. (C) TCR-engineered T cells were co-cultured for 24 h with cancer cells or with lysates of cancer cells and spleen cells from the spleen of C3H/HeN mice before supernatants were analyzed for IFN-7 concentrations by ELISA. (D) 6139B tumor-bearing C3H Rag2^{-/-} mice were treated with the combination of one CARE-TCR CD8⁺ T cell population with one non-LYRE-TCR CD4⁺ T cell population around 22 days after cancer cell inoculation as indicated by the arrow head. All mice died due to tumor burden (n=4).

FIG. 8. Successive treatment using first LYRE-TCR CD4⁺ T cells and later CARE-CD8⁺ T cells for adoptive T cell transfer eradicates large and long-established solid tumors. 6132A tumor-bearing C3H Rag2^{-/-} mice were first treated with one LYRE-TCR CD4⁺ T cell population around 20 days after cancer cell inoculation as indicated by the black arrow head. Another 33 days later, mice received a second dose of a CARE-TCR CD8⁺ T cell population as indicated by the open arrow head. Both mice eradicated their tumors (n=2).

FIG. 9A-F. Independent T cell clones develop convergent TCRs against mL9. (A) 6132A tumor fragments were injected s.c. into C3H/HeN mice. Shown are six mice which developed tumors after fragment injection (55% (11/20) of injected C3H/HeN mice) and were used for TCR analysis. Results were compiled from three independent experiments. Grey dots indicate day of T cell analysis. (B) Shown is an example of T cells isolated from spleen and tumor sorted for life, CD3⁺, CD4⁺ and mL9-tetramer⁺ specificity. Indicated are percentages of mL9-tetramer positive T cells. CLIP-tetramer staining was used as negative control. (C) Frequencies of αmL9 TCR CDR3 amino acid sequences obtained from tumor and spleen of the six analyzed mice. (D) Amino acid CDR3 sequences of the α- and β-TCR chains of H6, H9, H12 and H13. (E) T cell clones showing genetic N-region diversity in the CDR3 α- and β-chains of the three TCRS H6, H9 and H13. (F) Frequency of the H6, H9 and H13 T cell clones among the six analyzed mice. Shown in FIG. 9D are SEQ ID NOS:23-30, respectively. The SEQ ID NOS of FIG. 9E are tabulated below:

| Clone | α-Chain | β-Chain |
|---|---|---|
| H6-1 | 31 | 32 |
| H6-2 | 33 | 34 |
| H6-3 | 31 | 35 |
| H6-4 | 31 | 36 |
| H6-5 | 39 | 34 |
| H6-6 | 31 | 37 |
| H6-7 | 31 | 38 |
| H9-1 | 40 | 41 |
| H9-2 | 40 | 42 |
| H9-3 | 40 | 43 |
| H9-4 | 40 | 44 |
| H9-5 | 40 | 45 |
| H9-6 | 40 | 46 |
| H13-1 | 47 | 49 |
| H13-2 | 48 | 50 |
| H13-3 | 48 | 51 |
| H13-4 | 48 | 49 |
| H13-5 | 48 | 52 |
| H13-6 | 48 | 53 |

FIG. 10A-E. mL9-specific TCR-engineered CD4⁺ T cells cause tumor destruction followed by growth arrest. (A) Outline of adoptive transfer using TCR-engineered T cells. (B-E) Spleen from C3H CD8^{-/-} mice were used as CD4⁺ T cell source for TCR-engineering. C3H Rag^{-/-} mice bearing 6132A tumors were treated with αmL9-TCR-engineered CD4⁺ T cells 21 to 25 days after cancer cell injection as indicated by the arrow head. (B) Top: Average tumor sizes were 0.558 cm³±0.122 cm³ standard deviation at day of treatment. Data are summarized from three independent experiments. (left) Treatment was performed with H6-T cells (n=6). (middle) Mice treated with αmL26-T cells, which are specific against an irrelevant antigen (n=4) have the same outcome as (right) untreated mice (n=4). Bottom: Average tumor sizes were 0.339 cm³±0.129 cm³ standard deviation at day of treatment. Data are summarized from two independent experiments. Treatment (n=5) was performed either with (left) H9- or (right) H12-CD4⁺ T cells. (C-E) 6132A-ECFP was used for injection into C3H Rag^{-/-} mice and tumors were left untreated (n=4) or treated with either H6- (n=4) or αmL26-T cells (n=4). Mice were injected with BrdU twice a day for three consecutive days before tumor tissue was isolated at day 20-25 after T cell transfer. (C) 6132A-ECFP cancer cells and TILs (CD3⁺, CD4⁺ and mL9-tetramer⁺) were analyzed by flow cytometry for frequency of BrdU incorporation. (D) 6132A-ECFP cancer cells and TAMs (CD11b⁺, F4/80⁺) were analyzed by flow cytometry for activation of cleaved caspase 3. (E) Significance between groups of 6132A cancer cells was determined by an ordinary one-way ANOVA with *P<0.05. Significance between groups of TILs was determined by a two-tailed Student's t-test with *P<0.05.

FIG. 11A-C. Stroma recognition is essential and sufficient for tumor destruction. (A) C3H Rag^{-/-} mice bearing 6132A MHC II KO tumors (red, n=8) were treated with H6-T cells 31 to 35 days after cancer cell injection, indicated by the arrow head. Spleen from C3H CD8^{-/-} mice were used as CD4⁺ T cell source for TCR-engineering. Average tumor sizes were 0.530 cm³±0.170 cm³ standard deviation at day of treatment. Data are summarized from two independent experiments. Shown are untreated tumors (black, n=2) as control. (B) Experimental design to determine whether stroma recognition is required for tumor destruction by αmL9-T cells. Mice received a BALB/c full thickness skin graft in addition to 6132A cancer cells. Splenocytes of TCR75-transgenic B6 Rag^{-/-} mice that express the TCR75 causing rejection of BALB/c skin graft when a K^{d}-derived epitope is presented on MHC class II I-A^{b} were engineered with the H6-TCR before used for adoptive T cell transfer. C3H mice possess the correct stroma to cause tumor destruction and growth arrest of 6132A. B6 mice possess the correct stroma to cause skin graft rejection of BALB/c skin. (C) Mice received a full thickness BALB/c skin graft on one flank and were injected s.c. with 6132A cancer cells on the other flank and received adoptive T cell transfer 21 to 25 days after cancer cell injection as indicated by the arrow head. (left) In one control experiment C3H Rag^{-/-} mice were used. Tumor size was 240 mm³ at day of therapy. The healthy BALB/c skin was maintained during the course of therapy. (right) B6 Rag^{-/-} mice were used (n=3) and are summarized from three independent experiments. Average tumor sizes were 324±136 mm³ at day of T cell transfer. Grey dots indicate the day on that more than 80% of the BALB/c skin graft was rejected.

FIG. 12A-C. mL9-specific CD4⁺ T cells induce NO expression in 6132A tumor-associated macrophages. (A) Spleen from C3H CD8^{-/-} mice were used as CD4⁺ T cell source for αmL9- or αmL26-TCR engineering. C3H Rag^{-/-} mice bearing 6132A tumors were treated with either H6- (n=3) or αmL26-T cells (n=3) 21 to 23 days after cancer cell injection. Tumor tissue was isolated at day 0, 6 or 20 to 22 after T cell transfer. Tumors were analyzed by FACS for frequency of life CD11b and F4/80⁺6132A tumor-associated macrophages (TAMs) expressing M1-type markers TNF, NO, IL-12 and I-E^{k} MHC class II at the different time points. (B) MHC class II I-E^{k} expressing TAMs were further analyzed by their frequency of expressing either Arginase, NO, both or none. (C) Frequency of NO and I-E^{k} expressing TAMs that were either positive or negative for arginase was determined from compiled day 6 and 20 data points and compared between αmL9- and αmL26-treated 6132A tumors. Significance between groups was determined by a two-tailed Student's t-test with **P≤0.01 and ***P≤0.001.

FIG. 13A-B. Progressively growing 6132A tumors are highly infiltrated by T cells. (A-B) 6132A tumors grown in C3H/HeN mice were analyzed by flow cytometry for CD3⁺ tumor infiltrating lymphocytes (left, all TILs), proportion of (middle) CD4⁺ TILs and (right) mL9-specific CD4⁺ TILs. (A) Shown is a representative example. (B) Results summarized from n=4 mice.

FIG. 14A-B. Non-specific T cell infiltration of 6132A tumors. (A-B) 6132A tumor-bearing C3H Rag^{-/-} mice were treated with TCR-engineered T cells. C3H CD8^{-/-} mice were used as CD4⁺ T cell source. Tumors were taken out 22 to 50 days after transfer of αmL9-TCR H6-T cells. When treated with αmL26-T cells, tumors were taken out 22 to 25 days after T cell transfer. (A) 6132A tumors were analyzed by flow cytometry for viable CD3⁺ and CD4+ tumor infiltrating lymphocytes (TILs) after αmL9-TCR H6 (n=5) or αmL26-T cell (n=3) transfer. (B) αCD3 immunohistochemistry (IHC) stain of tumor slides prepared 22 days after transfer of either (left) αmL9-TCR H6 or (right) αmL26 T cells.

FIG. 15A-E. Tumor vessel reduction upon transfer of mL9-specific CD4⁺ T cells. (A-E) Spleens from C3H CD8^{-/-} mice were transduced with the αmL9-H6 or the αmL26 TCR. (A) Example of longitudinal microscopy of tumor vessel reduction and cancer cell regression in 6132A-cerulean tumor bearing C3H Rag^{-/-} mice after transfer of H6-T cells. Tumor areas were randomly chosen before therapy and analyzed for (B) vessel and cancer cell reduction (n=6). DiD-labeled erythrocytes were used to visualize blood flow. Imaged area (in pixels) that was covered by vessels (black) or cancer cells from day 4 was set to 100%. Following days were assigned as percentage of maximum covered area. Indicated are an untreated control mouse (open circle) and the mouse (grey) shown in (A). (C-E) 6132A tumor-bearing mice were treated 25 days after cancer cell injection. Tumor tissue was analyzed on day 6, 7 and 8 after therapy by FACS. Control tumors received either no T cells (n=1) or αmL26-T cells (n=2) and were analyzed at day 8. Results are means±SD from two independent experiments. Significance between groups was determined by a two-tailed Student's t-test with *P≤0.05. (C) IFN-γ and (D) TNF concentrations were determined (n=8). (E) Tumors were analyzed for dead endothelial cells (Sytox-positive, CD146 and CD31 double-positive cell populations) (n=7).

FIG. 16A-B. Persistent detection of CD4⁺ T cells in tumor and periphery. (A-B) C3H CD8^{-/-} mice were used as CD4⁺ T cell source. 6132A tumor-bearing C3H Rag^{-/-} mice were treated with αmL9-TCR H6-engineered T cells. (A) (left) T cell transfer and analysis of peripheral blood are indicated by the arrow head. (right) The αmL9-specific H6-T cell population was detected by flow cytometry via α-Vβ6 and CD4 stain in peripheral blood several weeks after T cell transfer. Percentages are of Vβ6⁺ and CD4⁺ positive cells are indicated. Top: 43 days after T cell transfer. Shown is one out of three representative mice where blood was analyzed 40 to 50 days after T cell transfer. Bottom: 75 days after T cell transfer. Shown is one out of two representative mice where peripheral blood was analyzed 70 to 80 days after T cell transfer. (B) CD3 histology stain of 6132A tumor slides prepared (top) 89 and (bottom) 124 days after transfer of αmL9-TCR H6-T cells.

FIG. 17A-B. TCR-engineered T cells are mL9-specific and recognize stroma but not cancer cells directly. (A-B) TCR-engineered CD4⁺ T cells were co-cultured 24 h with indicated targets and supernatants were analyzed for IFN-γ concentrations by ELISA. Data are means±standard deviation and compiled from three independent experiments. (A) mL9-specific H6-CD4⁺ T cells were used for co-cultures. Top: (left) C3H/HeN spleen cells were cultured with various mL9 or mL26 (control) peptide concentrations. (right) 6132A cancer cells and lysates together with C3H/HeN spleen cells were used for co-culture. Cancer cells and lysates from 6139B, another C3H/HeN background cancer cell was used as control. Bottom: (left) 3-fold dilutions of cancer cell lysates were added to C3H/HeN spleen cells and used for co-culture. Cancer cell lysate from 6139B was used as control at the highest concentration. (right) Indicated numbers of CD11b and F4/80⁺ cells isolated from 6132A tumors grown in C3H Rag^{-/-} mice were used for co-culture. αmL26-CD4⁺ T cells were used as control and tested against the highest number of isolated cells. (B) Either mL9-specific (left) H9- or (right) H12-T cells were used for co-cultures with mutant or wild type L9 peptide cultured C3H/HeN spleen cells.

FIG. 18A-C. 6132A-TAMs show M2-type phenotype by expression of arginase. (A) Proportion of F4/80⁺ cells of bulk CD11b⁺ cells isolated from a representative 6132A tumor grown in C3H Rag^{-/-} mice analyzed by flow cytometry. (B-C) Spleen from C3H CD8^{-/-} mice were used as CD4⁺ T cell source for αmL9- or αmL26-TCR engineering. C3H Rag^{-/-} mice bearing 6132A tumors were treated 21 to 23 days after cancer cell injection. TAMs (CD11b⁺, F4/80*) were analyzed by flow cytometry. (B) Tumors were left untreated (n=4) or treated with either αmL9-TCR H6- (n=4) or αmL26-T cells (n=4). Tumor tissue was isolated at day 20-25 after T cell transfer. Frequency of dead TAMs is indicated. (C) Tumors were treated with either H6- (n=3) or αmL26-T cells (n=3) 21. Tumor tissue was isolated at day 0, 6 or 20-22 after T cell transfer. Tumor single cell suspensions were analyzed by FACS for frequency of TAMs expressing the M2-type markers TGFβ, Arginase and IL-10.

FIG. 19. Both, mL9-specific and mL26-specific TCR-engineered TILs show T-effector cell phenotype after adoptive T cell transfer. Spleen from C3H CD8^{-/-} mice were used as CD4⁺ T cell source for αmL9-TCR H6- or αmL26-TCR engineering. C3H Rag^{-/-} mice bearing 6132A tumors were treated 21 to 23 days after cancer cell injection. Tumors were isolated and TILs (viable, CD3⁺, CD4⁺ and either mL9- or mL26-tetramer⁺ T cells) were analyzed by flow cytometry for CD44 and CD62L expression. TILs from αmL26-treated tumors (n=2) were analyzed 20 to 22 days after T cell transfer. TILs from αmL9-treated tumors (n=4) were analyzed 80 to 93 days after T cell transfer.

### DETAILED DESCRIPTION OF THE INVENTION

The disclosure is based on evidence that demonstrates that T-cell therapy targeting neoantigens has the potential not only to delay tumor growth, but also to eradicate cancer in its entirety. This is achieved by selecting a suitable pair of T cell receptors (TCRs) that recognize neoantigens when presented in tumor tissue by patient MHC molecules. This approach requires that one of the two TCRs must recognize a neoantigen in the context of MHC class I and the other in the context of MHC class II. This approach, described in more detail in the examples, achieves the highest possible safety, as the patient's own T cells are used to identify therapeutic TCRs targeting neoantigens expressed uniquely in the patient's own tumor. This eliminates the risk of unpredictable cross-reactivity, which is frequently ignored or subsequently encountered when using TCRs from other sources. To increase the number of neoantigen-specific T cells in a patient prior to subsequent TCR isolation, the inventors developed a vaccination strategy using non-malignant surrogate cancer cells (SCCs) that are derived from non-malignant patient cells. SCCs will also be the central tool for the unbiased selection of optimal target structures from the entire pool of patient neoantigens and MHCs. The individual steps are incorporated into the inventors' proprietary approach that the inventors have named “Neo-T”. Neo-T allows the use of neoantigens of any given tumor as target structures for adoptively transferred T cells and provides a novel therapy design that is distinct from existing approaches. The unique elements of Neo-T open the door to a universal, personalized T-cell therapy that will facilitate a paradigm shift for the design of safe and efficient immunotherapies that are personalized in its literal sense

### I. LIBRARY CONSTRUCTION

### A. Vectors and Nucleic Acids

The current disclosure includes nucleic acids comprising one or more minigenes that represent neoantigens or the wild-type counterpart of a neoantigen from a subject with cancer. The terms “oligonucleotide:” “polynucleotide,” and “nucleic acid are used interchangeable and include linear oligomers of natural or modified monomers or linkages, including deoxyribonucleosides, ribonucleosides, α-anomeric forms thereof, peptide nucleic acids (PNAs), and the like, capable of specifically binding to a target (e.g. complementary or partially complementary) polynucleotide by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g. 3-4, to several tens of monomeric units. Whenever an oligonucleotide is represented by a sequence of letters, such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, unless otherwise noted. Analogs of phosphodiester linkages include phosphorothioate, phosphorodithioate, phosphoranilidate, phosphoramidate, and the like. It is clear to those skilled in the art when oligonucleotides having natural or non-natural nucleotides may be employed, e.g. where processing by enzymes is called for, usually oligonucleotides consisting of natural nucleotides are required.

The nucleic acid may be an “unmodified oligonucleotide” or “unmodified nucleic acid,” which refers generally to an oligomer or polymer of ribonucleic acid (RNA) or deoxyribonucleic acid (DNA). In some embodiments a nucleic acid molecule is an unmodified oligonucleotide. This term includes oligonucleotides composed of naturally occurring nucleobases, sugars and covalent internucleoside linkages. The term “oligonucleotide analog” refers to oligonucleotides that have one or more non-naturally occurring portions which function in a similar manner to oligonucleotides. Such non-naturally occurring oligonucleotides are often selected over naturally occurring forms because of desirable properties such as, for example, enhanced cellular uptake, enhanced affinity for other oligonucleotides or nucleic acid targets and increased stability in the presence of nucleases. The term “oligonucleotide” can be used to refer to unmodified oligonucleotides or oligonucleotide analogs.

Specific examples of nucleic acid molecules include nucleic acid molecules containing modified, i.e., non-naturally occurring internucleoside linkages. Such non-naturally internucleoside linkages are often selected over naturally occurring forms because of desirable properties such as, for example, enhanced cellular uptake, enhanced affinity for other oligonucleotides or nucleic acid targets and increased stability in the presence of nucleases. In a specific embodiment, the modification comprises a methyl group.

Nucleic acid molecules can have one or more modified internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides.

Modifications to nucleic acid molecules can include modifications wherein one or both terminal nucleotides is modified.

One suitable phosphorus-containing modified internucleoside linkage is the phosphorothioate internucleoside linkage. A number of other modified oligonucleotide backbones (internucleoside linkages) are known in the art and may be useful in the context of this embodiment.

Representative U.S. patents that teach the preparation of phosphorus-containing internucleoside linkages include, but are not limited to, U.S. Pat. Nos. 3,687,808; 4,469,863; 4,476,301; 5,023,243, 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 5,625,050, 5,489,677, and 5,602,240 each of which is herein incorporated by reference.

Modified oligonucleoside backbones (internucleoside linkages) that do not include a phosphorus atom therein have internucleoside linkages that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having amide backbones; and others, including those having mixed N, O, S and CH2 component parts.

Representative U.S. patents that teach the preparation of the above non-phosphorous-containing oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, each of which is herein incorporated by reference.

Oligomeric compounds can also include oligonucleotide mimetics. The term mimetic as it is applied to oligonucleotides is intended to include oligomeric compounds wherein only the furanose ring or both the furanose ring and the internucleotide linkage are replaced with novel groups, replacement of only the furanose ring with for example a morpholino ring, is also referred to in the art as being a sugar surrogate. The heterocyclic base moiety or a modified heterocyclic base moiety is maintained for hybridization with an appropriate target nucleic acid.

Oligonucleotide mimetics can include oligomeric compounds such as peptide nucleic acids (PNA) and cyclohexenyl nucleic acids (known as CeNA, see Wang et al., J. Am. Chem. Soc., 2000, 122, 8595-8602). Representative U.S. patents that teach the preparation of oligonucleotide mimetics include, but are not limited to, U.S. Pat. Nos. 5,539,082; 5,714,331; and 5,719,262, each of which is herein incorporated by reference. Another class of oligonucleotide mimetic is referred to as phosphonomonoester nucleic acid and incorporates a phosphorus group in the backbone. This class of olignucleotide mimetic is reported to have useful physical and biological and pharmacological properties in the areas of inhibiting gene expression (antisense oligonucleotides, ribozymes, sense oligonucleotides and triplex-forming oligonucleotides), as probes for the detection of nucleic acids and as auxiliaries for use in molecular biology. Another oligonucleotide mimetic has been reported wherein the furanosyl ring has been replaced by a cyclobutyl moiety.

Nucleic acid molecules can also contain one or more modified or substituted sugar moieties. The base moieties are maintained for hybridization with an appropriate nucleic acid target compound. Sugar modifications can impart nuclease stability, binding affinity or some other beneficial biological property to the oligomeric compounds.

Representative modified sugars include carbocyclic or acyclic sugars, sugars having substituent groups at one or more of their 2′, 3′ or 4′ positions, sugars having substituents in place of one or more hydrogen atoms of the sugar, and sugars having a linkage between any two other atoms in the sugar. A large number of sugar modifications are known in the art, sugars modified at the 2′ position and those which have a bridge between any 2 atoms of the sugar (such that the sugar is bicyclic) are particularly useful in this embodiment. Examples of sugar modifications useful in this embodiment include, but are not limited to compounds comprising a sugar substituent group selected from: OH; F; O-, S-, or N-alkyl; or O-alkyl-O-alkyl, wherein the alkyl, alkenyl and alkynyl may be substituted or unsubstituted C1 to C10 alkyl or C2 to C10 alkenyl and alkynyl. Particularly suitable are: 2-methoxyethoxy (also known as 2′-O-methoxyethyl, 2′-MOE, or 2′-OCH2CH2OCH3), 2′-O-methyl (2′-O—CH3), 2′-fluoro (2′-F), or bicyclic sugar modified nucleosides having a bridging group connecting the 4′ carbon atom to the 2′ carbon atom wherein example bridge groups include —CH2-O—(CH2)2-O— or —CH2-N(R3)-O wherein R3 is H or C1-C12 alkyl.

One modification that imparts increased nuclease resistance and a very high binding affinity to nucleotides is the 2′-MOE side chain (Baker et al., J. Biol. Chem., 1997, 272, 11944-12000). One of the immediate advantages of the 2′-MOE substitution is the improvement in binding affinity, which is greater than many similar 2′ modifications such as O-methyl, O-propyl, and O-aminopropyl. Oligonucleotides having the 2′-MOE substituent also have been shown to be antisense inhibitors of gene expression with promising features for in vivo use (Martin, P., Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926).

2′-Sugar substituent groups may be in the arabino (up) position or ribo (down) position. One 2′-arabino modification is 2′-F. Similar modifications can also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, each of which is herein incorporated by reference in its entirety.

Nucleic acid molecules can also contain one or more nucleobase (often referred to in the art simply as “base”) modifications or substitutions which are structurally distinguishable from, yet functionally interchangeable with, naturally occurring or synthetic unmodified nucleobases. Such nucleobase modifications can impart nuclease stability, binding affinity or some other beneficial biological property to the oligomeric compounds. As used herein, “unmodified” or “natural” nucleobases include the purine bases adenine (A) and guanine (G), and the pyrimidine bases thymine (T), cytosine (C) and uracil (U). Modified nucleobases also referred to herein as heterocyclic base moieties include other synthetic and natural nucleobases, many examples of which such as 5-methylcytosine (5-me-C), 5-hydroxymethyl cytosine, 7-deazaguanine and 7-deazaadenine among others.

Heterocyclic base moieties can also include those in which the purine or pyrimidine base is replaced with other heterocycles, for example 7-deaza-adenine, 7-deazaguanosine, 2-aminopyridine and 2-pyridone. Some nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds. These include 5-substituted pyrimidines, 6-azapyrimidines and N-2, N-6 and 0-6 substituted purines, including 2 aminopropyladenine, 5-propynyluracil and 5-propynylcytosine.

Additional modifications to nucleic acid molecules are disclosed in U.S. Patent Publication 2009/0221685, which is hereby incorporated by reference. Also disclosed herein are additional suitable conjugates to the nucleic acid molecules.

The term “vector” is used to refer to a carrier nucleic acid molecule into which a heterologous nucleic acid sequence can be inserted for introduction into a cell where it can be replicated and expressed and/or integrated into the host cell's genome. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Vectors include DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al., 2001; Ausubel et al., 1996, both incorporated herein by reference). Vectors may be used in a host cell to produce an antibody.

The term “expression vector” refers to a vector containing a nucleic acid sequence coding for at least part of a gene product capable of being transcribed or stably integrate into a host cell's genome and subsequently be transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described herein.

The vectors disclosed herein can be any nucleic acid vector known in the art. Exemplary vectors include plasmids, cosmids, bacterial artificial chromosomes (BACs) and viral vectors as well as CRISPR/Cas based systems.

Any expression vector for animal cell can be used. Examples of suitable vectors include pAGE107 (Miyaji et al., 1990), pAGE103 (Mizukami and Itoh, 1987), pHSG274 (Brady et al., 1984), pKCR (O'Hare et al., 1981), pSG1 beta d2-4 (Miyaji et al., 1990) and the like.

Other examples of plasmids include replicating plasmids comprising an origin of replication, or integrative plasmids, such as for instance pUC, pcDNA, pBR, and the like.

Other examples of viral vectors include adenoviral, lentiviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

Examples of promoters and enhancers used in the expression vector for animal cell include early promoter and enhancer of SV40 (Mizukami and Itoh, 1987), LTR promoter and enhancer of Moloney mouse leukemia virus (Kuwana et al., 1987), murine myeloproliferative sarcoma virus promoter (MPSV, Baum et al. 1995), eukaryotic translation elongation factor 1 alpha promoter (EF-1 alpha), promoter (Mason et al., 1985) and enhancer (Gillies et al., 1983) of immunoglobulin H chain and the like.

A specific initiation signal also may be required for efficient translation of coding sequences. These signals include the ATG initiation codon or adjacent sequences such as the Kozak sequence. Exogenous translational control signals, including the ATG initiation codon, may need to be provided. One of ordinary skill in the art would readily be capable of determining this and providing the necessary signals.

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.)

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997, incorporated herein by reference.) In aspects of the disclosure, condon-optimized vectors and nucleic acids are contemplated.

The vectors or constructs will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels. In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to be more stable and are translated more efficiently. Thus, in other embodiments involving eukaryotes, it is preferred that that terminator comprises a signal for the cleavage of the RNA, and it is more preferred that the terminator signal promotes polyadenylation of the message.

In expression, particularly eukaryotic expression, one will typically include a polyadenylation signal to effect proper polyadenylation of the transcript.

In order to propagate a vector in a host cell, it may contain one or more origins of replication sites (often termed “ori”), which is a specific nucleic acid sequence at which replication is initiated. Alternatively, an autonomously replicating sequence (ARS) can be employed if the host cell is yeast.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

A further aspect of the disclosure relates to a cell or cells. In some embodiments, a prokaryotic or eukaryotic cell is genetically transformed or transfected with at least one nucleic acid molecule or vector according to the disclosure. In some embodiments, the cells are infected with a viral particle of the current disclosure. In some embodiments, the cells are transfected with plasmids/vectors by electroporation.

The term “transformation” or “transfection” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed” or “transfected.” The construction of expression vectors in accordance with the current disclosure, and the transformation or transfection of the host cells can be carried out using conventional molecular biology techniques.

Suitable methods for nucleic acid delivery for transformation/transfection of a cell, a tissue or an organism for use with the current invention are believed to include virtually any method by which a nucleic acid (e.g., DNA) can be introduced into a cell, a tissue or an organism, as described herein or as would be known to one of ordinary skill in the art (e.g., Stadtfeld and Hochedlinger, Nature Methods 6(5):329-330 (2009); Yusa et al., Nat. Methods 6:363-369 (2009); Woltjen et al., Nature 458, 766-770 (9 Apr. 2009)). Such methods include, but are not limited to, direct delivery of DNA such as by ex vivo transfection (Wilson et al., Science, 244:1344-1346, 1989, Nabel and Baltimore, Nature 326:711-713, 1987), optionally with Fugene6 (Roche) or Lipofectamine (Invitrogen), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, J. Cell Biol., 101:1094-1099, 1985; U.S. Pat. No. 5,789,215, incorporated herein by reference); by electroporation (U.S. Pat. No. 5,384,253, incorporated herein by reference; Tur-Kaspa et al., Mol. Cell Biol., 6:716-718, 1986; Potter et al., Proc. Nat'l Acad. Sci. USA, 81:7161-7165, 1984); by calcium phosphate precipitation (Graham and Van Der Eb, Virology, 52:456-467, 1973; Chen and Okayama, Mol. Cell Biol., 7(8):2745-2752, 1987; Rippe et al., Mol. Cell Biol., 10:689-695, 1990); by using DEAE-dextran followed by polyethylene glycol (Gopal, Mol. Cell Biol., 5:1188-1190, 1985); by direct sonic loading (Fechheimer et al., Proc. Nat'l Acad. Sci. USA, 84:8463-8467, 1987); by liposome mediated transfection (Nicolau and Sene, Biochim. Biophys. Acta, 721:185-190, 1982; Fraley et al., Proc. Nat'l Acad. Sci. USA, 76:3348-3352, 1979; Nicolau et al., Methods Enzymol., 149:157-176, 1987; Wong et al., Gene, 10:87-94, 1980; Kaneda et al., Science, 243:375-378, 1989; Kato et al., J Biol. Chem., 266:3361-3364, 1991) and receptor-mediated transfection (Wu and Wu, Biochemistry, 27:887-892, 1988; Wu and Wu, J. Biol. Chem., 262:4429-4432, 1987); and any combination of such methods, each of which is incorporated herein by reference

### B. Detection, Selection, and Sequencing Elements of the Nucleic Acids.

The nucleic acids of the disclosure comprising the minigenes or nucleic acids encoding polypeptides or TCRs of the disclosure may comprise one or more polynucleotide sequences encoding for one or more protein or peptide tag, screenable gene, selectable gene, and/or barcode.

### 1. Selection and Screening Genes

In certain embodiments of the disclosure, the nucleic acids, such as the vectors described herein, may comprise or further comprise a selection or screening gene. Furthermore, the cells of the disclosure may further comprise a selection or screening gene. Such genes would confer an identifiable change to the cell permitting easy identification of cells that have the minigenes or polypeptides of the disclosure. Generally, a selectable (i.e. selection gene) gene is one that confers a property that allows for selection. A positive selectable gene is one in which the presence of the gene or gene product allows for its selection, while a negative selectable gene is one in which its presence of the gene or gene product prevents its selection. An example of a positive selectable gene is an antibiotic resistance gene.

Usually the inclusion of a drug selection gene aids in the cloning and identification of cells that have an activated receptor gene through, for example, successful ligand engagement. The selection gene may be a gene that confers resistance to neomycin, puromycin, hygromycin, DHFR, GPT, zeocin, G418, phleomycin, blasticidin, and histidinol, for example. In addition to genes conferring a phenotype that allows for the discrimination of receptor activation based on the implementation of conditions, other types of genes, including screenable genes such as GFP, whose gene product provides for colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ screenable genes and their protein products, possibly in conjunction with FACS analysis. Further examples of selectable and screenable genes are well known to one of skill in the art. In certain embodiments, the gene produces a fluorescent protein, an enzymatically active protein, a luminescent protein, a photoactivatable protein, a photoconvertible protein, or a colorimetric protein. Fluorescent markers include, for example, GFP and variants such as YFP, RFP etc., and other fluorescent proteins such as DsRed, mPlum, mCherry, YPet, Emerald, CyPet, T-Sapphire, Luciferase, and Venus. Photoactivatable markers include, for example, KFP, PA-mRFP, and Dronpa. Photoconvertible markers include, for example, mEosFP, KikGR, and PS-CFP2. Luminescent proteins include, for example, Neptune, FP595, and phialidin.

### 2. Protein or Peptide Tags

Exemplary protein/peptide tags include AviTag, a peptide allowing biotinylation by the enzyme BirA and so the protein can be isolated by streptavidin (GLNDIFEAQKIEWHE—SEQ ID NO:2), Calmodulin-tag, a peptide bound by the protein calmodulin (KRRWKKNFIAVSAANRFKKISSSGAL—SEQ ID NO:3), polyglutamate tag, a peptide binding efficiently to anion-exchange resin such as Mono-Q (EEEEEE—SEQ ID NO:4), E-tag, a peptide recognized by an antibody (GAPVPYPDPLEPR—SEQ ID NO:5), FLAG-tag, a peptide recognized by an antibody (DYKDDDDK—SEQ ID NO:6), HA-tag, a peptide from hemagglutinin recognized by an antibody (YPYDVPDYA—SEQ ID NO:7), His-tag, 5-10 histidines bound by a nickel or cobalt chelate (HHIHH—SEQ ID NO:8), Myc-tag, a peptide derived from c-myc recognized by an antibody (EQKLISEEDL—SEQ ID NO:9), NE-tag, a novel 18-amino-acid synthetic peptide (TKENPRSNQEESYDDNES—SEQ ID NO:10) recognized by a monoclonal IgGI antibody, which is useful in a wide spectrum of applications including Western blotting, ELISA, flow cytometry, immunocytochemistry, immunoprecipitation, and affinity purification of recombinant proteins, S-tag, a peptide derived from Ribonuclease A (KETAAAKFERQHMDS—SEQ ID NO:11), SBP-tag, a peptide which binds to streptavidin (MDEKTTGWRGGHVVEGLAGELEQLRARLEHHPQGQREP—SEQ ID NO:12), Softag 1, for mammalian expression (SLAELLNAGLGGS—SEQ ID NO:13), Softag 3, for prokaryotic expression (TQDPSRVG—SEQ ID NO:14), Strep-tag, a peptide which binds to streptavidin or the modified streptavidin called streptactin (Strep-tag II: WSHPQFEK—SEQ ID NO:15), TC tag, a tetracysteine tag that is recognized by FlAsH and ReAsH biarsenical compounds (CCPGCC—SEQ ID NO:16), V5 tag, a peptide recognized by an antibody (GKPIPNPLLGLDST—SEQ ID NO:17), VSV-tag, a peptide recognized by an antibody (YTDIEMNRLGK—SEQ ID NO:18), Xpress tag (DLYDDDDK—SEQ ID NO:19), Covalent peptide tags, Isopeptag, a peptide which binds covalently to pilin-C protein (TDKDMTITFTNKKDAE—SEQ ID NO:21), SpyTag, a peptide which binds covalently to SpyCatcher protein (AHIVMVDAYKPTK—SEQ ID NO:22), SnoopTag, a peptide which binds covalently to SnoopCatcher protein (KLGDIEFIKVNK—SEQ ID NO:20), BCCP (Biotin Carboxyl Carrier Protein), a protein domain biotinylated by BirA enabling recognition by streptavidin, Glutathione-S-transferase-tag, a protein which binds to immobilized glutathione, Green fluorescent protein-tag, a protein which is spontaneously fluorescent and can be bound by nanobodies, HaloTag, a mutated bacterial haloalkane dehalogenase that covalently attaches to a reactive haloalkane substrate, this allows attachment to a wide variety of substrates, Maltose binding protein-tag, a protein which binds to amylose agarose, Nus-tag, Thioredoxin-tag, Fc-tag, derived from immunoglobulin Fc domain, allow dimerization and solubilization. Can be used for purification on Protein-A Sepharose, Designed Intrinsically Disordered tags containing disorder promoting amino acids (P,E,S,T,A,Q,G, . . . ), and Ty-tag

### C. Barcodes

The nucleic acids of the disclosure may comprise or further comprise a barcode region that can identify the minigene, neoantigen, or wildtype counterpart thereof. The barcode region can be a polynucleotide of at least, at most, or exactly 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200 or more (or any range derivable therein) nucleotides in length. The barcode may comprise or further comprise one or more universal PCR regions, adaptors, linkers, or a combination thereof.

Methods of the disclosure may include determining the identity of the barcode by determining the nucleotide sequence of the index region in order to identify which receptor(s) has been activated in a population of cells. As discussed herein, methods may involve sequencing one nucleic acid region, such as a minigene, neoantigen, wild-type counterpart of a neoantigen, or TCR gene.

Nucleic acid constructs are generated by any means known in the art, including through the use of polymerases and solid state nucleic acid synthesis (e.g., on a column, multiwall plate, or microarray). The barcodes may correspond to a unique minigene or a group of minigenes, such as to at least, at most, or exactly 1, 2, 3, 4, 5, 6, 7, 8, 9, 10, 11, 12, 13, 14, 15, 16, 17, 18, 19, or 20 minigenes (or any derivable range therein). These barcodes may be oriented in the expression vector such that they are transcribed in the same mRNA transcript as the open reading frame of the minigenes or other gene (such as a TCR gene). The barcodes may be oriented in the mRNA transcript 5′ to the open reading frame, 3′ to the open reading frame, immediately 5′ to the terminal poly-A tail, or somewhere in-between. In some embodiments, the barcodes are in the 3′ untranslated region.

The unique portions of the barcodes may be continuous along the length of the barcode sequence or the barcode may include stretches of nucleic acid sequence that is not unique to any one barcode. In one application, the unique portions of the barcodes may be separated by a stretch of nucleic acids that is removed by the cellular machinery during transcription into mRNA (e.g., an intron).

The barcodes and/or index regions are quantified or determined by methods known in the art, including quantitative sequencing (e.g., using an Illumina® sequencer) or quantitative hybridization techniques (e.g., microarray hybridization technology or using a Luminex® bead system). Sequencing methods are further described herein.

### D. Sequencing Methods

Methods of the disclosure may include sequencing of the minigenes, vectors, and/or TCR genes described herein. Sequencing methods are known in the art and also described below.

### 1. Massively Parallel Signature Sequencing (MPSS).

The first of the next-generation sequencing technologies, massively parallel signature sequencing (or MPSS), was developed in the 1990s at Lynx Therapeutics. MPSS was a bead-based method that used a complex approach of adapter ligation followed by adapter decoding, reading the sequence in increments of four nucleotides. This method made it susceptible to sequence-specific bias or loss of specific sequences. Because the technology was so complex, MPSS was only performed ‘in-house’ by Lynx Therapeutics and no DNA sequencing machines were sold to independent laboratories. Lynx Therapeutics merged with Solexa (later acquired by Illumina) in 2004, leading to the development of sequencing-by-synthesis, a simpler approach acquired from Manteia Predictive Medicine, which rendered MPSS obsolete. However, the essential properties of the MPSS output were typical of later “next-generation” data types, including hundreds of thousands of short DNA sequences. In the case of MPSS, these were typically used for sequencing cDNA for measurements of gene expression levels. Indeed, the powerful Illumina HiSeq2000, HiSeq2500 and MiSeq systems are based on MPSS.

### 2. Polony Sequencing.

The Polony sequencing method, developed in the laboratory of George M. Church at Harvard, was among the first next-generation sequencing systems and was used to sequence a full genome in 2005. It combined an in vitro paired-tag library with emulsion PCR, an automated microscope, and ligation-based sequencing chemistry to sequence an E. coli genome at an accuracy of >99.9999% and a cost approximately 1/9 that of Sanger sequencing. The technology was licensed to Agencourt Biosciences, subsequently spun out into Agencourt Personal Genomics, and eventually incorporated into the Applied Biosystems SOLiD platform, which is now owned by Life Technologies.

### 3. 454 Pyrosequencing.

A parallelized version of pyrosequencing was developed by 454 Life Sciences, which has since been acquired by Roche Diagnostics. The method amplifies DNA inside water droplets in an oil solution (emulsion PCR), with each droplet containing a single DNA template attached to a single primer-coated bead that then forms a clonal colony. The sequencing machine contains many picoliter-volume wells each containing a single bead and sequencing enzymes. Pyrosequencing uses luciferase to generate light for detection of the individual nucleotides added to the nascent DNA, and the combined data are used to generate sequence read-outs. This technology provides intermediate read length and price per base compared to Sanger sequencing on one end and Solexa and SOLiD on the other.

### 4. Illumina (Solexa) Sequencing.

Solexa, now part of Illumina, developed a sequencing method based on reversible dye-terminators technology, and engineered polymerases, that it developed internally. The terminated chemistry was developed internally at Solexa and the concept of the Solexa system was invented by Balasubramanian and Klennerman from Cambridge University's chemistry department. In 2004, Solexa acquired the company Manteia Predictive Medicine in order to gain a massivelly parallel sequencing technology based on “DNA Clusters”, which involves the clonal amplification of DNA on a surface. The cluster technology was co-acquired with Lynx Therapeutics of California. Solexa Ltd. later merged with Lynx to form Solexa Inc.

In this method, DNA molecules and primers are first attached on a slide and amplified with polymerase so that local clonal DNA colonies, later coined “DNA clusters”, are formed. To determine the sequence, four types of reversible terminator bases (RT-bases) are added and non-incorporated nucleotides are washed away. A camera takes images of the fluorescently labeled nucleotides, then the dye, along with the terminal 3′ blocker, is chemically removed from the DNA, allowing for the next cycle to begin. Unlike pyrosequencing, the DNA chains are extended one nucleotide at a time and image acquisition can be performed at a delayed moment, allowing for very large arrays of DNA colonies to be captured by sequential images taken from a single camera.

Decoupling the enzymatic reaction and the image capture allows for optimal throughput and theoretically unlimited sequencing capacity. With an optimal configuration, the ultimately reachable instrument throughput is thus dictated solely by the analog-to-digital conversion rate of the camera, multiplied by the number of cameras and divided by the number of pixels per DNA colony required for visualizing them optimally (approximately 10 pixels/colony). In 2012, with cameras operating at more than 10 MHz A/D conversion rates and available optics, fluidics and enzymatics, throughput can be multiples of 1 million nucleotides/second, corresponding roughly to one human genome equivalent at 1× coverage per hour per instrument, and one human genome re-sequenced (at approx. 30×) per day per instrument (equipped with a single camera).

### 5. SOLiD Sequencing.

Applied Biosystems' (now a Life Technologies brand) SOLiD technology employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting beads, each containing single copies of the same DNA molecule, are deposited on a glass slide. The result is sequences of quantities and lengths comparable to Illumina sequencing. This sequencing by ligation method has been reported to have some issue sequencing palindromic sequences.

6. Ion Torrent semiconductor sequencing.

Ion Torrent Systems Inc. (now owned by Life Technologies) developed a system based on using standard sequencing chemistry, but with a novel, semiconductor based detection system. This method of sequencing is based on the detection of hydrogen ions that are released during the polymerization of DNA, as opposed to the optical methods used in other sequencing systems. A microwell containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

### 7. DNA Nanoball Sequencing.

DNA nanoball sequencing is a type of high throughput sequencing technology used to determine the entire genomic sequence of an organism. The company Complete Genomics uses this technology to sequence samples submitted by independent researchers. The method uses rolling circle replication to amplify small fragments of genomic DNA into DNA nanoballs. Unchained sequencing by ligation is then used to determine the nucleotide sequence. This method of DNA sequencing allows large numbers of DNA nanoballs to be sequenced per run and at low reagent costs compared to other next generation sequencing platforms. However, only short sequences of DNA are determined from each DNA nanoball which makes mapping the short reads to a reference genome difficult. This technology has been used for multiple genome sequencing projects and is scheduled to be used for more.

### 8. Heliscope Single Molecule Sequencing.

Heliscope sequencing is a method of single-molecule sequencing developed by Helicos Biosciences. It uses DNA fragments with added poly-A tail adapters which are attached to the flow cell surface. The next steps involve extension-based sequencing with cyclic washes of the flow cell with fluorescently labeled nucleotides (one nucleotide type at a time, as with the Sanger method). The reads are performed by the Heliscope sequencer. The reads are short, up to 55 bases per run, but recent improvements allow for more accurate reads of stretches of one type of nucleotides. This sequencing method and equipment were used to sequence the genome of the M13 bacteriophage.

### 9. Single Molecule Real Time (SMRT) Sequencing.

SMRT sequencing is based on the sequencing by synthesis approach. The DNA is synthesized in zero-mode wave-guides (ZMWs)—small well-like containers with the capturing tools located at the bottom of the well. The sequencing is performed with use of unmodified polymerase (attached to the ZMW bottom) and fluorescently labelled nucleotides flowing freely in the solution. The wells are constructed in a way that only the fluorescence occurring by the bottom of the well is detected. The fluorescent label is detached from the nucleotide at its incorporation into the DNA strand, leaving an unmodified DNA strand. According to Pacific Biosciences, the SMRT technology developer, this methodology allows detection of nucleotide modifications (such as cytosine methylation). This happens through the observation of polymerase kinetics. This approach allows reads of 20,000 nucleotides or more, with average read lengths of 5 kilobases.

### II. CD40 LIGAND

Embodiments of the disclosure may include the addition of CD40 ligand (CD40L) or cells comprising CD40L. CD40L, also called CD154 or CD40 ligand, is a protein that is primarily expressed on activated T cells and is a member of the TNF superfamily of molecules. The CD40L may be the entire protein or a fragment thereof, such as a CD40-binding fragment. The structure of CD40L and its interaction with CD40 is known in the art (see, for example, Schonbeck U, Libby P (January 2001). “The CD40/CD154 receptor/ligand dyad”. Cellular and Molecular Life Sciences. 58 (1): 4-43, which is hereby incorporated by reference). The CD40L of the disclosure may be a CD40L polypeptide or fragment that has a certain function, such as a polypeptide or fragment that binds to CD40, α5β1 integrin, and/or αIIbβ3. The CD40L polypeptide or fragment may provide costimulation. The CD40L polypeptide or fragment may promote B cell maturation and function, for example, by engaging CD40 on the B cell surface and therefore facilitating cell-cell communication. The CD40L may be embedded in the membrane of a cell. In some aspects, the CD40L is in soluble form.

An exemplary sequence of CD40L is: MIETYNQTSPRSAATGLPISMKIFMYLLTVFLITQMIGSALFAVYLHRRLDKIEDERNL HEDFVFMKTIQRCNTGERSLSLLNCEEIKSQFEGFVKDIMLNKEETKKENSFEMQKG DQNPQIAAHVISEASSKTTSVLQWAEKGYYTMSNNLVTLENGKQLTVKRQGLYYIY AQVTFCSNREASSQAPFIASLCLKSPGRFERILLRAANTHSSAKPCGQQSIHLGGVFEL QPGASVFVNVTDPSQVSHGTGFTSFGLLKL (SEQ ID NO:1). The CD40L may be a polypeptide comprising SEQ ID NO:1 or a fragment thereof.

The CD40L may be a polypeptide or polypeptide fragment starting at amino acid 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, or 260 of SEQ ID NO:1 and ending at amino acid 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, or 261 of SEQ ID NO:1. The polypeptide or fragment may comprise, may comprise at most, or may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, or any derivable range therein, contiguous amino acids of SEQ ID NO:1. The polypeptide or fragment may comprise, may comprise at least, or may comprise at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:1.

The CD40L polypeptide or fragment may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 substitutions (or any range derivable therein) relative to SEQ ID NO:1.

The substitution may be at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, or 261 of SEQ ID NO:1 and may be a substitution with any amino acid or may be a substitution with a alanine, arginine, asparagine, aspartic acid, cysteine, glutamine, glutamic acid, glycine, histidine, isoleucine, leusine, lysine, methionine, phenylalanine, proline, serine, threonine, tryptophan, tyrosine, or valine.

In some aspects, the CD40L polypeptide or fragment may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, or 261 (or any derivable range therein) contiguous amino acids of SEQ ID NO:1 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to SEQ ID NO:1.

### III. SAMPLE PREPARATION

In certain aspects, methods involve obtaining a sample from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain embodiments the sample is obtained from a biopsy from esophageal tissue by any of the biopsy methods previously mentioned. In other embodiments the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple esophageal samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a esophageal or a suspected esophageal tumor or neoplasm. In some cases, the fine needle aspirate sampling procedure may be guided by the use of an ultrasound, X-ray, or other imaging device.

In some embodiments of the present methods, a molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### IV. Additional Therapies

The current methods and compositions of the disclosure may include one or more additional therapies known in the art and/or described herein. In some embodiments, the additional therapy comprises an additional cancer treatment. Examples of such treatments are described herein.

In some embodiments, the additional therapy comprises an oncolytic virus. An oncolytic virus is a virus that preferentially infects and kills cancer cells. In some embodiments, the additional therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants. In some embodiments, the additional therapy comprises neoantigen administration. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden.

In some embodiments, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent. Suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

In some embodiments, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the additional therapy or prior therapy comprises surgery. Upon excision of part or all of cancerous cells, tissue, or tumor, a cavity may be formed in the body. Treatment may be accomplished by perfusion, direct injection, or local application of the area with an additional anti-cancer therapy. Such treatment may be repeated. Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

In some embodiments, the methods comprise or exclude administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Embodiments of the disclosure may include administration of ICB therapies, which are further described below.

In some embodiments, the immunotherapy comprises an inhibitor of a co-stimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

In some embodiments, the immunotherapy comprises cytokine therapy. Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins. Interferons are produced by the immune system. They are usually involved in anti-viral response, but also have use for cancer. They fall in three groups: type I (in particular IFNalpha and IFNbeta), type II and type III. Interleukins have an array of immune system effects. IL-2 is an exemplary interleukin cytokine therapy.

### A. Immunotherapy

Embodiments of the disclosure may include administration of ICB therapies, which are further described below.

### 1. PD-1, PD-L1, and PD-L2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PD-L1 on epithelial cells and tumor cells. PD-L2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PD-L1 activity.

Alternative names for “PD-1” include CD279 and SLEB2. Alternative names for “PD-L1” include B7-H1, B7-4, CD274, and B7-H. Alternative names for “PD-L2” include B7-DC, Btdc, and CD273. In some embodiments, PD-1, PD-L1, and PD-L2 are human PD-1, PD-L1 and PD-L2.

In some embodiments, the PD-1 inhibitor is a molecule that inhibits the binding of PD-1 to its ligand binding partners. In a specific aspect, the PD-1 ligand binding partners are PD-L1 and/or PD-L2. In another embodiment, a PD-L1 inhibitor is a molecule that inhibits the binding of PD-L1 to its binding partners. In a specific aspect, PD-L1 binding partners are PD-1 and/or B7-1. In another embodiment, the PD-L2 inhibitor is a molecule that inhibits the binding of PD-L2 to its binding partners. In a specific aspect, a PD-L2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PD-L2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PD-L1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PD-L2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PD-L1, or PD-L2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

### 2. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference. A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WOO 1/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein.

### V. CANCER THERAPY

In some embodiments, the method comprises or further comprises administering a cancer therapy to the patient. In some embodiments, the cancer therapy comprises an immunotherapy, such as adoptive T cell therapy or vaccination. Any of these cancer therapies may also be excluded. Combinations of these therapies may also be administered.

The term “cancer,” as used herein, may be used to describe a hematopoietic malignancy, a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some embodiments, the cancer is recurrent cancer. In some embodiments, the cancer is Stage I cancer. In some embodiments, the cancer is Stage II cancer. In some embodiments, the cancer is Stage III cancer. In some embodiments, the cancer is Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the methods and compositions may be for vaccinating an individual to prevent cancer. In certain embodiments the cells described herein can serve as a type of vaccine for ex vivo immunization and/or in vivo therapy in a mammal. In certain embodiments the mammal is a non-human mammal and in other embodiments the mammal is a human. With respect to ex vivo immunization, at least one of the following can occur in vitro prior to administering the cell into a mammal: i) expansion of the cells, ii) introducing a nucleic acid of the disclosure to the cells, and/or iii) cryopreservation of the cells.

The cells may be formulated in such a manner as to be suitable for delivery to a recipient without deleterious effects. They may or may not be formulated as a cell suspension. In specific cases they are formulated in a single dose form. They may be formulated for systemic or local administration. In some cases, the cells are formulated for storage prior to use, and the cell formulation may comprise one or more cryopreservation agents, such as DMSO (for example, in 10% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular embodiments the cells are in a formulated cell suspension that is stable at room temperature for 1, 2, 3, or 4 hours or more from time of thawing.

In some embodiments, the therapeutically effective or sufficient amount of the therapeutic composition or treatment administered to a human will be in the range of about 10² up to about 10¹⁰ cells per kg of patient body weight whether by one or more administrations. In some embodiments, the therapy used is about 10² cells to about 10⁹ cells/kg, about 10² cells to about 10⁸ cells/kg, about 10² cells to about 10⁷ cells/kg, about 10² cells to about 10⁶ cells/kg, about 10² cells to about 10⁵ cells/kg, about 10² cells to about 10⁴ cells/kg, or about 10² cells to about 10³ cells/kg administered daily, for example. In one embodiment, a therapy described herein is administered to a subject at a dose of about 10² cells, about 10³ cells, about 10⁴ cells, about 10⁵ cells, about 10⁶ cells, about 10⁷ cells, about 10⁸ cells, about 10⁹ cells, or about 10¹⁰ cells. The dose may be administered as a single dose or as multiple doses (e.g., 2 or 3 doses), such as infusions. The progress of this therapy is easily monitored by conventional techniques.

### VI. CELLS AND CELLULAR THERAPIES

### A. Cells

Certain embodiments relate to cells comprising polypeptides or nucleic acids of the disclosure. In some embodiments the cell is an immune cell or a T cell. “T cell” includes all types of immune cells expressing CD3 including T-helper cells, invariant natural killer T (iNKT) cells, cytotoxic T cells, T-regulatory cells (Treg) gamma-delta T cells, natural-killer (NK) cells, and neutrophils. The T cell may refer to a CD4+ or CD8+ T cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), human embryonic kidney (HEK) 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, Hut-78, Jurkat, HL-60, NK cell lines (e.g., NKL, NK92, and YTS), and the like.

In some instances, the cell is not an immortalized cell line, but is instead a cell (e.g., a primary cell) obtained from an individual. For example, in some cases, the cell is an immune cell obtained from an individual. As an example, the cell is a T lymphocyte obtained from an individual. As another example, the cell is a cytotoxic cell obtained from an individual. As another example, the cell is a stem cell (e.g., peripheral blood stem cell) or progenitor cell obtained from an individual.

### B. Cell Culture

In some embodiments, cells may be cultured for at least between about 10 days and about 40 days, for at least between about 15 days and about 35 days, for at least between about 15 days and 21 days, such as for at least about 15, 16, 17, 18, 19 or 21 days. In some embodiments, the cells of the disclosure may be cultured for no longer than 60 days, or no longer than 50 days, or no longer than 45 days. The cells may be cultured for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days or more than 40 days. The cells may be cultured in the presence of a liquid culture medium. Typically, the medium may comprise a basal medium formulation as known in the art. Many basal media formulations can be used to culture cells herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, and modifications and/or combinations thereof. Compositions of the above basal media are generally known in the art, and it is within the skill of one in the art to modify or modulate concentrations of media and/or media supplements as necessary for the cells cultured. In some embodiments, a culture medium formulation may be explants medium (CEM) which is composed of IMDM supplemented with 10% fetal bovine serum (FBS), 100 U/ml penicillin G, 100 μg/ml streptomycin and 2 mmol/L L-glutamine. Other embodiments may employ further basal media formulations, such as chosen from the ones above. In some aspects, the serum used in the growth of the cells may be human serum.

Any medium capable of supporting cells in vitro may be used to culture the cells. Media formulations that can support the growth of cells include, but are not limited to, Dulbecco's Modified Eagle's Medium (DMEM), alpha modified Minimal Essential Medium (αMEM), and Roswell Park Memorial Institute Media 1640 (RPMI Media 1640) and the like. Typically, up to 20% fetal bovine serum (FBS) or 1-20% horse serum is added to the above medium in order to support the growth of cells. A defined medium, however, also can be used if the growth factors, cytokines, and hormones necessary for culturing cells are provided at appropriate concentrations in the medium. Media useful in the methods of the disclosure may comprise one or more compounds of interest, including, but not limited to, antibiotics, mitogenic compounds, or differentiation compounds useful for the culturing of cells. The cells may be grown at temperatures between 27° C. to 40° C., such as 31° C. to 37° C., and may be in a humidified incubator. The carbon dioxide content may be maintained between 2% to 10% and the oxygen content may be maintained between 1% and 22%. The disclosure, however, should in no way be construed to be limited to any one method of isolating and culturing cells. Rather, any method of isolating and culturing cells should be construed to be included in the present disclosure.

For use in the cell culture, media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks' Balanced Salt Solution (HBSS), Earle's Salt Solution. Further antioxidant supplements may be added, e.g., β-mercaptoethanol. While many media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin. Also contemplated is supplementation of cell culture medium with mammalian plasma or sera. Plasma or sera often contain cellular factors and components that are necessary for viability and expansion. The use of suitable serum replacements is also contemplated.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed. In particular embodiments, cells are cultured in a cell culture system comprising a cell culture medium, preferably in a culture vessel, in particular a cell culture medium supplemented with a substance suitable and determined for protecting the cells from in vitro aging and/or inducing in an unspecific or specific reprogramming.

### C. Formulations and Culture of the Cells

In particular embodiments, the cells of the disclosure may be specifically formulated and/or they may be cultured in a particular medium. The cells may be formulated in such a manner as to be suitable for delivery to a recipient without deleterious effects.

The medium in certain aspects can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

In certain embodiments, the medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine). In specific embodiments, one or more of these may be explicitly excluded.

In some embodiments, the medium further comprises vitamins. In some embodiments, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some embodiments, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some embodiments, the vitamins include or consist essentially of biotin, DL alpha tocopherol acetate, DL alpha-tocopherol, vitamin A, or combinations or salts thereof. In some embodiments, the medium further comprises proteins. In some embodiments, the proteins comprise albumin or bovine serum albumin, a fraction of BSA, catalase, insulin, transferrin, superoxide dismutase, or combinations thereof. In some embodiments, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some embodiments, the medium comprises one or more of the following: a B-27® supplement, xeno-free B-27® supplement, GS21TM supplement, or combinations thereof. In some embodiments, the medium comprises or further comprises amino acids, monosaccharides, inorganic ions. In some embodiments, the amino acids comprise arginine, cystine, isoleucine, leucine, lysine, methionine, glutamine, phenylalanine, threonine, tryptophan, histidine, tyrosine, or valine, or combinations thereof. In some embodiments, the inorganic ions comprise sodium, potassium, calcium, magnesium, nitrogen, or phosphorus, or combinations or salts thereof. In some embodiments, the medium further comprises one or more of the following: molybdenum, vanadium, iron, zinc, selenium, copper, or manganese, or combinations thereof. In certain embodiments, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21TM supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. In specific embodiments, one or more of these may be explicitly excluded.

The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts. In specific embodiments, one or more of these may be explicitly excluded.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

In specific embodiments, the cells of the disclosure are specifically formulated. They may or may not be formulated as a cell suspension. In specific cases they are formulated in a single dose form. They may be formulated for systemic or local administration. In some cases, the cells are formulated for storage prior to use, and the cell formulation may comprise one or more cryopreservation agents, such as DMSO (for example, in 10% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular embodiments the cells are in a formulated cell suspension that is stable at room temperature for 1, 2, 3, or 4 hours or more from time of thawing.

In particular embodiments, the cells of the disclosure comprise an exogenous TCR, which may be of a defined antigen specificity. In some embodiments, the TCR can be selected based on absent or reduced alloreactivity to the intended recipient. In the example where the exogenous TCR is non-alloreactive, during T cell differentiation the exogenous TCR suppresses rearrangement and/or expression of endogenous TCR loci through a developmental process called allelic exclusion, resulting in T cells that express only the non-alloreactive exogenous TCR and are thus non-alloreactive. In some embodiments, the choice of exogenous TCR may not necessarily be defined based on lack of alloreactivity. In some embodiments, the endogenous TCR genes have been modified by genome editing so that they do not express a protein. Methods of gene editing such as methods using the CRISPR/Cas9 system are known in the art and described herein.

In some embodiments, the cells of the disclosure further comprise one or more chimeric antigen receptors (CARs). Examples of tumor cell antigens to which a CAR may be directed include at least 5T4, 8H9, αvβ6 integrin, BCMA, B7-H3, B7-H6, CAIX, CA9, CD19, CD20, CD22, CD30, CD33, CD38, CD44, CD44v6, CD44v7/8, CD70, CD123, CD138, CD171, CEA, CSPG4, EGFR, EGFR family including ErbB2 (HER2), EGFRvIII, EGP2, EGP40, ERBB3, ERBB4, ErbB3/4, EPCAM, EphA2, EpCAM, folate receptor-a, FAP, FBP, fetal AchR, FR□, GD2, G250/CAIX, GD3, Glypican-3 (GPC3), Her2, IL-13Ra2, Lambda, Lewis-Y, Kappa, KDR, MAGE, MCSP, Mesothelin, Mucd, Muc16, NCAM, NKG2D Ligands, NY-ESO-1, PRAME, PSC1, PSCA, PSMA, ROR1, SP17, Survivin, TAG72, TEMs, carcinoembryonic antigen, HMW-MAA, AFP, CA-125, ETA, Tyrosinase, MAGE, laminin receptor, HPV E6, E7, BING-4, Calcium-activated chloride channel 2, Cyclin-B1, 9D7, EphA3, Telomerase, SAP-1, BAGE family, CAGE family, GAGE family, MAGE family, SAGE family, XAGE family, NY-ESO-1/LAGE-1, PAME, SSX-2, Melan-A/MART-1, GP100/pmel17, TRP-1/-2, P. polypeptide, MC1R, Prostate-specific antigen, β-catenin, BRCA1/2, CML66, Fibronectin, MART-2, TGF-βRII, or VEGF receptors (e.g., VEGFR2), for example. The CAR may also be specific for the Tn-glycosylation that can occur in surface proteins due to mutations in the COSMC gene (see, for example, He et al. 2019, JCI Insight. 2019; 4(21):e130416 and Posey AD Jr et al., Immunity. 2016 Jun. 21; 44(6):1444-54, which are herein incorporated by reference). The CAR may be a first, second, third, or more generation CAR or affinity matured variants thereof (see, for example, Sharma et al, PNAS Jun. 30, 2020 117 (26) 15148-15159, which is herein incorporated by reference). The CAR may be bispecific for any two nonidentical antigens, or it may be specific for more than two non-identical antigens. In some aspects, the CAR comprises the 237 CAR described in WO2020056023 and Sharma et al, PNAS Jun. 30, 2020 117 (26) 15148-15159, each of which are herein incorporated by reference. In some aspects, the CAR comprises the 5E5 CAR described in Posey AD Jr et al., Immunity. 2016 Jun. 21; 44(6):1444-54, which is herein incorporated by reference.

### D. Cell Generation

Certain methods of the disclosure concern culturing the cells obtained from human tissue samples. In particular embodiments of the present disclosure, cells are plated onto a substrate that allows for adherence of cells thereto. This may be carried out, for example, by plating the cells in a culture plate that displays one or more substrate surfaces compatible with cell adhesion. When the one or more substrate surfaces contact the suspension of cells (e.g., suspension in a medium) introduced into the culture system, cell adhesion between the cells and the substrate surfaces may ensue. Accordingly, in certain embodiments cells are introduced into a culture system that features at least one substrate surface that is generally compatible with adherence of cells thereto, such that the plated cells can contact the said substrate surface, such embodiments encompass plating onto a substrate, which allows adherence of cells thereto.

Cells of the disclosure may also be grown free floating in culture medium (suspension culture) without being attached to a surface. In some aspects, the cells can be grown to a higher density in suspension culture compared to adherent cells.

Cells of the present disclosure may be identified and characterized by their expression of specific marker proteins, such as cell-surface markers. Detection and isolation of these cells can be achieved, for example, through flow cytometry, ELISA, and/or magnetic beads. Reverse-transcription polymerase chain reaction (RT-PCR) may be used to quantify cell-specific genes and/or to monitor changes in gene expression in response to differentiation. In certain embodiments, the marker proteins used to identify and characterize the cells are selected from the list consisting of c-Kit, Nanog, Sox2, Hey1, SMA, Vimentin, Cyclin D2, Snail, E-cadherin, Nkx2.5, GATA4, CD105, CD90, CD29, CD73, Wt1, CD34, CD45, and a combination thereof.

### E. Adoptive Cell Therapy

Adoptive Cell Therapy is a form of passive immunization by the transfusion (adoptive cell transfer) of immune cells, in particular T-cells. T cells are found in blood and tissue and usually activate when they find foreign pathogens or other antigens that T-cell's surface receptors encounter parts of foreign proteins (antigens) that are displayed on surface of other cells. These latter cells can be either infected cells, or antigen presenting cells (APCs) that are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the expansion and the reinfusion of the resulting cells. Activation can take place through gene therapy, or by exposing the T cells to tumor antigens. Additional details on the preparation, selection, use, combination with other therapies, an/or administration of cells for ACT treatment are described in the literature (Cook K et al., 2018, Elahi R et al., 2018; Sharma P. et al., 2017).

In some embodiments, the adoptive cell therapy comprises dendritic cell therapy, which provokes anti-tumor responses by causing dendritic cells to present tumor antigens to lymphocytes, and then activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T. One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF. Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies may include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

In some embodiments, the adoptive cell therapy comprises CAR-T cell therapy. Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy. Exemplary CAR-T therapies include Tisagenlecleucel (Kymriah) and Axicabtagene ciloleucel. In some embodiments, the CAR-T therapy targets CD19 or CD20.

### VII. ENGINEERED T CELL RECEPTORS

T-cell receptors comprise two different polypeptide chains, termed the T-cell receptor α (TCRα) and β (TCRβ) chains, linked by a disulfide bond. These α:β heterodimers are very similar in structure to the Fab fragment of an immunoglobulin molecule, and they account for antigen recognition by most T cells. A minority of T cells bear an alternative, but structurally similar, receptor made up of a different pair of polypeptide chains designated γ and δ. Both types of T cell receptor differ from the membrane-bound immunoglobulin that serves as the B-cell receptor: a T cell receptor has only one antigen-binding site, whereas a B-cell receptor has two, and T-cell receptors are never secreted, whereas immunoglobulin can be secreted as antibody.

Both chains of the T-cell receptor have an amino-terminal variable (V) region with homology to an immunoglobulin V domain, a constant (C) region with homology to an immunoglobulin C domain, and a short hinge region containing a cysteine residue that forms the interchain disulfide bond. Each chain spans the lipid bilayer by a hydrophobic transmembrane domain, and ends in a short cytoplasmic tail.

The three-dimensional structure of the T-cell receptor has been determined. The structure is indeed similar to that of an antibody Fab fragment, as was suspected from earlier studies on the genes that encoded it. The T-cell receptor chains fold in much the same way as those of a Fab fragment, although the final structure appears a little shorter and wider. There are, however, some distinct differences between T-cell receptors and Fab fragments. The most striking difference is in the Cα domain, where the fold is unlike that of any other immunoglobulin-like domain. The half of the domain that is juxtaposed with the Cβ domain forms a β sheet similar to that found in other immunoglobulin-like domains, but the other half of the domain is formed of loosely packed strands and a short segment of a helix. The intramolecular disulfide bond, which in immunoglobulin-like domains normally joins two p strands, in a Cα domain joins a β strand to this segment of a helix.

There are also differences in the way in which the domains interact. The interface between the V and C domains of both T-cell receptor chains is more extensive than in antibodies, which may make the hinge joint between the domains less flexible. And the interaction between the Cα and Cβ domains is distinctive in being assisted by carbohydrate, with a sugar group from the Cα domain making a number of hydrogen bonds to the Cβ domain. Finally, a comparison of the variable binding sites shows that, although the complementarity-determining region (CDR) loops align fairly closely with those of antibody molecules, there is some displacement relative to those of the antibody molecule. This displacement is particularly marked in the Vα CDR2 loop, which is oriented at roughly right angles to the equivalent loop in antibody V domains, as a result of a shift in the β strand that anchors one end of the loop from one face of the domain to the other. A strand displacement also causes a change in the orientation of the Vβ CDR2 loop in two of the seven Vβ domains whose structures are known. As yet, the crystallographic structures of seven T cell receptors have been solved to this level of resolution.

Embodiments of the disclosure relate to engineered T cell receptors. The term “engineered” refers to T cell receptors that have TCR variable regions grafted onto TCR constant regions to make a chimeric polypeptide that binds to peptides and antigens of the disclosure. In certain embodiments, the TCR comprises intervening sequences that are used for cloning, enhanced expression, detection, or for therapeutic control of the construct, but are not present in endogenous TCRs, such as multiple cloning sites, linker, hinge sequences, modified hinge sequences, modified transmembrane sequences, a detection polypeptide or molecule, or therapeutic controls that may allow for selection or screening of cells comprising the TCR.

In some embodiments, the exogenous TCR comprises proteins expressed from TCR-alpha and TCR-beta genes. In some embodiments, the exogenous TCR comprises proteins expressed from TCR-gamma and TCR-delta genes. In some embodiments, the exogenous TCR comprises proteins expressed from TCR-alpha and TCR-beta genes and the antigen recognition receptor comprises proteins expressed from the TCR-gamma and TCR-delta genes. In some embodiments, the exogenous TCR comprises proteins expressed from TCR-gamma and TCR-delta genes and the antigen recognition receptor comprises proteins expressed from the TCR-alpha and TCR-beta genes.

Methods of generating antigen-specific TCRs are known in the art. Methods may include, for example, 1) Synthesizing known or predicted HLA-restricted peptide epitopes derived from proteins of interest (e.g. tumor antigens, neoantigens from sequencing data, etc.); 2) presenting these via an antigen-presenting cell (for expansion) or tetramer (for direct sorting) to a pool of T cells from which TCR sequences are to be extracted (e.g. tumor infiltrating lymphocytes in the case of tumor-ag specific T cells); 3) selecting or screening for antigen-specific T cells (e.g. FACS sorting antigen-specific T cells based on tetramer binding); 4) cloning (via RT-PCR) and sequencing the TCR genes (i.e. alpha and beta chains or gamma and delta chains of the TCRs); cloning and sequencing may be done either on a population or single cell level; and 5) confirming and analyzing TCR specificity by, for example, testing the function of TCR clones by engineering peripheral blood T cells with these sequences and assessing their reactivity to target cells that express the cognate peptide-MHC complex. Reactivity is usually measured based on cytokine production (e.g. interferon gamma).

In some embodiments, the TCR comprises non-TCR sequences. Accordingly, certain embodiments relate to TCRs with sequences that are not from a TCR gene. In some embodiments, the TCR is chimeric, in that it contains sequences normally found in a TCR gene, but contains sequences from at least two TCR genes that are not necessarily found together in nature.

### VIII. PHARMACEUTICAL COMPOSITIONS

The present disclosure includes methods for treating disease and modulating immune responses in a subject in need thereof. The disclosure includes cells that may be in the form of a pharmaceutical composition that can be used to induce or modify an immune response.

Administration of the compositions according to the current disclosure will typically be via any common route. This includes, but is not limited to parenteral, orthotopic, intradermal, subcutaneous, orally, transdermally, intramuscular, intraperitoneal, intraperitoneally, intraorbitally, by implantation, by inhalation, intraventricularly, intranasally or intravenous injection.

Typically, compositions and therapies of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most about or at least about 3, 4, 5, 6, 7, 8, 9, 10 or more. The administrations may range from 2-day to 12-week intervals, more usually from one to two week intervals. The course of the administrations may be followed by assays for alloreactive immune responses and T cell activity.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

The compositions of the disclosure can be formulated for parenteral administration, e.g., formulated for injection via the intravenous, intramuscular, sub-cutaneous, or even intraperitoneal routes. Typically, such compositions can be prepared as injectables, either as liquid solutions or suspensions and the preparations can also be emulsified.

The pharmaceutical forms suitable for injectable use include sterile aqueous solutions or dispersions; formulations including sesame oil, peanut oil, or aqueous propylene glycol. It also should be stable under the conditions of manufacture and storage and must be preserved against the contaminating action of microorganisms, such as bacteria and fungi.

Sterile injectable solutions are prepared by incorporating the active ingredients (i.e. cells of the disclosure) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

An effective amount of a composition is determined based on the intended goal. The term “unit dose” or “dosage” refers to physically discrete units suitable for use in a subject, each unit containing a predetermined quantity of the composition calculated to produce the desired responses discussed herein in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

The compositions and related methods of the present disclosure, particularly administration of a composition of the disclosure may also be used in combination with the administration of additional therapies such as the additional therapeutics described herein or in combination with other traditional therapeutics known in the art.

The therapeutic compositions and treatments disclosed herein may precede, be co-current with and/or follow another treatment or agent by intervals ranging from minutes to weeks. In embodiments where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administerable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some embodiments, the therapeutically effective or sufficient amount of the immune checkpoint inhibitor, such as an antibody and/or microbial modulator, that is administered to a human will be in the range of about 0.01 to about 50 mg/kg of patient body weight whether by one or more administrations. In some embodiments, the therapy used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In one embodiment, a therapy described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### IX. KITS

Certain aspects of the present disclosure also concern kits containing nucleic acids, vectors, or cells of the disclosure. The kits may be used to implement the methods of the disclosure. In some embodiments, kits can be used to evaluate or facilitate neoantigen library construction or transfection. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more nucleic acid probes, primers, or synthetic RNA molecules, or any value or range and combination derivable therein. In certain embodiments, the kits may comprise materials for analyzing cell morphology and/or phenotype, such as histology slides and reagents, histological stains, alcohol, buffers, tissue embedding mediums, paraffin, formaldehyde, and tissue dehydrant.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, polypeptide or polynucleotide detecting agents of the disclosure for drug discovery are contemplated.

In certain aspects, negative and/or positive control agents are included in some kit embodiments. The control molecules can be used to verify transfection efficiency and/or control for transfection-induced changes in cells.

Embodiments of the disclosure include kits for analysis of a pathological sample by assessing a nucleic acid or polypeptide profile for a sample comprising, in suitable container means, two or more RNA probes or primers for detecting expressed polynucleotides. Furthermore, the probes or primers may be labeled. Labels are known in the art and also described herein. In some embodiments, the kit can further comprise reagents for labeling probes, nucleic acids, and/or detecting agents. The kit may also include labeling reagents, including at least one of amine-modified nucleotide, poly(A) polymerase, and poly(A) polymerase buffer. Labeling reagents can include an amine-reactive dye. Kits can comprise any one or more of the following materials: enzymes, reaction tubes, buffers, detergent, primers, probes, antibodies. In some embodiments, these kits include the needed apparatus for performing RNA extraction, RT-PCR, and gel electrophoresis. Instructions for performing the assays can also be included in the kits.

The kits may further comprise instructions for using the kit for assessing expression, means for converting the expression data into expression values and/or means for analyzing the expression values to generate ligand/receptor interaction data.

Kits may comprise a container with a label. Suitable containers include, for example, bottles, vials, and test tubes. The containers may be formed from a variety of materials such as glass or plastic. The container may hold a composition which includes a probe that is useful for the methods of the disclosure. The kit may comprise the container described above and one or more other containers comprising materials desirable from a commercial and user standpoint, including buffers, diluents, filters, needles, syringes, and package inserts with instructions for use.

### X. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Neo-T—Personalized Medicine: T Cells Engineered with Neoantigen-Specific T Cell Receptors for Adoptive T Cell Therapy

The selection of appropriate target structures, so-called antigens, on cancers is essential for cellular therapies using the patient's own T cells engineered with tumor-specific receptors. However, the tremendous diversity of cancer makes it impossible to identify tumor antigens that serve as targets in different tumor entities and thus larger groups of patients. The approach described herein breaks away from the short-sighted strategy of making T cell therapy a treatment option that depends on pre-defined tumor antigens, as each individual cancer comes with a set of the most desirable target structures—neoantigens. Neoantigens are a common characteristic of all cancers, as they are the result of somatic mutations, most of which were causative for tumor development. Using system medicine, determining neoantigens is a rapid and cost-efficient procedure based on comparing the genetic material of a tumor biopsy with patient reference tissue. Based on the inventors' decade-long research, they believe that T-cell therapy targeting neoantigens has the potential not only to delay tumor growth, but to eradicate cancer in its entirety. This is achieved by selecting a suitable pair of T cell receptors (TCRs) that recognize neoantigens when presented in tumor tissue by patient MHC molecules. This approach requires that one of the two TCRs must recognize a neoantigen in the context of MCH class I and the other in the context of MHC class II. This approach achieves highest possible safety as the patient's own T cells are used to identify therapeutic TCRs targeting neoantigens expressed uniquely in the patient's own tumor. This eliminates the risk of unpredictable cross-reactivity, which is frequently ignored or subsequently encountered when using TCRs from other sources. To increase the number of neoantigen-specific T cells in a patient prior to TCR isolation, a vaccination strategy was developed that uses non-malignant surrogate cancer cells (SCCs) that originate from the patient's own healthy B cells. SCCs will also be the central tool for the unbiased selection of optimal target structures from the entire pool of patient neoantigens and MHCs. The individual steps, detailed below, are incorporated into an approach termed “Neo-T”. Neo-T allows the use of neoantigens of any given tumor as target structures for adoptively transferred T cells and provides a novel therapy design that is distinct from existing approaches. The unique elements of Neo-T open the door to a universal, individualized T-cell therapy that will facilitate a paradigm shift for the design of safe and efficient immunotherapies that are personalized in its literal sense.

Neo-T can be divided into the following steps, based on the proprietary elements that were designed.

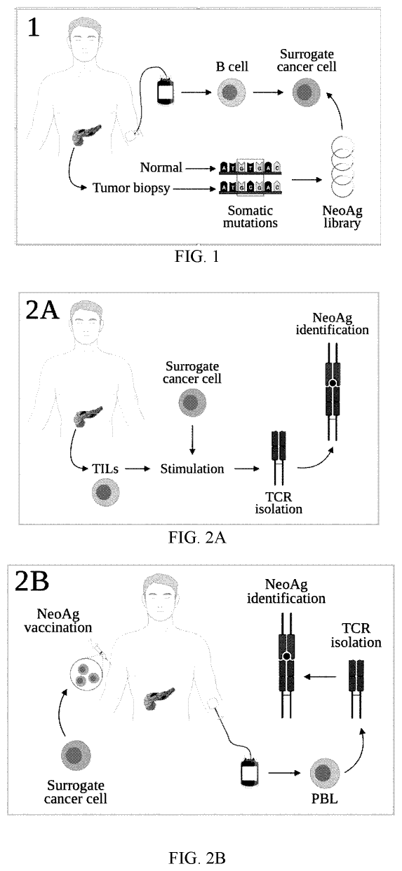

STEP 1 (FIG. 1): Identification of somatic mutations and generation of surrogate cancer cells (SCCs). The basis of the Neo-T approach is sequencing of the genetic material (DNA, RNA) from tumor tissue and normal reference material obtained from the patient. This usually requires only a small amount of tissue obtained by minimally invasive biopsy or blood collection. From the resulting list of somatic mutations neoantigen-encoding gene cassettes will be designed, which represent the patient's neoantigen library. For the production of SCCs, patient B cells are converted into long-lived but non-malignant cell lines and neoantigen expression is inserted by transferring the respective gene library. For the subsequent induction of neoantigen-specific T cells (STEP 2), SCCs are the crucial tool and offer two different strategies (A and B).

STEP 2A (FIG. 2A): Isolation of neoantigen-specific T cells from patient tumor-infiltrating lymphocytes. Neoantigen-specific T cells can be isolated directly from the tumor tissue. Since the broad diversity of lymphocytes that migrate into the tumor would preclude a direct isolation of neoantigen-specific T cells, SCCs can be used to enrich the desired T cell populations in vitro. In contrast to the inhibitory effects that would arise from stimulation with tumor cells, the use of SCCs can overcome the suppressive environment even allowing the expansion of terminally exhausted TILs. Furthermore, because SCCs contain the entire pool of neoantigens and MHCs of each individual patient, this strategy allows for an unbiased search for optimal target structures. The neoantigen library is used to determine the specificity of each identified TCR, and the inventors have developed special protocols to determine the quality of neoantigens as targets. SCCs allow for the selective activation and expansion of neoantigen-specific T cells derived from a population of tumor infiltrating lymphocytes (TILs). CD4⁺ TILs are stimulated by MHC II-presented neoantigens when loaded on autologous CD40L-induced B cells (or dendritic cells) as lysates generated either from SCCs or directly from tumor material.

STEP 2B (FIG. 2B): Isolation of neoantigen-specific T cells from patient peripheral blood. In the majority of cases, the frequency of neoantigen-specific T cells in the peripheral blood of a patient is too low to be a source for the isolation of specific TCRs. Using SCCs, patients can be immunized against their tumor neoantigens. While this vaccination may also achieve therapeutic effects, the protocol is designed to enrich neoantigen-specific T cells in the peripheral blood of the patient so that their TCRs can be identified and the neoantigens they recognize. Thus, SCCs can be used for stimulation of neoantigen-specific CD8⁺ as well as CD4⁺ T cell by vaccination. Furthermore, SCCs can be used for neoantigen identification and characterization, and the protocols for identification and characterization of neoantigen targets, particularly for using lysates to determine the quality of MHC II-presented neoantigens represent novel and inventive aspects of the disclosure.

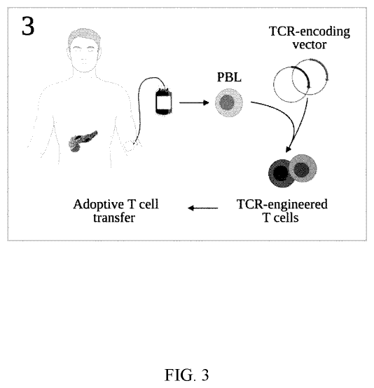

STEP 3 (FIG. 3): Adoptive therapy using T cells engineered with neoantigen-specific TCRs. The Neo-T protocol for isolating neoantigen-specific T cells from tumor infiltrating T cells or peripheral blood is sufficiently versatile to identify TCRs for combination T cell therapy. The inventors have developed a therapy design that facilitates tumor eradication using only two neoantigen-specific TCRs. One TCR is introduced into CD8⁺ T cells and targets cancer cells directly via recognition of neoantigens presented on MHC-I. The second TCR is transferred into CD4⁺ T cells and recognizes neoantigens presented via MHC-II on cells of the tumor stroma. This T cell therapy approach requires the production of two cell products for each patient. The Neo-T approach of using the transfer of CD4⁺ T cells to stabilize tumor growth and subsequently eradicate the tumor in combination with CD8⁺ T cells provides for a novel cancer therapy with the potential to eradicate tumors.

These aspects are further described below.

### A. Generation of Surrogate Cells

(I) B cells from each patient will be stimulated with an irradiated CD40L-positive cell line and IL-4 to induce continuous proliferation of the B cells that express all autologous MHC alleles (up to 6 MHC I and 6 MHC II) of the patient. This procedure conditionally immortalizes the autologous B cells (CIBs). CIBs will be used as: (a) autologous normal specificity control when selecting therapeutic TCRs, and (b) as source of autologous antigen-presenting cells (APCs).

(II) In parallel, tumor tissue from the same patient will be analyzed to determine non-synonymous single nucleotide variants (nsSNVs) and frame-shift somatic mutations by whole-exome and RNA sequencing. Based on these nsSNVs, tandem “minigene” vectors will be constructed. Each vector will consist of ten different minigenes, each encoding for 25 amino acids containing the mutated or wildtype codon at the center position. Each minigene will be separated from the next by a proteasomal cleavage site (amino acid sequence: AAY). The tandem array of the 10 minigenes will be linked by a 2A-element with CD11c as B-cell-independent, non-immunogenic marker. The vector construct containing the mutant minigenes is referred to as TMM (Tandem Mutant Minigenes), while the construct containing the wildtype minigenes is referred to as TNM (Tandem Normal Minigenes). Usually multiple different TMMs and TNMs need to be constructed for each patient (e.g. TMM¹⁻¹⁰, TMM¹¹⁻²⁰, TMM²¹⁻³⁰, TMM³¹⁻⁴⁰, TMM⁴¹⁻⁵⁰ etc.) to cover the entire number of nsSNVs and their normal counterpart in the patient cancer (the average number of nsSNVs for ovarian cancer is 42 and for pancreatic cancer).

(IIIa) TMMs will be introduced (by electroporation or viral transduction) into the CIBs, generating a cell that expresses the mutant neoepitopes. The combination of CD19⁺ and CD11c will be used to sort and enrich TMM-expressing CIBs. Unlike approaches where mutant neoepitopes are artificially loaded onto target cells, our approach includes natural DNA translation and MHC processing of mutant neoepitopes by the cancer patient's specific presentation machinery and predicts which mutant neoepitopes are better presented by patient's cancer cells. The inventors name these CIBs that express the individual, patient-specific mutant neoantigens “surrogate cancer cells”, or in short “SCC”. At the average, each patient's cancer will be represented by a pool of five SCCs, e.g. SCC¹⁻¹⁰ SCC¹¹⁻²⁰, SCC²¹⁻³⁰, SCC³¹⁻⁴⁰, SCC⁴¹⁻⁵⁰ each expressing TMM¹⁻¹⁰, TMM¹⁻²⁰, TNM²¹⁻³⁰, TNM³¹⁻⁴⁰, TNM⁴¹⁻⁵⁰ respectively.

(IIIb) TNMs will be introduced (by electroporation or viral transduction) into the CIBs, generating a cell that expresses normal self-epitopes from the peptidomal repertoire of pancreatic- or ovarian-derived cells which are usually not present in normal B cells. This approach also includes natural DNA translation and MHC processing of self-epitopes. Afterwards, the combination of CD19⁺ and CD11c will be used to sort and enrich TNM-expressing CIBs. The inventors name these CIBs that express the individual, patient-specific normal self-antigens “surrogate normal cells”, or in short “SNC”. The SNCs will be used as safety control to exclude TCRs that are able to recognize both, mutant and self-epitopes. At the average, each patient's normal tissue will be represented by a pool of five SNCs, e.g. SNC¹⁻¹⁰, SNC¹¹⁻²⁰, SNC²¹⁻³⁰, SNC³¹⁻⁴⁰, SNC⁴¹⁻⁵⁰ each expressing TNM¹⁻¹⁰, TNM¹¹⁻²⁰, TNM²¹⁻³⁰, TNM³¹⁻⁴⁰, TNM⁴¹⁻⁵⁰ respectively.

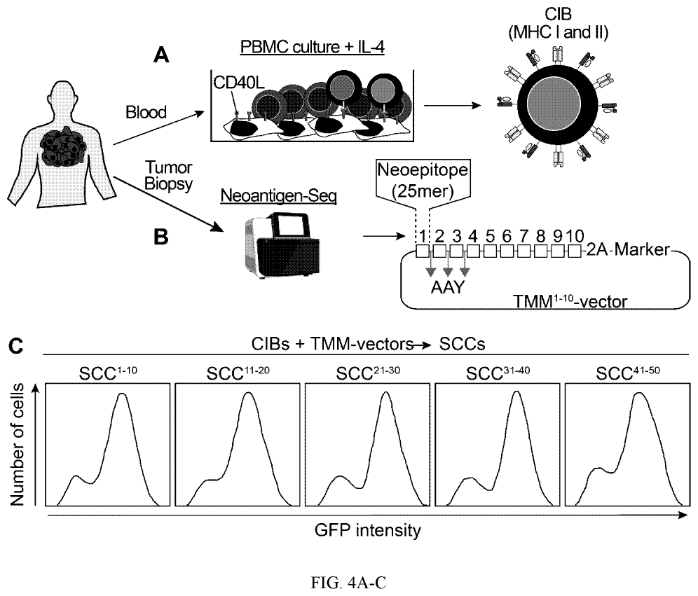

FIG. 4 outlines the generation of the surrogate cancer cell (SCC). From a cancer patient, blood will be drawn (˜20 mL) and peripheral blood mononuclear cells (PBMC) will be cultured with irradiated, CD40L-expressing cells together with IL-4 to stimulate B cell proliferation (FIG. 4A). This way autologous CIBs, which express all MHCs of the patient, are established. In parallel, the patient's neoantigens are identified by sequencing tumor material from a biopsy. The neoantigen sequences will be used to generate the TMMs (FIG. 4B). In the end, the TMMs will be introduced into the CIBs which become positive for the marker encoded on the TMM and indicate that a SCC with proper expression level has been generated. In the example of the ovarian cancer patient shown in FIG. 4C, 50 neoantigens were identified by sequencing of a tumor biopsy. Five TMMs (TMM¹⁻¹⁰, TMM¹¹⁻²⁰, TMM²¹⁻³⁰, TMM³¹⁻⁴⁰ and TMM⁴¹⁻⁵⁰) were constructed, each encoding 10 different neoantigens. Each of the five TMMs was introduced into separate CIBs generating five different and marker-positive SCCs (SCC¹⁻¹⁰, SCC¹¹⁻²⁰, SCC²¹⁻³⁰ SCC³¹⁻⁴⁰ and SCC⁴¹⁻⁵⁰).

### B. Expansion of Mutant Neoantigen-Specific CD8⁺ T Cells by SCC's

The SCCs will be used for in vitro stimulation of TILs and peripheral blood T cells to identify mutant neoantigen-specific T cells. Cross-reactive T cells will be detected and disregarded by using SNCs (Innovation 1, Part (IIIb)) as autologous control.

### C. Expansion of Mutant Neoantigen-Specific CD4⁺ T Cells with Lysates Generated from SCC's

The SCCs will be lysed by three cycles of freezing (liquid nitrogen) and thawing (37° C. water bath). The generated SCC-lysates will be cultured with CIBs or PBL-derived DCs and either TILs or peripheral blood T cells to identify mutant neoantigen-specific T cells. As an autologous control, cell lysate of SNCs will be used. With the use of lysates cultured together with APCs, the MHC class II antigen presentation pathway is employed and natural antigen presentation together with MHC class II loading in the background of the patient's APC machinery is conducted.

### D. Expansion of Mutant Neoantigen-Specific CD4⁺ T Cells with Lysates from Tumor Tissue

If sufficient tumor tissue is available, the tumor tissue will be lysed by three cycles of freezing (liquid nitrogen) and thawing (37° C. water bath) and cultured with CIBs or PBL-derived DC's together with either TILs or peripheral blood T cells to identify mutant neoantigen-specific T cells. As an autologous control, cell lysate of SNCs will be used. In addition to the use of the natural MHC class II processing pathway as described under Innovation 3A, using lysate of tumor tissue has the benefit of testing the natural, unmanipulated amounts of antigen as they are available in the tumor tissue for T cell stimulation. Such lysates are usually in sparse supply due to limited availability of tumor tissue but when available, such lysates will be used as a final “reality check” to confirm TCRs selected with lysates of SCCs. Tumor tissue lysates are a more potent source of tumor-specific antigen because mutant neoantigen-specific CD4⁺ T cells would be stimulated and expand in response to natural amounts of MHC II-presented neoantigen.

Use of the SCCs as viable cell for stimulation of mutant neoantigen-specific, autologous CD8⁺ T cells is a unique aspect of the disclosure. In addition, using lysates of SCCs or lysates from SNCs or additional tumor tissue to stimulate mutant neoantigen-specific, autologous CD4⁺ T cells is a further unique aspect of the disclosure.

### E. Using the SCCs for Mutant Neoantigen-Specific Vaccination

Antigen processing and presentation needs to be consistent over a long period of time for successful mutant neoantigen-specific vaccination. Therefore, the TMMs will be stably integrated (viral transduction or transposase or CRISPR/Cas9s) into autologous B cells and a GMP-grade SCC line will be generated. The combination of CD19⁺ and TMM-linked marker expression on the SCCs can be used to sort and enrich mutant neoantigen expressing cells and track the cells in vivo. Due to the use of an autologous, non-immunogenic marker, the SCCs only induce an immune response to the introduced mutant neoantigens encoded on the TMM constructs. In some instances, SCCs will be made proliferation-incompetent by irradiation in vitro before use for vaccination in vivo. Using autologous SCCs for vaccination to induce mutant neoantigen-specific CD4⁺ as well as CD8⁺ T cells is a unique approach.

### F. Identification and Verification of Mutant Neoantigen-Specific, Autologous TCR-Engineered T Cells Using SCCs

Once mutant neoantigen-specific T cells have been identified, their TCR genes will be isolated and used to engineer (either by viral transduction or CRISPR/Cas) autologous T cells to express these TCRs. Their reactivity will be confirmed with viable SCCs for TCR-engineered autologous CD8⁺ T cells and with lysates of SCCs or tumor lysates (if available) cultured with autologous CIBs for TCR-engineered autologous CD4⁺ T cells. As an autologous control, SNCs either as viable cells or as cell lysates will be used. Final specificity control and exclusion of self-reactive TCRs will be performed by using 25mer mutant and wildtype peptides. Thus, a cancer cell recognizing CD8⁺ TCR (CARE-TCR) and a lysate recognizing CD4⁺ TCR (LYRE-TCR) will be identified and used for effective adoptive T cell transfer.

The SCCs and SNCs, either as viable cells or in form of lysates will be used to verify specificity and therapeutic efficiency of TCR-engineered autologous CD8⁺ and CD4⁺ T cells. Therefore, the use of SCCs and SNCs for TCR-verification are also unique aspects of the disclosure.

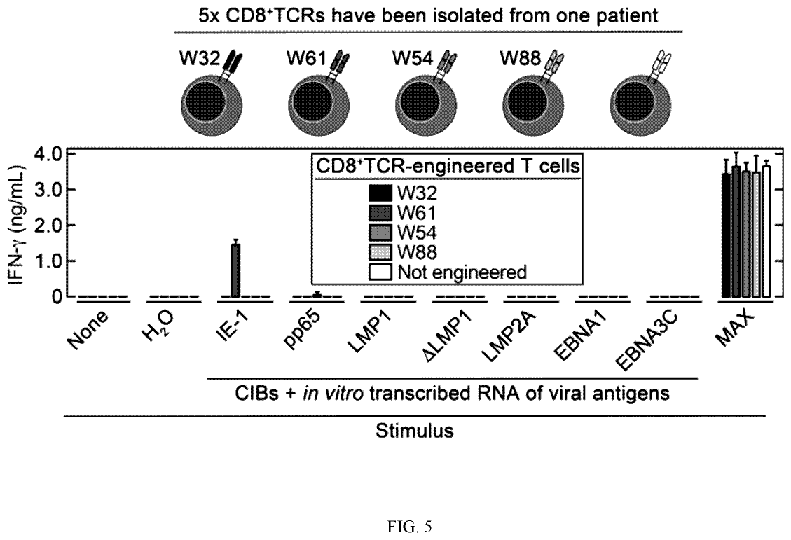

For identification of CARE-TCR-engineered CD8⁺ T cells, they will be cultured with SCCs and reactivity will be determined by IFN-γ secretion (FIG. 5). Five different CD8⁺ TCRs were identified from one patient. These CD8⁺ TCRs were used to TCR-engineer CD8⁺ T cells which were then co-cultured with CIBs that were electroporated with in vitro transcribed RNA of viral antigens. This way, one CD8*TCR was identified to be specific for the CMV-derived antigen IE-1. This assay shows proof of principle that CIBs which express antigens (viral or tumor neoantigens) can function as SCCs and be used to stimulate and identify specific CARE-TCRs.

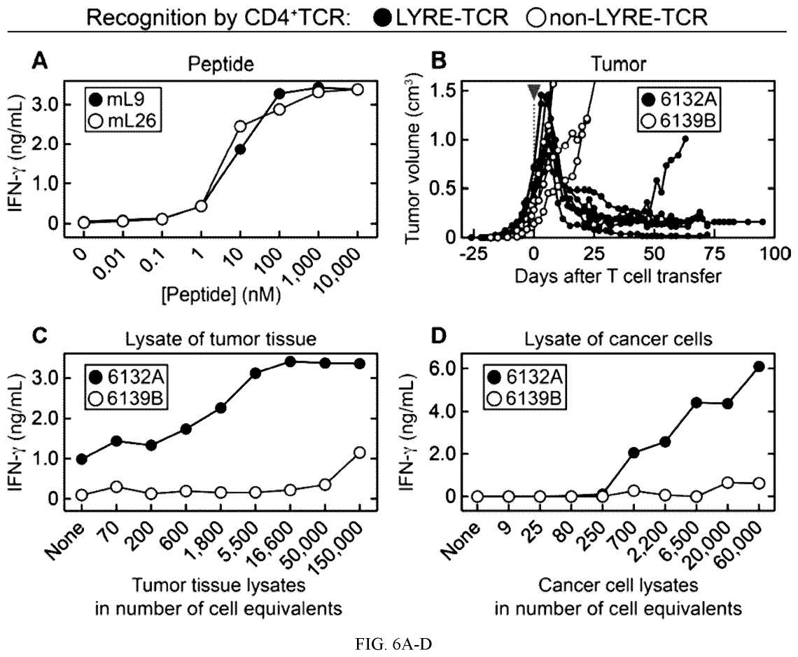

The therapeutic efficiency of TCR-engineered CD4⁺ T cells depends on their ability to recognize mutant neoantigens that are presented on APCs after being processed from lysates of either tumor tissue or cancer cells (FIG. 6). Two mutant neoantigen-specific CD4⁺ TCRs can recognize mutant neoantigen peptides presented on APCs similarly determined by IFN-γ secretion (FIG. 6A). However, only one CD4⁺ TCR mediates tumor destruction followed by growth arrest and stable disease when TCR-engineered CD4⁺ T cells are used for adoptive T cell transfer against large and long-established tumors (FIG. 6B). The different outcomes in vivo can be predicted in vitro when lysates generated from either tumor tissue (FIG. 6C) or cancer cells (FIG. 6D) are being used to stimulate CD4⁺ TCR-engineered T cells. One CD4⁺ TCR is able to recognize lysates that are processed and presented by APCs, while the other CD4⁺ TCR fails to show the same reactivity. Thus, a LYRE-TCR will be used and is required for effective adoptive T cell transfer.

### G. Individualized, Cellular Therapy Strategy that Uses a Combination of Autologous, Mutant Neoantigen-Specific CD8⁺ and CD4⁺ TCR-Engineered T Cells

This approach of individualized cellular therapy aims to identify at least two mutant neoantigen-specific TCRs from the cancer patients own repertoire either from TIL cultures or after vaccination from peripheral blood T cells. It is proposed that one TCR needs to recognize mutant MHC class I restricted neoantigen on the cancer cell surface named CARE-TCR, while the second TCR must recognize an MHC class II restricted mutant neoantigen from cancer cell lysates, LYRE-TCR. The inventors' proprietary preclinical data indicate that combination of a LYRE-TCR with a CARE-TCR is necessary and sufficient for the eradication of tumors. Therapy will be conducted by infusion of two different TCR-engineered autologous T-cell products each containing one TCR: one TCR targeting an MHC class I restricted mutant neoantigen and the other TCR targeting an MHC class II restricted neoantigen. The approach is designed so that both types of TCRs can be identified and be given at the same time. The inventors' proprietary preclinical data also indicate that a LYRE-TCR (targeting an MHC class II restricted mutant neoantigen) has priority, as it can extend the patient's life and gain additional time that might be needed to find the other CARE-TCR targeting an MHC class I restricted mutant neoantigen if a CARE-TCR is not immediately available.

The strategy of requiring both a TCR targeting an MHC class I restricted mutant neoantigen and a TCR targeting an MHC class II restricted neoantigen for individualized adoptive cellular therapy represents a novel aspect of the disclosure.

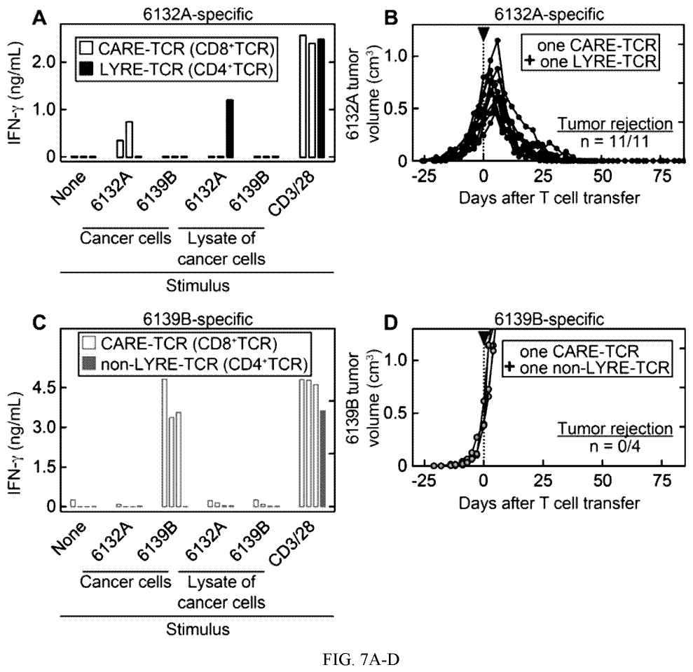

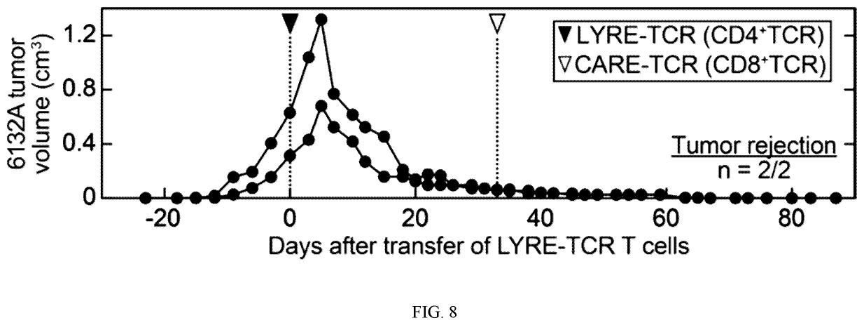

One CARE-TCR and one LYRE-TCR are both essential and sufficient for effective tumor eradication by adoptive T cell transfer (FIG. 7). Two CARE-TCRs and one LYRE-TCR were isolated in the 6132A tumor model (FIG. 7A). One CARE-TCR expressing CD8⁺ T cell population together with one LYRE-TCR expressing CD4⁺ T cell population was able to achieve tumor eradication when used in combination for adoptive T cell transfer against large established 6132A tumors (FIG. 7B). In a second tumor model (6139B) three CARE-TCRs were isolated together with one non-LYRE-TCR (FIG. 7C). The combination of one CARE-TCR with one non-LYRE-TCR used for adoptive T cell transfer against large established tumors failed to achieve tumor eradication (FIG. 7D). Even though the CARE-TCRs seems to perform better in vitro against 6139B than the CARE-TCRs used against 6132A cancer cells, the determining factor for successful adoptive T cell therapy was the isolation of a LYRE-TCR. In addition, FIG. 8 shows that successive treatment starting with the LYRE-TCR-transduced CD4⁺ T cells and injecting the CARE-TCR-transduced CD8⁺ T cells at a later time point can also be curative. Therefore, isolation and generation of a LYRE-TCR has priority over the CARE-TCR. However, both TCR-transduced T cell products are essential for eradication of large and long-established solid tumors.

### H. Glossary

APC: Antigen Presenting Cell. An APC expresses both MHC class I and class II molecules on the cell surface.

CARE-TCR: Cancer Cell Recognizing TCR. A CARE-TCR recognizes MHC class I restricted antigens on cancer cells.

CD4⁺ T cell: Cluster of Differentiation 4 expressing T cell. CD4⁺ T cells recognize MHC class II restricted antigens and are the source for isolation of CD4⁺ TCRs.

CD8⁺ T cell: Cluster of Differentiation 8 expressing T cell. CD8⁺ T cells recognize MHC class I restricted antigens and are the source for isolation of CD8⁺ TCRs.

CIB: Conditionally Immortalized autologous B cell. CIBs are a source for patient-derived autologous APCs.

DC: Dendritic Cell. DCs are a specific type of APCs.

LYRE-TCR: Lysate Recognizing TCR. A LYRE-TCR recognizes MHC class II restricted antigens that are processed and presented from lysates of cancer cells or tumor tissue.

MHC Major Histocompatibility Complex: MHCs are molecule complexes on the cell surface that present antigen and can be recognized by T cells. MHCs are divided into class I and class II.

NeoAg: Neoantigen. Antigens derived from nsSNVs that harbor neoepitopes and can be recognized by the adaptive immune system.

nsSNV: non-synonymous Single Nucleotide Variant. A single nucleotide substitution that leads to a mutated codon causing an amino acid exchange.

PBL: Peripheral Blood. Flowing, circulating blood of the body.

PBMC: Peripheral Blood Mononuclear Cell. Cells in the PBL that have a round nucleus. PBMCs consist of lymphocytes and monocytes.

SCC: Surrogate Cancer Cell. A CIB that expresses mutant neoantigens.

SNC: Surrogate Normal Cell. A CIB that expresses normal self-antigens.

TCR: T Cell Receptor. A receptor on the surface of T cells needed to recognize MHC class I or II restricted antigens.

TIL: Tumor Infiltrating Lymphocyte. Lymphocytes that left the blood circulation and migrated towards a tumor.

TNM: Tandem Normal Minigene. A vector construct of ten tandem normal minigenes. One minigenes encodes for a 25 amino acid long self-epitope.

TMM: Tandem Mutant Minigene. A vector construct of ten tandem mutant minigenes. One minigenes encodes for a 25 amino acid long mutant neoepitope with the mutated codon at the center position.

### Example 2: Cancers Harbor Mutation-Specific CD4 TCRs which Reprogram, Destroy and Arrest the Tumor after Adoptive Therapy

Cancers grow progressively eventually killing the host even when cancer-specific T cells infiltrate the tumor. Multiple mechanisms for the failure of these T cells have been observed. Reversing these failures has been difficult and current studies analyze mainly CD8+ T cells. Here the inventors examined whether hosts failing to control their cancers harbor cancer-specific T cell receptors on CD4⁺ T cells (CD4TCRs) that can be used successfully for TCR-T cell therapy. Among the numerous potential targets, the inventors focused on an immunodominant antigen deriving from a driver mutation essential for tumor growth and survival with loss of heterozygosity. This autochthonous antigen is only presented by the stroma of the MHC class-II negative cancer cells. Finding numerous T cell receptors, the inventors focused on 3 TCRs characterized by genetic convergence in 14 different T cell clones. When used for adoptive transfer of TCR-engineered T cells, each of the TCRs was similarly capable of destroying and permanently arresting large established solid tumors. CD4⁺ T cells with a TCR specific for an irrelevant mutation similarly infiltrated the cancer but had no effect. Only the cancer-specific TCR-treated tumor had tumor-associated macrophages reprogrammed to express nitric oxide and cancer cells that were arrested while cleaving caspase 3. Permanent arrest of cancer cell growth in vivo was reversible after tumor explanation in vitro. Thus, individuals with progressive cancers harbor therapeutically effective CD4TCRs that can be used for adoptive transfer of TCR-T cells.

Cancer is caused by somatic, cancer-specific mutations that are found in all types of cancers (1-3). Many of these somatic mutations are caused by non-synonymous single nucleotide variants and are potent tumor-specific mutant antigens (neoantigens) which can be targeted by adoptive transfer of neoantigen-specific T cells (4). Recent clinical data in patients treated with immune checkpoint inhibitors or by transfer of tumor infiltrating lymphocytes (TIL), support the long-standing notion that targeting neoantigens can be very effective (5-7). Unfortunately, such immunotherapies can achieve long-term survival only in a fraction of patients in certain types of cancers, and relapse is still common (8-9). The issue with both of these therapies may be that they rely on reverting the endogenous tumor-specific T cells into an active state, and that these reactivated T cells may quickly return to an inactive state once re-exposed to the tumor microenvironment (10,11). Thus, reactivating, invigorating and/or expanding dysfunctional cancer-specific TILs remains challenging.

An alternative approach would be “removing” the T cell receptors (TCRs) from the dysfunctional, cancer-specific T cell and transferring the TCR into healthy peripheral blood T cells for adoptive transfer (12,13). Indeed, adoptive T cell transfer of neoantigen-specific, TCR-engineered peripheral T cells eliminated large, established solid tumors (14). However, success depended on artificially high expression of the targeted neoantigen. When treating solid tumors with autochthonous, unmanipulated neoantigen expression, tumors regularly escaped as antigen-loss variants (14).

Dysfunction and tumor escape have been extensively studied in relation to CD8⁺ T cells and the antigens they recognize (15-17). So far, CD4⁺ T cells as effectors are relatively understudied even though clinical data suggest their potential in immunotherapies (18-20). Although many human malignancies are HLA class II-negative, CD4⁺ T cells nevertheless fulfill essential functions by recognizing cancer antigens indirectly during the induction phase on DCs in lymphoid organs. Thereby, help is provided providing to CD8⁺ T cells to eliminate cancer cells (21) not only during induction but also during the effector phase in the tumor stroma (22,23). In addition, CD4⁺ T cells may recognize antigens that are retained during tumor progression in highly malignant variants (24).

The inventors used the syngeneic, UV-induced mouse cancer cell model 6132A (25) to explore the therapeutic efficacy of neoantigen-specific TCRs isolated from CD4⁺ T cells (CD4TCR) from progressively growing tumors. The inventors had previously identified a 6132A-specific L47H mutation in the ribosomal protein L9 resulting in the I-E^{k}-restricted immunodominant neoantigen mL9 (4). This neoantigen is essential for growth and survival and lost its wild type L9 allele leading to loss of heterozygosity (26). This could therefore be an ideal target for T cell therapy.

Here the inventors show that adoptive transfer of T cells engineered to express mL9-specific CD4TCRs selected from tumor bearing mice destroyed and permanently arrested aggressively growing cancers targeting the original autochthonous unmanipulated neoantigen. Neither T cell exhaustion nor antigen loss, both hallmarks of cancer escape, were observed.

### A. Results

1. Tumor Bearers Respond with Multiple CD4⁺ T Cell Clonotypes to the Immunodominant Neoantigen

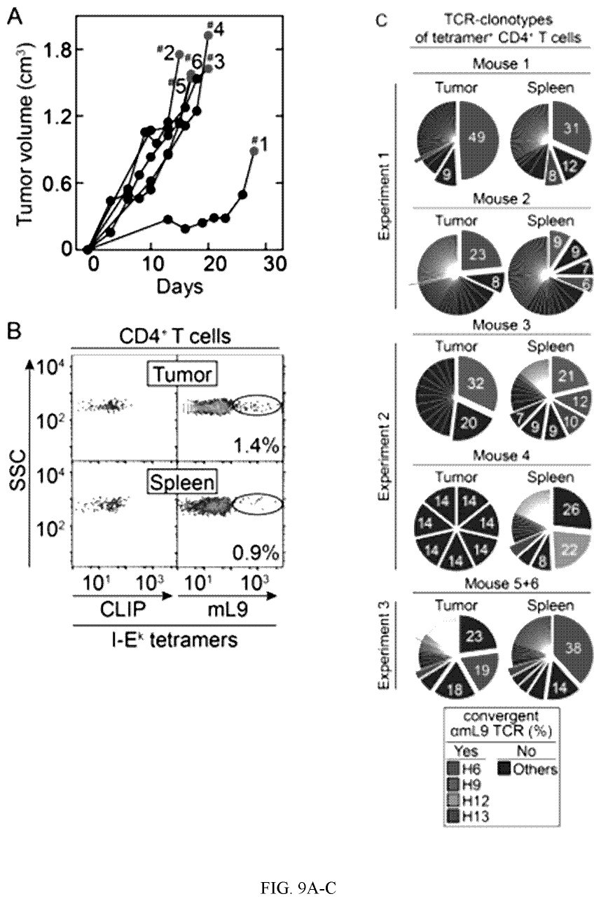

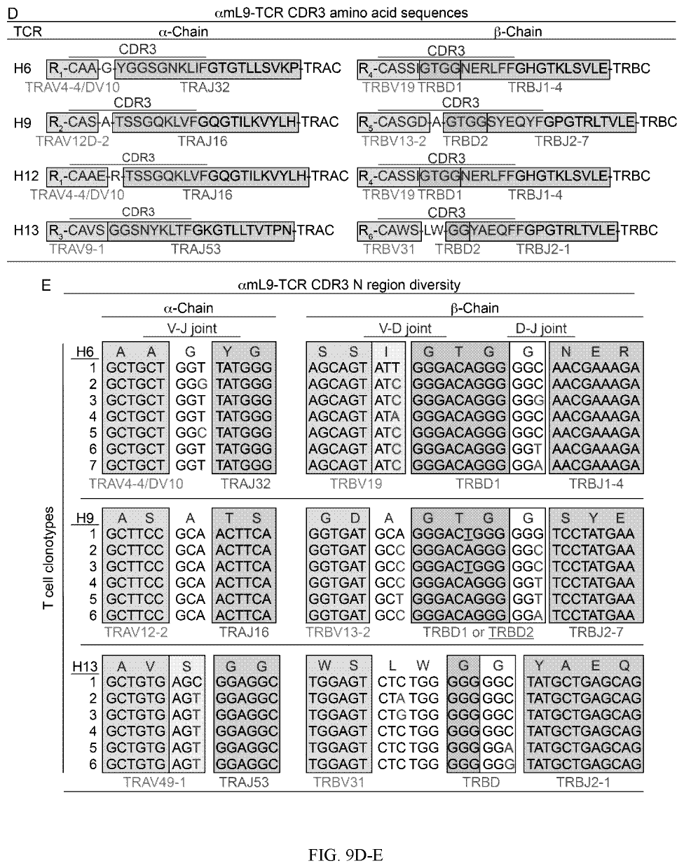

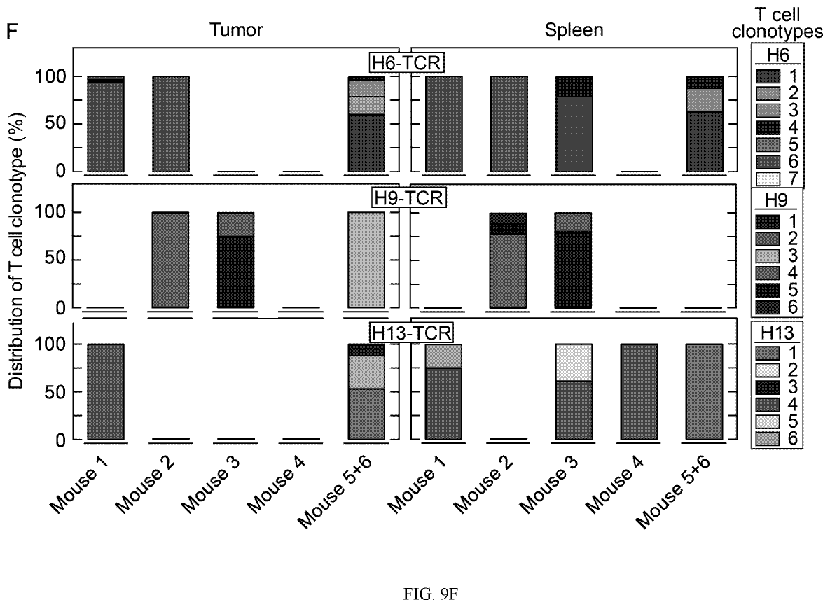

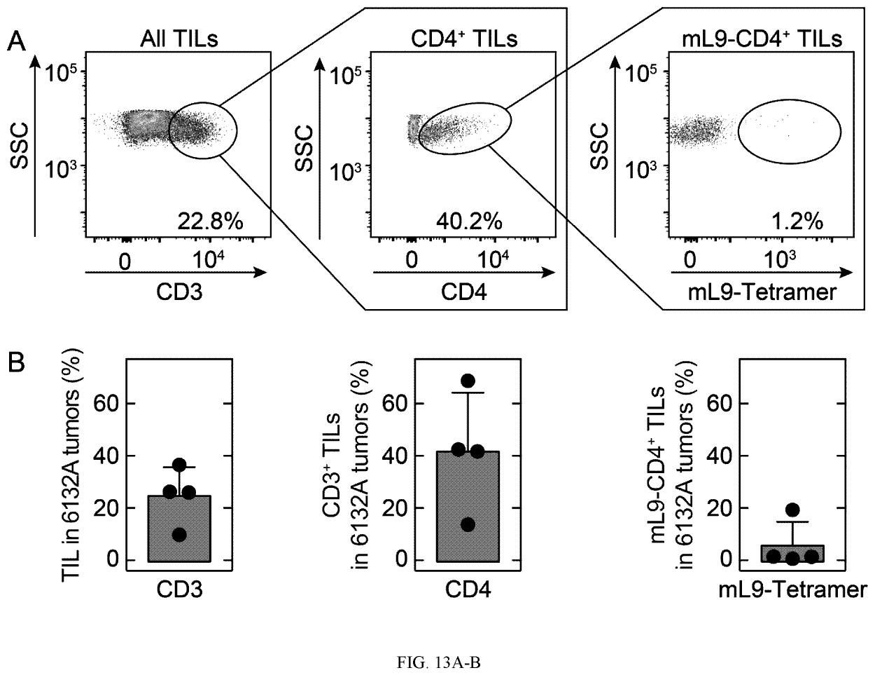

Tumors and spleens were isolated from normal immunocompetent C3H/HeN mice bearing aggressively growing 6132A tumors averaging >1 cm in diameters and established for over 2 weeks (FIG. 9A). Such mice would eventually die due to the high tumor burden. Nevertheless, these tumors were highly infiltrated and mice responded with T cells specific for the mL9 neoantigen (FIGS. 9B and 13). The inventors then performed single cell TCR sequencing with mL9-tetramer⁺ CD4⁺ T cells. FIG. 9C shows the relative frequencies of TCRs. On average, the inventors obtained 162 T cells harboring 45 different TCRs from the tumor sample and 202 T cells harboring 55 different TCRs from the spleen sample (Extended Data Table 1). The aim was to determine whether these CD4TCRs could be used for adoptive transfer of TCR-engineered T cells even though the TCRs were isolated from mice with progressively growing 6132A tumors and an immune response that failed to prevent a lethal outcome.

### 2. Genetic Convergence of Multiple T Cell Clones

To choose from around 50 different TCRs the single one that can realistically be used in a given individual for adoptive transfer of TCR-engineered T cells, the inventors chose the two most common TCRs found in tumor or spleen of the individual tumor-bearing mice, H6 and H9 (FIG. 9C). These two TCRs have completely different CDR3 amino acid sequences. The inventors also included TCR H12 for therapy studies because it uses the TCRbeta-chain CDR3 sequence of H6 with a TCRalpha-chain representing a recombination between H6 V region and H9 J region sequences (FIG. 9D). Furthermore, each of the TCRs H6 and H9 were generated by multiple different T cell clones as determined by different N-nucleotides between the V(D)J joints (FIG. 9E). Seven different T cell clones in at least four different mice developed the H6-TCR while six different T cell clones in at least three different mice developed the H9-TCR (FIG. 9F). This indicates that multiple T cell clones responded to the progressively growing 6132A tumor in different mice in separate experiments by convergent recombination on the same TCR CDR3 amino acid sequence to recognize the mL9 neoantigen. Another TCR (H13) with yet again completely different TCRalpha and TCRbeta CDR3 sequences, was less frequent than H6, H9, and H12, but also showed convergent recombination by six different T cell clonotypes found in at least four mice.

3. CD4⁺ TCRs from Progressive Tumors Cause Tumor Destruction Followed by Growth Arrest Upon Adoptive Transfer

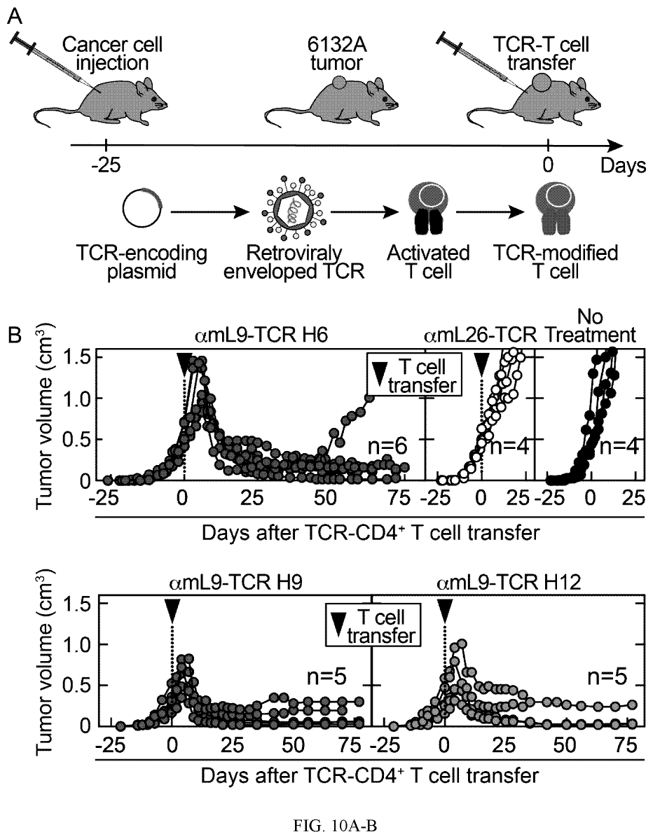

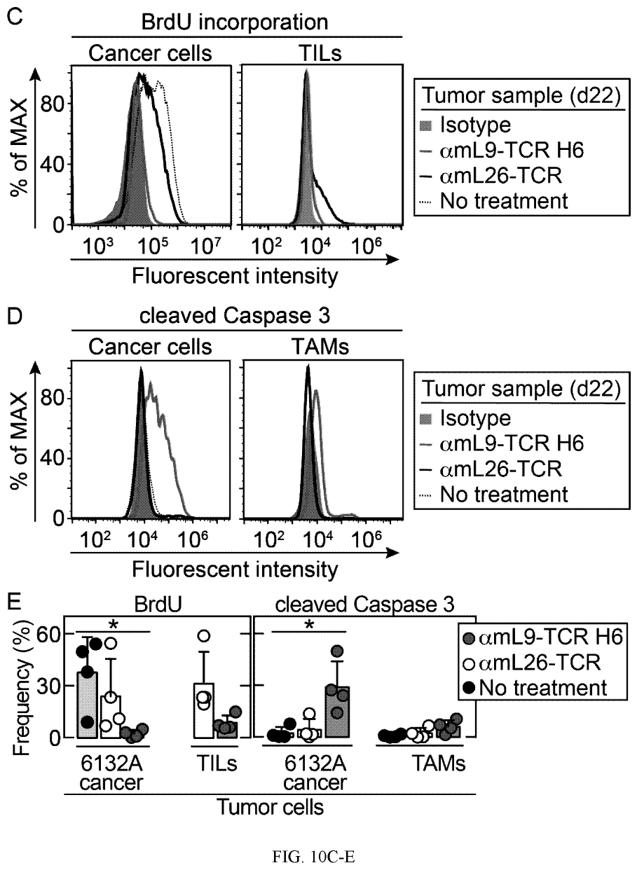

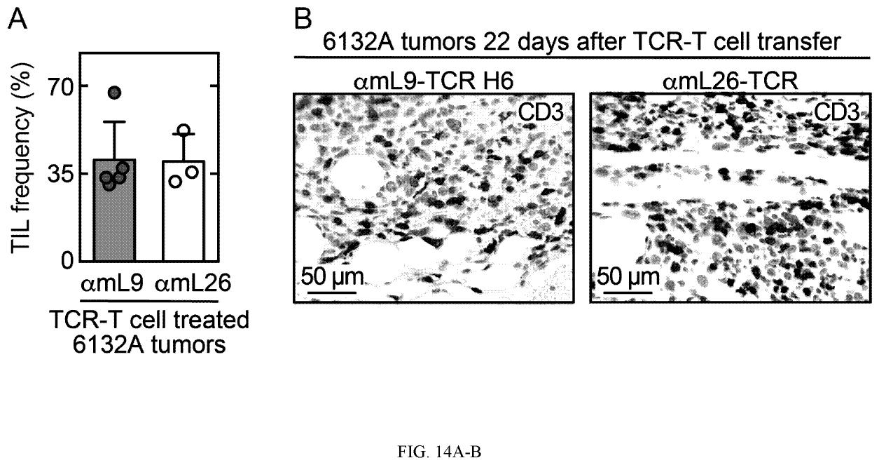

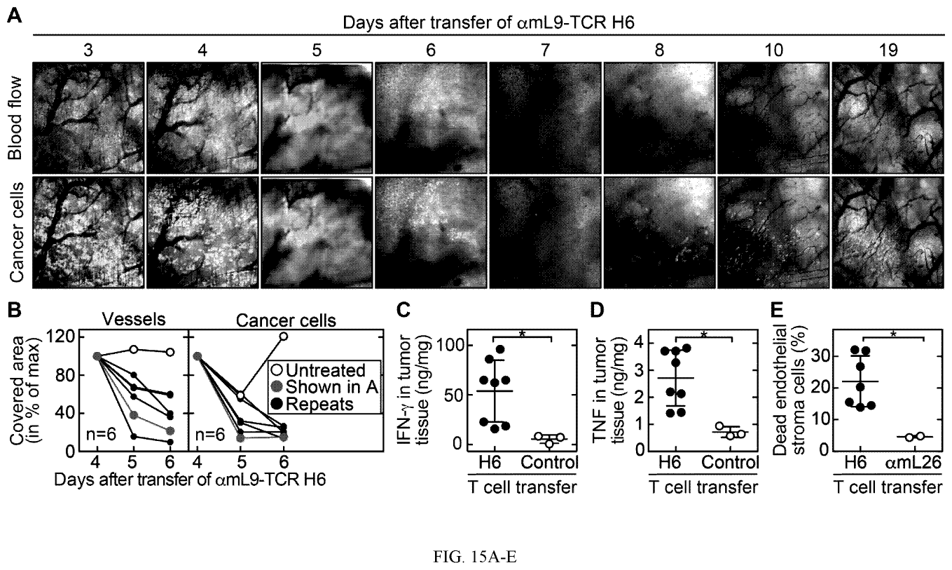

TCRs H6, H9 and H12 were cloned into retroviral vectors and transduced into splenic T cells from C3H CD8^{-/-} mice. These TCR-engineered CD4⁺ T cells were adoptively transferred in C3H Rag^{-/-} mice bearing solid 6132A tumors at least 1 cm diameter and established for 21 to 25 days (FIG. 10A). T cells destroyed large established tumors within 10 days after transfer (FIG. 10B). After a few days, tumors often turned dark-red and then collapsed. Transfer of a CD4TCR targeting an irrelevant mutant ribosomal protein mL26 (found in another UV-induced C3H tumor, 6139B, (26)) had no effects. The 6132A tumors in these mice progressed like those in the untreated controls (FIG. 10B). Nevertheless, tumors treated with the completely ineffective αmL26-TCRs were similarly infiltrated as those treated with the therapeutically effective αmL9-specific TCR (FIGS. 14A and 14B) indicating the effects were solely antigen-specific. To further analyze how the mL9-specific CD4⁺ T cells cause tumor destruction, the inventors used longitudinal confocal microscopy (27) to follow up areas of vasculature, blood flow, and cancer cells in H6-T cell treated 6132A tumors for almost three weeks (FIG. 15A). By day 6 after ATT, blood flow disappeared and the area covered by vessels regressed about 50%, while the area covered by cancer cells regressed on average by 70% compared to day 4 after ATT, which was not seen in untreated tumors (FIG. 15B). Starting around day 10 after ATT, the opaqueness of the windows which followed the acute phase of the tissue destruction cleared and patches of blood flow, vasculature structure and cancer cells became visible in the window behind the smaller tumor remaining in growth arrest. The inventors reported in an earlier study that high levels of IFN-gamma can cause regression of tumor vasculature and ischemic necrosis (28). Therefore, 6132A tumor tissue was analyzed for IFN-gamma and TNF levels on day 6, 7 and 8 after ATT of H6-T cells. As controls, 6132A tumor-bearing C3H Rag^{-/-} mice received either no ATT or were treated with mL26-specific T cells. Tumors from H6 treated mice had significantly higher IFN-gamma and TNF values compared to the control tumors (FIGS. 15C and 15D). Consistently with an earlier study, a significant part of endothelial cells (CD31⁺ and CD146⁺) was found dead in H6-treated 6132A tumors compared to the controls (FIG. 15E).

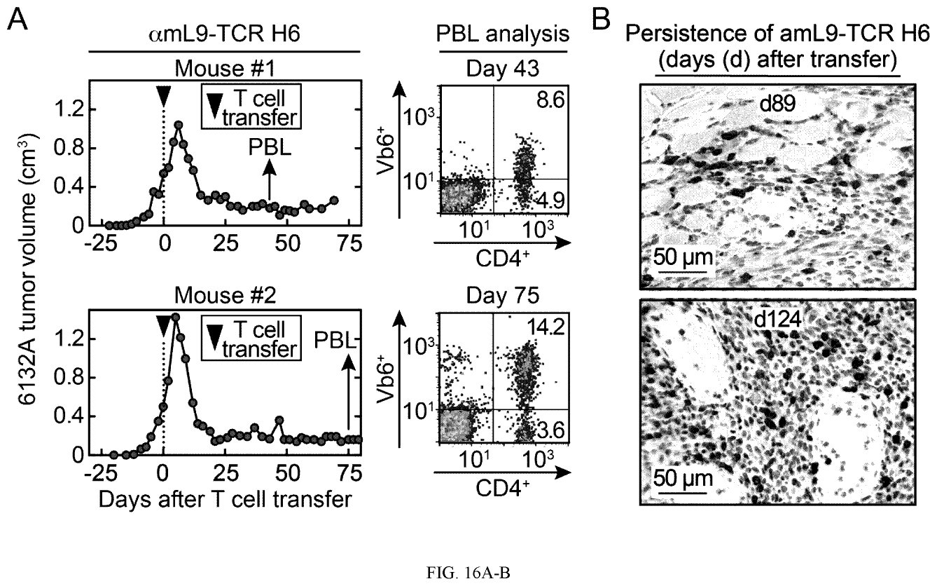

After the bulk of the tumor mass had been destroyed, tumors persisted afterwards at a much smaller sizes during the entire observation period (at least 75 d up to several months) (FIG. 10B). Thus, these tumors were stably arrested but never completely eradicated. T cells could be detected in peripheral blood and in the remaining small tumor for more than two months (FIG. 16A). Even at the longest observation time points no decrease in the intensity of CD4⁺ T cell infiltration was notable (FIG. 16B).

To analyze the continued tumor arrest, the inventors injected mice with BrdU for three consecutive days. The inventors found that proliferation of 6132A cancer treated with the therapeutic H6 TCR had ceased almost completely (FIG. 10C). Nevertheless, a large fraction of 6132A cancer cells treated with the H6 TCR showed cleavage of caspase 3 determined by flow cytometry (FIG. 10D). This effect was exclusively dependent on using the mL9-specific H6 TCR (FIGS. 10D and 10E). Cleavage of caspase 3 is usually associated with cell death and apoptosis but can also be reversible (29). Indeed, 6132A tumors that were arrested for 21 days could be readapted in vitro as cell line by removing the tumor from the αmL9-TCR H6-treated host. Since cleaved caspase 3 can be associated with DNA instability (30), the inventors whole-exome and RNA-sequenced (Extended Data Table 2) 6132A tumor cells readapted in vitro from the H6-treated tumor. For comparison, 6132A readapted from untreated or αmL26-TCR treated tumors that rapidly grew out were also whole-exome and RNA-sequenced. The expression of nsSNV by these three cells lines was virtually indistinguishable, thereby showing that growth arrest did not lead to any notable acquisition of additional mutations.

### 4. Stroma Recognition is Essential and Sufficient for Tumor Destruction

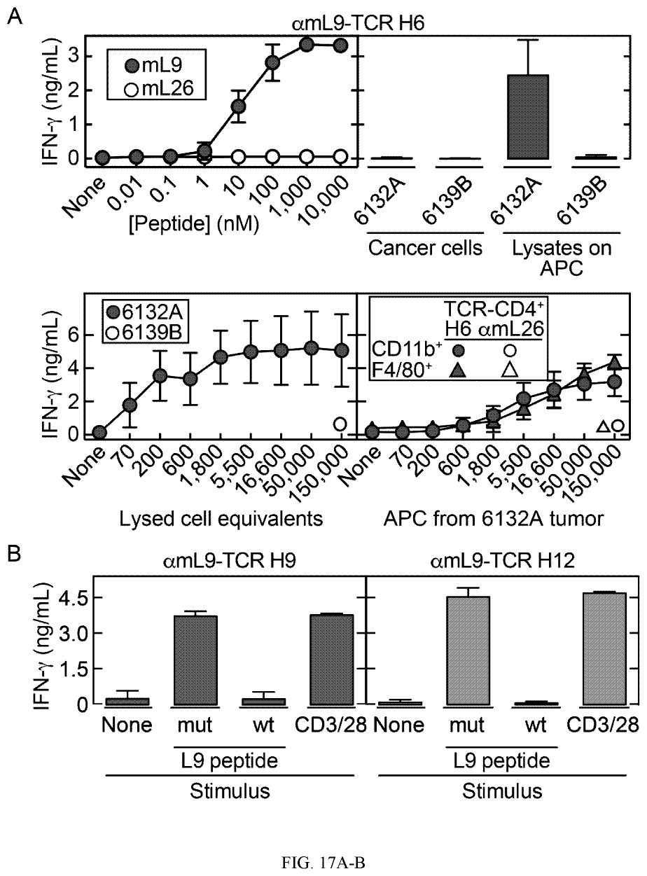

H6-T cells specifically recognize the mL9 but not mL26 peptide presented by C3H/HeN spleen cells in vitro (FIG. 17A, left). Interestingly, 6132A cancer cells are not directly recognized. Instead, lysates of 6132A but not 6139B when cultured together with spleen cells are recognized. CD11b⁺ cells isolated from 6132A tumors are equally well recognized as lysates from 6132A cancer cells (FIG. 17A, right). Similar levels of IFN-gamma secreted by H6-T cells were also elicited by F4/80⁺6132A tumor-associated macrophages (TAMs) indicating that stroma recognition did not depend entirely on dendritic cells. The same mL9-specificity was also observed with H9- and H12-T cells (FIG. 17B).

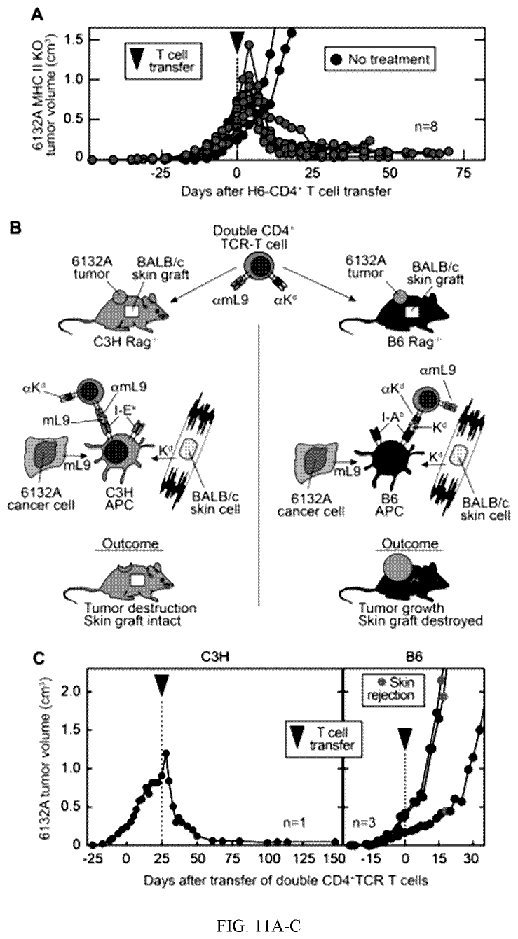

6132A cancer cells do not upregulate MHC class II molecules after exposure to IFN-gamma (31) but other mechanisms in vivo could have this effect. Therefore, the inventors knocked out the beta chain of the I-E MHC class II molecule and determined whether the effect of CD4⁺ T cells depended solely on stroma recognition. FIG. 11A shows that H6-T cells were able to cause tumor destruction followed by growth arrest even when I-E^{k} is knocked out in 6132A cancer cells. In addition, H6-T cells depended on host I-E^{k} expression and were only effective when transferred into C3H but not into B6 mice (FIGS. 11B and 11C). To verify the function of the transferred T cells, the inventors made use of TCR75-transgenic CD4⁺ T cells as recipients for the H6-TCR. The TCR75 recognizes an H-2K^{d} derived epitope presented on I-A^{b} and therefore causes BALB/c skin graft rejection in B6 mice (32). The endogenous TCR75 of the adoptively transferred double TCR75/H6-expressing T cells rejected full-thickness BALB/c skin grafts only in B6 but not in C3H mice showing that adoptively transferred CD4⁺ T cells are effective only when the appropriate stroma is recognized.

### 5. Antigen-Specific Reprogramming of TAMs to Produce NO

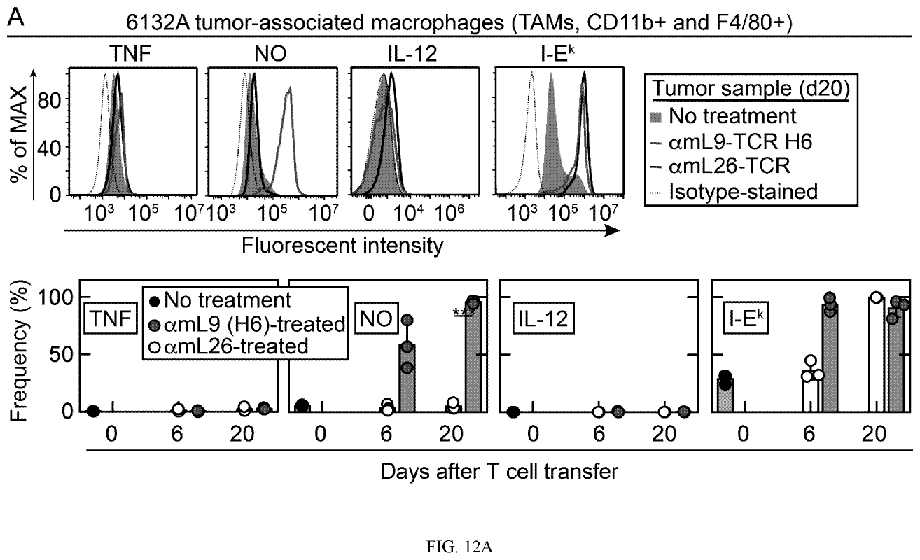

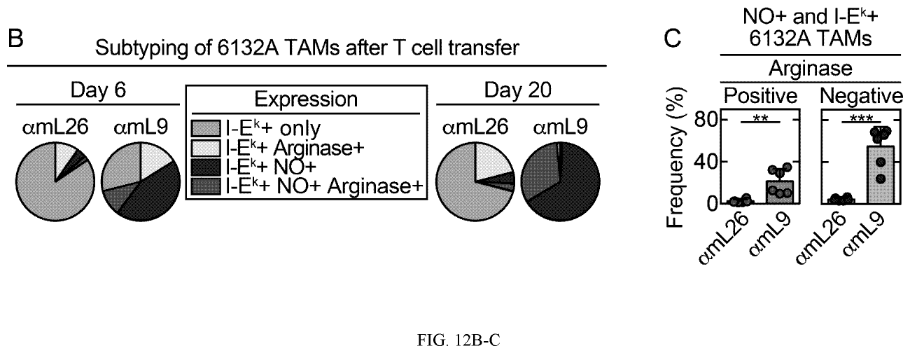

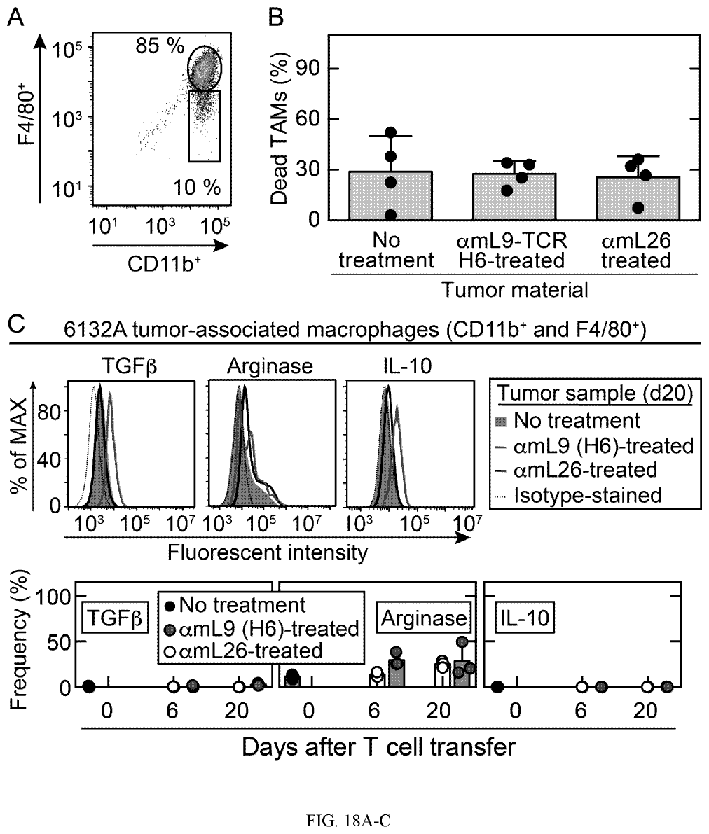

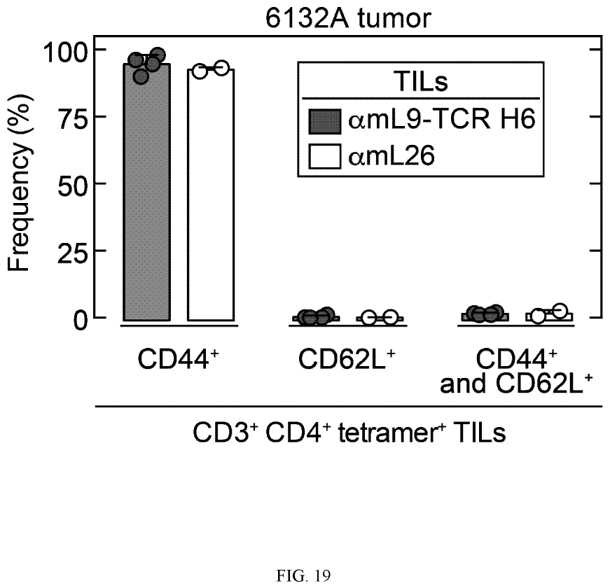

Since the only required interaction for tumor destruction and growth arrest seems to be between stroma and CD4⁺ T cells, the inventors examined further the interaction between TAMs and CD4⁺ T cells. More than 80% of all CD11b⁺ cells in the 6132A microenvironment are F4/80⁺ cells (FIG. 18A). Stroma recognition of the TAMs by the mL9-specific CD4TCRs was not associated with an increased death rate of TAMs since the number of non-viable TAMs did not differ significantly between untreated, anti-mL9 or anti-mL26 TCR-treated tumors (FIG. 18B). Next, the inventors examined whether TAMs changed their phenotype in response to mL9-specific treatment. Tumors grown in C3H Rag^{-/-} mice were taken out at day 0, 6 and after ATT of either αmL9- or αmL26-T cells and TAMs were analyzed by FACS for the M2-type proteins TGFβ, Arginase and IL-10 (FIG. 18C). The TAMs showed expression of arginase but not TGFβ or IL-10 and no significant changes in either of these cytokines were found by day 20 in both αmL9- and αmL26-T cell treated tumors. The inventors then analyzed M1-type proteins TNF, NO, IL-12 and MHC class II I-E^{k} (FIG. 12A). While the inventors did not observe differences in TNF and IL-12, the inventors found significant induction of NO expression in almost all TAMs by day 20 when tumors were treated with αmL9-T cells but not when treated with αmL26-T cells. Surprisingly, the inventors also observed upregulation of I-E^{k} in almost all TAMs in both tumor samples by day 20. However, analyzing the I-E^{k}-expressing TAM population showed that only αmL9-treated samples consisted mostly solely of NO producing TAMs while this cell population was almost completely absent in αmL26-treated samples (FIGS. 12B and 12C) despite the CD44⁺ and CD62L⁺ T-effector phenotype of both TIL populations (FIG. 19) indicating again the antigen-specificity of the CD4TCR treatment.

### B. Discussion

This is the first demonstration that certain CD4TCRs isolated from hosts with progressive cancer can be used for adoptive transfer to destroy and then permanently arrest the solid tumors.

The 6132A tumor model had been derived from the autochthonous primary tumor, never been serially passaged in vitro for more than 4 weeks, and never passaged in vivo, since immunoselection may occur even after a single transplantation into a naïve fully immunocompetent host (33-35). In addition, the inventors only treated large and established tumors. Thus, the 6132A model may mirror a typical primary cancer with antigens expressed as autochthonous unmanipulated targets as they occur in a human patient (36). Sequencing analyses revealed 1,687 potential mutant targets to choose from for therapy. While using multiple TCRs simultaneously targeting multiple neoantigens can also be effective to prevent escape (37,38), the number of different TCRs that can be realistically used in a given patient is very limited. It is therefore highly significant that a single type of CD4TCR targeting a single cancer-specific neoantigen sufficed for therapy.

The CD4TCRs were selected to recognize the autochthonous immunodominant mL9 neoantigen. Recombinational convergence of 20 different T cell clonotypes on four TCRs indicated the significance of this mutation as a target. The convergence of the T cell clones resulted from antigen-driven selection by the tumor bearers (39) and did not reflect the clonotypic landscape of the naïve T-cell repertoire (40) since different but genetically identical mice selected distinctly different clonotypes to respond to the immunodominant antigen.

Most human cancers do not express MHC class II and do not allow for direct recognition by CD4⁺ T cells, as observed in the tumor models (31) even though melanoma represents a notable exception (41-43). Nevertheless, adoptive transfer of CD4⁺ T cells has been shown to eradicate disseminated Friend virus-induced erythroleukemia and these cancer cells were found to be MHC class II negative (44). A decrease in targeted lesions and growth control of the persistent cancer has also been achieved in patients after transfer of in vitro-expanded mutation-specific CD4⁺ tumor-infiltrating T cell populations (18,19). In both, the inventors' experimental model and clinical studies, cancer cells were MHC class-II negative, yet persistence of the mutation-specific CD4⁺ T cells could be demonstrated. The need of CD4+ T cells for successful immunotherapy is becoming more recognized (45-47) and efforts are being made to improve neoantigen prediction (48).

The T cells in the inventors' model could only recognize TAMs and this effector mechanism can be subjected to spatial restriction which can lead to escape of antigen loss variants (49). However, targeting mutant ribosomal proteins maybe ideal targets for cancer therapy by CD4⁺ T cells. These targets are not only mutant tumor-suppressors but the mutant gene maintains an essential function required for cell survival, tumor growth and cannot be lost because of loss of the wild type gene (26,50,51). This loss of heterozygosity (LOH) may be a reason why the inventors did not observe relapse. LOH is increasingly recognized to provide a widespread class of potential cancer targets (52,53) and a novel paradigm for cancer therapy (54).

Reversing exhaustion and expanding cancer-specific T cells in cancer bearing individuals are the goals of numerous current studies using immune checkpoint blockade or other measures. Interestingly, clinical observations show that exhaustion signatures are preferentially associated with the CD8⁺ T cell compartment (17,55,56) and that the TIL repertoire could be replaced with new T cell clones instead of reversing the dysfunctional ones (57). This approach bypasses the need for correcting any insufficiencies or dysfunctions of TILs. Instead, the inventors used neoantigen-specific TCRs from TILs for TCR-engineering of healthy peripheral blood T cells. Each of these different T cell clonotypes redirected to express the same cancer-specific TCR has its own degree of activation and proliferation capacity only limited by the Hayflick rule (58,59). Interestingly, the mL9- and mL26-specific T cells used in the model both infiltrated tumors similarly and showed effector memory phenotypes (T_{EM}), but only mL9-specific T cells showed lower rate of proliferation. This indicates a smaller likelihood of exhaustion and is consistent with other studies showing that the half-life of some CD4⁺ T_{EM} may be much longer than previously assumed (60).

The tumor microenvironment is widely considered to be immuno-suppressive (61) and tumor promoting (62), and is therefore a barrier for effective T cell therapy. The approach described herein overcomes this problem and gives evidence for the concept that this immunosuppressive, tumor-promoting microenvironment can be destroyed and then reprogrammed by neoantigen-specific T cells. Longitudinal in vivo imaging showed the destruction of tumor vessels followed by growth arrests of cancer cells. These effects are consistent with a previous report on the effects of IFN-gamma on tumor vasculature (63) where artificially high amounts of IFN-gamma were induced inside tumor tissue but it had remained unknown whether such high IFN-gamma values could be reached by targeting neoantigens. In addition, IFN-gamma and TNF are associated with growth arrest in cancer cells (64) and both cytokines were secreted at high levels only after mL9-specific T cell transfer. Remarkably, the inventors observed stromal reprogramming with almost 100% of the TAMs expressing nitric oxide (NO) when the antigen-specific TCR was used versus <5% for the control TCR. Previous studies showed that CD4⁺ T cells producing IFN-gamma can induce the activation of nitric oxide synthase in TAMs (65,66) and thereby prevented outgrowth of cancer cell inocula. The stromal reprogramming was associated with a complete growth arrest of the cancer cells rather than active killing of the stromal cells as it had previously been reported following CD8+ T cell transfer 67,68). Importantly, the cytostatic effect of NO on cancer cells was already discovered decades ago and was found to be reversible (69). The inventors were also able to readapt cancer cells in vitro after complete growth arrest and cleavage of caspase-3 which is consistent with the effects of NO as a bifunctional regulator of apoptosis (70).

This proof of principle that cancers harbor therapeutically effective neoantigen-specific CD4TCRs shows an alternative approach to cancer immunotherapy which does not lead to cancer eradication but long-term growth arrest without relapse. Together with long-lasting T cell activity and no signs of exhaustion, this strategy could become beneficial to most if not all cancer patients.

### C. Tables

Extended Data Table 1. Distribution of mL9-tetramer
sorted CD4⁺ T cells and associated
TCR clonotypes by amino acid sequence among all mice.

| | Tumor | | Spleen | |
|---|---|---|---|---|
| | Number of | Number of | Number of | Number of |
| Mouse | T cells | TCRs | T cells | TCRs |
| 1 | 63 | 25 | 104 | 41 |
| 2 | 175 | 74 | 99 | 54 |
| 3 | 25 | 14 | 285 | 55 |
| 4 | 7 | 7 | 372 | 83 |
| 5 + 6 | 543 | 108 | 149 | 42 |
| Average | 162.6 | 45.6 | 201.8 | 55 |

Extended Data Table 2. Analysis of expressed nsSNVs in
reisolated progressing or arrested 6132A tumors.

| 6132A | | mL9 expression | Expressed nsSNV |
|---|---|---|---|
| reisolate^{A} | Treatment | (RNA FPKM) | (RNA FPKM ≥ 5) |
| #4718 | None | 548.493 | 1976 |
| #4719 | αmL9-TCR H6^{B} | 466.526 | 1951 |
| #4720 | αmL26^{B} | 619.572 | 1961 |

^{A}All 6132A reisolates were from mice injected with tumor cells 45 days earlier
^{B}T cell transfer was done 21 days after cancer cell injection

### D. Material and Methods

### 1. Mice

Both female and male mice were used in this study and were between 3 to 8 months old. Mice were euthanized when tumor sizes reached more than 2 cm³ or mice appeared hunched and weak. Littermates of the same sex were randomly assigned to experimental groups on the day of adoptive T cell transfer. Mice were bred and maintained in a specific pathogen-free barrier facility at The University of Chicago according to Institutional Animal Care and Use Committee (IACUC) guidelines. All animal experiments were approved by the IACUC of The University of Chicago. C3H/HeN and BALB/cAnN mice were obtained from Envigo (Huntingdon, Cambridgeshire, United Kingdom). C3H Rag2^{-/-} (C3H.129S6-Rag2^{tml1wa}) mice were obtained from Douglas Hanahan (University of California, San Francisco, CA, USA). C57BL/6 Rag1^{-/-} mice were purchased from the Jackson Laboratory (B6.129S7-Rag1^{tm1Mom}/J) C3H CD8^{-/-} (C3H.129S2-Cd8a^{tm1Mak}) mice were generated in house by crossing C3H/HeN mice with C57BL/6 CD8^{-/-} mice purchased from the Jackson Laboratory (B6.129S2-Cd8a^{tm1Mak}) and then backcrossed with C3H/HeN for 20 generations. TCR75 mice (32) (Tg(CD2-Tcra,-Tcrb)75Bucy) were obtained from Anita Chong (University of Chicago, Chicago, IL, USA) and were crossed with C57BL/6 Rag1^{-/-} mice from the Jackson Laboratory (B6.129S7-Rag1^{tm1Mom}/J) to generate TCR75 Rag1^{-/-} mice which solely produced CD4⁺ TCR75tg T cells (B6.129S7-Rag1^{tm1Mom} Tg(CD2-Tcra,-Tcrb)75Bucy). Spleen of C3H CD8- and TCR75 Rag1^{-/-} mice were used as T cell sources for TCR-engineering.

### 2. Cell Lines

6132A and 6139B cancer cell lines originated from UV-treated C3H/HeN mice and were generated in the inventors' laboratory together with heart-lung fibroblasts as normal tissue control (25). Autochthonous tumor was minced and fragments were used to establish uncloned primary cultures of 6132A cancer cells. These primary tumor cell cultures were only minimally expanded, and used for cell culture experiments and tumor induction in vivo. The 6132A-ECFP was generated by using retroviral transduction with the pMFG-ECFP vector as described before (27). 6132A-Cerulean was also described before (71). Knockout of the H2-Eb1 gene results into I-E beta chain loss and therefore loss of MHC class II expression (72). The 6132A-H2-Eb1 knockout cell line was generated using CRISPR-Cas9. Single guide (sg) RNAs targeting exon 1 of the murine C3H H2-Eb1 gene were designed using the sg RNA design tool from the Broad Institute (73). The corresponding sense and antisense DNA oligomers (IDT, Coralville, IA, USA) were compared to other publications that also targeted H2-Eb1 to generate murine MHC class II knockout cancer cell lines (72). The DNA oligomers were annealed and cloned over an BbsI side into PX458 as described (74). The sg RNA 5′-AGGAGACACGAGAGTCAGAG-3′ was successfully used to generate 6132A-H2-Eb1—cancer cells which were verified by sanger-sequencing to have an indel and frameshift in exon 1. Cancer cells were maintained in DMEM supplemented with 5% FBS (Gemini Bio-Products) and 2 mM L-glutamine (Life Technologies, Carlsbad, CA, USA) and cultured at 10% C02 in a 37° C. dry incubator. Plat-E packaging cells (75) used for TCR gene transfer were maintained in DMEM supplemented with 10% FBS, 2 mM L-glutamine, 1 μg/mL Puromycin and 1 mg/mL Blasticidin (Invivogen, San Diego, CA, USA) and cultured at 5% C02 in a 37° C. dry incubator. Before use, tumor cell lines were authenticated by sequencing and/or co-culture with antigen-specific T cells and by morphology. All cell lines were shortly passaged after thawing of the initial frozen stock to generate master cell banks. Working batches were passaged no longer than 4 weeks.

### 3. Cell Sorting, Single Cell Sequencing and Isolation of TCR Genes

After harvesting tumor and spleen tissue between day 18 and 28 after injection of cancer cell fragments, single cell suspensions were prepared and stained for Sytox Blue (Helix NP Blue, Biolegend, San Diego, CA, USA, life/dead stain), CD3, CD4 and tetramer respectively before cell sorting of tetramer binding CD4⁺ T cells was done (FACSArialI, BD Bioscience, Franklin Lakes, NJ, USA). Afterwards, single cell analysis was performed with the Chromium controller (10× Genomics, Pleasanton, CA, USA). The 10× Genomics single cell 5′ dual index platform was used to generate RNA-expression and TCR-libraries following manufacture protocol. Next generation sequencing was performed at the University of Chicago Genomics facility using NovaSeq 6000 (Illumina, San Diego, CA, USA). Obtained TCR sequences were ordered, codon optimized (GeneArt, Thermo Fisher Scientific, Waltham, MA, USA) and integrated into the pMP71 vector using NotI and EcoRI flanked restriction sides as described (76). The T cell clone specific for mL26 against 6139B has been characterized before (26). This TCR sequence TRAV13-CAMVTGANTGKLTF-TRAJ52 and TRBV1-CTCSAHNNQAPLF-TRBD1/TRBJ5 was obtained by 5′-RACE-PCR (TaKaRa, Kusatsu, Japan) following manufactures protocol, codon optimized (GeneArt, Thermo Fisher Scientific, Waltham, MA, USA) and also integrated into the pMP71 vector.

### 4. TCR-Engineering and T Cell Culture

TCR-engineering was conducted as previously described (77). Plat-E packaging cells were transfected with pMP71-H6, -H9, -H12 or pMP71-αmL26 by calcium phosphate precipitation. 42 h after transfection, virus supernatant was removed and filtrated through a 0.45 μm syringe filter (VWR, Radnor, PA, USA). Spleens were isolated and erythrocytes were lysed for 3 min with 0.017 M TRIS, 0.14 M ammonium chloride (both Sigma-Aldrich, St. Louis, MO, USA). Cells were cultured in complete medium containing Roswell Park Memorial Institute medium (RPMI 1640, Corning, Corning, NY, USA) 10% FBS (Gemini, Sacramento, CA, USA), 2 mM L-glutamine, 1 mM sodium pyruvate, 0.1 mM non-essential amino acids, 100 U/mL penicillin, 100 μg/mL streptomycin (all purchased from Life Technologies, Carlsbad, CA, USA), 50 μM 2-mercaptoethanol (Thermo Fisher Scientific, Waltham, MA, USA), 50 μg/mL gentamycin (VWR, Radnor, PA, USA) and were supplemented with U/mL recombinant human IL-2 (Peprotech, Rocky Hill, NJ, USA). The cell suspension was transferred into a 24-well plate (Greiner Bio-One, Kremsmuenster, Austria) coated with 1.4 μg/mL αCD3 (University of Chicago, Frank W. Fitch Monoclonal Antibody Facility, Clone 145-2C11.1) and 0.2 μg/mL αCD28 (Clone 37.51, Biolegend, San Diego, CA, USA) at a concentration of 3×10⁶ cells/mL. On the subsequent day, 0.5 mL of corresponding virus supernatant containing 8 μg/mL protamine sulfate (Sigma-Aldrich, St. Louis, MO, USA) was added per well and cells were spinoculated (800× g, 90 min, 32° C.). Overnight, a 12-well plate (Greiner Bio-One, Kremsmuenster, Austria) was coated with RetroNectin [12.5 μg/mL (TaKaRa)] and centrifuged with 1.5 mL virus supernatant (3000× g, 90 min, 4° C.) on the next day. The virus supernatants were removed and 5×10⁶ of CD4⁺ T cells in complete medium containing 40 U/mL IL-2 were transferred to the virus coated 12-well plate and followed by spinoculation (800× g, 90 min, 32° C.). Transduction rate was confirmed by flow cytometry using NovoCyte Quanteon (Agilent, Santa Clara, CA, USA) and T cells were used 3 days after transduction for adoptive transfer. TCR-engineered CD4⁺ T cells were maintained in complete medium with 40 U/mL IL-2 and used after 4 days for in vitro analyses respectively.

### 5. Treatment of Tumor-Bearing Mice

For generation of tumor bearing C3H/HeN wild type mice, 6132A-fragments were generated and injected s.c. as previously described (35). For treatment of established 6132A tumors, cancer cells were injected s.c. into the shaved back of C3H Rag2^{-/-} mice (1×10⁷ 6132A cancer cells). Tumor volumes were measured along 3 orthogonal axes, every 2-3 days and were calculated as (a×b×c)+2. T cells expressing the H6-, H9-, H12 or αmL26-TCR were injected i.p. The number of TCR⁺ T cells was calculated based on transduction rate (on average ˜30%) on the day of treatment prior to T cell transfer. Per recipient, 2×10⁶ TCR⁺ CD4⁺ T cells were injected. Mice were randomized into different treatment groups on the day of adoptive T cell transfer. Mice were euthanized when tumor sizes reached more than 2 cm³ or mice appeared hunched and weak due to high tumor burden.

6. Tumor Preparation and Isolation of CD11b⁺ and F4/80⁺ Cells

6132A tumors, either grown in C3H/HeN mice for the isolation of tetramer-binding CD4⁺ T cells or grown in C3H Rag2^{-/-} mice for isolation of APCs, were removed and single cell suspensions were generated by enzymatic digestion (78). Tumors were minced, 2 mg/mL Collagenase D and 100 U/mL DNAse I (both Roche, Indianapolis, IN, USA) were added and suspension was incubated for 20 min at 37° C. in RPMI on a horizontal shaker, following addition of trypsin in Hanks' Balance Salt Solution (HBSS, MP Biomedicals LLC, Solon, OH, USA) to a final concentration of 0.025% and cell suspension was incubated for additional 15 min at 37° C. on a horizontal shaker. Tumor cell suspension was filtered over a 40 μm cell strainer (Thermo Fisher Scientific, Waltham, MA, USA) and used subsequently. For the isolation of APCs, CD11b⁺ and F4/80⁺ cells were collected by magnetic cell sorting (Miltenyi, Bergisch Gladbach, Germany) following manufacturer's protocol. Successful isolation was confirmed by FACS before both cell populations were used for T cell stimulation.

### 7. Tumor Tissue Analysis

At day six, seven and eight after ATT, tumors were isolated and about 100 mg were homogenized using Polytron (Kinematica, Lucern, Swiss) and spun down. Supernatants were used for determination of cytokines by flow cytometry using Legendplex according to manufacture protocol (Biolegend, San Diego, CA, USA). For endothelial cell analysis, single cell suspension from tumor tissue was generated as described in “Tumor preparation”. Tumor single cell suspensions were analyzed for dead CD31V and CD146′ cell populations with Sytox Blue (Helix NP Blue, Biolegend, San Diego, CA, USA) by flow cytometry.

### 8. Antigen Presentation and T Cell Stimulation

T cells, transduced with the H6-, H9-, H12- or ∞mL26-TCR were cocultured for 24 h (77) to analyze antigen presentation by indicated cancer and stromal cells. In brief, 5×10⁴ TCR-transduced CD4⁺ T cells were added to 1×10⁵ cancer cells or stromal cells. For TCR independent stimulation, 8 μg/mL αCD3 (University of Chicago, Frank W. Fitch Monoclonal Antibody Facility, Clone 145-2C11.1) and 2 μg/mL αCD28 (Clone 37.51, Biolegend, San Diego, CA, USA) was used. In addition, antigen in form of cancer cell lysate cultured with CD11b⁺ cells isolated from spleen of C3H/HeN cells were performed as previously described (4,79). 6132A or 6139B cancer cells were adjusted to 1×10⁷ cells/mL in RPMI 1640 (Corning, Corning, NY, USA) before three cycles of freezing in liquid nitrogen and thawing at 37° C. were conducted. Cancer cell lysate was cultured together with 1×10⁵ CD11b⁺ cells for antigen presentation. In addition, spleen cells were also cultured with 26mer mL9 and mL26 peptides at various concentrations indicated in the figure legends. After 24 h, supernatants were removed and tested for IFN-γ concentrations by enzyme-linked immunosorbent assay (ELISA, Ready-SET-Go!, eBioscience, San Diego, CA, USA), following the manufacturer's protocol. Light absorbance at 450 nm was read with the microplate reader VERSAmax (Molecular Devices LLC, San Jose, CA, USA) respectively.

### 9. Analysis of Tumor-Associated Macrophages (TAMs)

6132A tumor tissue was harvested at day 0, 6 and 20 after transfer of either H6- or αmL26-T cells. Single cell suspensions were prepared as described under (Tumor preparation) and incubated with DAF-FM (Life Technologies, Carlsbad, CA, USA) following manufacturer protocol for detection of NO expression. Fixation resistant dye fixable viability stain 780 (BD Bioscience, Franklin Lakes, NJ, USA) was used for detection of life/dead cells following manufacture protocol. Afterwards, cells were fixed and permeabilized using cytofix/cytoperm solution (BD Bioscience, Franklin Lakes, NJ, USA) following manufacture protocol followed by 1 μg Fc receptor block (anti-mouse 2.4G2). In the end, intracellular cytokine stain was performed together with CD11b and F4/80 and TAMs were analyzed by flow cytometry using NovoCyte Quanteon (Agilent, Santa Clara, CA, USA).

### 10. BrdU Injection and Cleaved Caspase 3

6132A-ECFP labeled cancer cells were used for these experiments. Mice were injected i.p. twice a day with 100 μL BrdU (Sigma-Aldrich, Burlington, MA, USA) at a concentration of 10 μg/μL for three consecutive days. Mice were sacrificed and tumor and spleen were taken out as described under tumor preparation and T cell cultures. BrdU stain was performed using the BD BrdU Flow kit (BD Bioscience, Franklin Lakes, NJ, USA) following manufacturer protocol. In addition, fixation resistant dye fixable viability stain 780 (BD Bioscience, Franklin Lakes, NJ, USA) was used for detection of life/dead cells. The rabbit antibody clone 9661 (Cell Signaling Technology, Danvers, MA, USA) was used for detection of cleaved caspase 3 and anti-rabbit IgG clone 79408 [R-Phycoerythrin (PE), Cell Signaling Technology, Danvers, MA, USA] was used for detection by flow cytometry. Furthermore, CD11b and F4/80 was used to detect TAMs and CD3, CD4 together with mL9-tetramer was used to detect TILs.

### 11. Tumor Infiltration and Peripheral Blood Analysis

Blood was taken by buccal bleeding between day 45 and 75 as indicated in the figure legends with a 5 mm animal lancet (Medipoint Inc, Mineola, NY, USA). Blood (100 μL) was collected in tubes containing 50 μL heparin (80 U/mL, Pfizer, New York, NY, USA). Red blood cells were lysed and remaining peripheral blood cells were stained with Sytox Blue (Helix NP Blue, Biolegend, San Diego, CA, USA) for life/dead cells and CD3, CD4 and Vb6 before analyzed by flow cytometry with the NovoCyte Quanteon (Agilent, Santa Clara, CA, USA). Same procedure as described under “Tumor preparation” was done to generate tumor cell suspensions that were analyzed for infiltration of T cells by flow cytometry. For determination of TILS, Sytox Blue, CD3, CD4 and mL9-tetramer was used.

### 12. Histology and Immunohistochemistry

Moribund mice were sacrificed by cervical dislocation and were subjected to a full necropsy. Tissue samples were fixed for 24 h in 10% buffered formalin (Sigma-Aldrich, Burlington, MA, USA) and then transferred to 70% ethanol. Tissue processing and immunohistochemistry stainings were performed by the Human Tissue Resource Center at the University of Chicago. Tissues were processed, paraffin embedded and 5 μm sections mounted on glass slides were subsequently stained with hematoxylin and eosin (H&E). Histopathological analysis was performed blinded and independently by two experienced pathologists. Microscopic images were captured using an Olympus BX43 microscope equipped with a ProgRes Speed XT core5 camera (Jenoptik) or a Leitz Laborlux D (W.Nuhsbaum, Inc., Mc Henry, IL, USA) microscope with a Retiga 2000R (QImaging) camera and Adobe Photoshop 2014 2.2 (San Jose, CA) to compose images. Serial sections were stained for CD3 with rabbit monoclonal antibody SP162 (abcam abl35372). The slides were stained using Leica Bond RX automated stainer. After dewax and rehydration, tissue section was heat treated for 20 min with antigen retrieval solution (Leica Biosystems, AR9961). Anti-CD3 antibody (1:100) was applied on tissue sections for 60 min incubation at room temperature and the antigen-antibody binding was detected with Bond Polymer Refine Detection HRP detection system (Leica Biosystems, DS9800) without post primary antibody amplification. The peroxidase reaction was developed using liquid diaminobenzidine brown substrate chromogen provided in the kit. Sections were counterstained with hematoxylin, dehydrated in alcohol, cleared in Xylene and mounted in Tissue-Tek Glas Mounting Medium (Sakura Finetek Japan Co, Ltd., Tokyo, Japan) for microscopic evaluation.

### 13. Longitudinal Confocal Imaging

The method was described before². Windows were implanted on the shaved back of C3H Rag^{-/-} mice. 6132A-cerulean cancer cells were injected at 3 different sites in between the fascia and dermis of the rear skin layer. Mice were treated 15 days after window implantation with anti-mRPL9 CD4⁺ TCR-transduced T cells. For longitudinal in vivo imaging, mice were anesthetized and positioned on a custom-made stage adaptor. The three screws that are used to hold the window frame also fixed the mouse onto the stage adaptor. A motorized microscope XY scanning stage and Leica LAS-AF software allowed recording individual 3-dimensional positions per field-of-view and returning to them later with high precision (stated accuracy ±3 μm; reproducibility <1.0 μm). Blood vessels were used as “landmarks” and could be located within 50 m on the same day and within 100 m on the next day. Data were acquired using a Leica SP5 II TCS tandem scanner two-photon spectral confocal microscope (long-working distance 20×/NA 0.45 and 4×/NA 0.16 dry lenses, Olympus). Tumor blood flow was visualized by retroorbital injection of 100 μL red blood cells labelled with DiD (1,1′-dioctadecyl-3,3,3′,3′-tetramethylindodicarbocyanine,4-chlorobenzenesulfonate salt, Thermo Fisher Scientific, Waltham, MA, USA). To determine the fraction of area occupied by vessels or cerulean fluorescent cancer cells, acquired images were analyzed using Fiji software (Laboratory for Optical and Computational Instrumentation; University of Wisconsin-Madison, WI).

### 14. Determination of MHC II Peptide Affinity

MHC purification and quantitative assays to measure the binding affinity of peptides to purified I E^{k} molecules were carried out as previously described (80,81).

15. Flow cytometry and antibodies

1 μg Fc receptor block (anti-mouse 2.4G2) was added to samples and cells were incubated with 50 μL phosphate buffer saline (PBS) containing 0.2 μg of indicated anti-mouse antibodies for 20 min at 4° C. Then samples were washed twice with PBS and acquired using NovoCyte Quanteon (Agilent, Santa Clara, CA, USA). Data analysis was performed using FlowJo software (TreeStar, Ashland, OR, USA). Antibodies: Arginase 1 [AlexF5, Allophycocyanin (APC), eFluor 450, eBioscience, Hatfield, GB], CD3⁺ [145-2C11, Fluorescein isothiocyanate (FITC), Peridinin chlorophyll protein-Cyanine5.5 (PerCp/Cy5.5)], CD4⁺ [GK1.5, Allophycocyanin (APC), Allophycocyanin-Cyanine7 (APC/Cy7), Brilliant Violet 421 (BV421), Fluorescein isothiocyanate (FITC)], CD11b⁺ [M1/70, Allophycocyanin (APC), Brilliant Violet 421 (BV421), R-Phycoerythrin (PE)], CD31⁺ [390, R-Phycoerythrin (PE)], CD146⁺ [ME-9F1, Allophycocyanin (APC)], F4/80⁺[BM8, Peridinin chlorophyll protein-Cyanine5.5 (PerCp/Cy5.5)], I-E^{k} [14-4-4S, Alexa Fluor 647 (AF647)], IL-10 [JES5-16E3, Brilliant Violet 421 (BV421)], IL-12 [C15.6, Allophycocyanin (APC)], TGF□ (TW7-20B9, R-Phycoerythrin (PE)], TNF [MP6-XT22, Allophycocyanin (APC), R-Phycoerythrin (PE)], TCR Vb2 [B20.6, R-Phycoerythrin (PE)], TCR Vb6 [RR4-7, R-Phycoerythrin (PE)]. If not indicated, antibodies were purchased from Biolegend (San Diego, CA, USA). Tetramers (I-E^{k}-mL9 and I-E^{k}-CLIP) were provided by the NIH Tetramer Core Facility. Samples were stained with 1.4 μg/mL tetramer for 1 h at 4° C. in Roswell Park Memorial Institute medium (RPMI 1640, Corning, Corning, NY, USA) containing 10% FBS (Gemini, Sacramento, CA, USA). For life/dead distinction, Sytox Blue (Helix NP Blue, Biolegend, San Diego, CA, USA), Fixation resistant dye fixable viability stain 510 or 780 (BD Bioscience, Franklin Lakes, NJ, USA) were used.

### 16. Whole-Exome and RNA Sequencing of Cancer Cells

Both genomic DNA and total RNA were extracted from the 6132A and 6139B cell lines, using AllPrep DNA/RNA mini kit (Qiagen, Venlo, The Netherlands). For whole-exome sequencing, 3 μg of genomic DNA was subjected to library construction, using SureSeletXT Mouse All Exon VI (Agilent Technologies, Santa Clara, CA, USA). RNAseq libraries were prepared from 1 μg of total RNA using TruSeq Stranded Total RNA Library Prep kit (Illumina, San Diego, CA, USA). The prepared whole-exome and RNAseq libraries were quantified by 2200 Tape Station (Agilent Technologies, Santa Clara, CA, USA), and then sequenced by 150 bp paired-end reads on NextSeq 500 Sequencer (Illumina, San Diego, CA, USA).

### 17. Full Thickness Skin Grafting

As described earlier (82), donor skin (BALB/cAnN) along the dorsal surface was obtained and around 2 cm² were applied to the dorsal thoracic wall of recipient mice (B6 Rag^{-/-} or C3H Rag^{-/-}). Bandages were removed on day 7 and on day 12 cancer cells were injected s.c. on the other flank of the same mouse. For adoptive T cell transfer, spleen cells of TCR75-transgenic mice that recognize the K^{d} 15mer QEGPEYWEEQTQRAK presented on MHC class II I-A^{b} and reject BALB/c skin grafts within 14 days after T cell transfer (32) were transduced with the anti-mRPL9 CD4⁺ TCR. Grafts were monitored daily until rejection (defined as loss of at least 80% of grafted tissue) or the end point of the experiment.

### 18. TCR Sequencing Analysis

The raw sequencing data were processed using the 10× Genomics Cell Ranger Software (v6.0.0) with the command cellranger multi, the provided config csv files contains the information of mm10 reference genome, vdj GRCm38 reference and TotalSeq CD45 surface markers. The output from cellranger multi contains both gene expression metric and TCR diversity metric which includes clonotype frequency and barcode information. The sample feature be matrix of each sample was then converted to Seurat v4.0 (83) to build a Seurat object containing both RNA assay and ADT (antibody-derived tags) assay. T cell clonotypes' information was then added to the Seurat object using an in-house R script. Low-quality cells were removed (200<nFeature_RNA <5000, percent of mitochondrial genes <10). The spleen and tumor samples were then merged and subjected to log normalization, data scaling with the top 5000 most variable genes and PCA analysis. Batch effects were corrected with Harmony (84) using “RunHarmony” function in Seurat.

### 19. Statistics

All statistical analyses were performed using GraphPad Prism software (GraphPad, San Diego, CA, USA). Data points indicate either means of biological duplicates of a representative experiment or are experimental replicates summarized as mean±standard deviation. The method used to present the statistical significance of the data is always indicated in the figure legend.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The references described herein, to the extent that they provide exemplary procedural or other details supplementary to those set forth herein, are specifically incorporated herein by reference.
1 Tabin, C. J. et al. Mechanism of activation of a human oncogene. Nature 300, 143-149 (1982).
2 Vogelstein, B. et al. Cancer genome landscapes. Science 339, 1546-1558, doi:10.1126/science.1235122 (2013).
3 Reddy, E. P., Reynolds, R. K., Santos, E. & Barbacid, M. A point mutation is responsible for the acquisition of transforming properties by the T24 human bladder carcinoma oncogene. Nature 300, 149-152 (1982).
4 Monach, P. A., Meredith, S. C., Siegel, C. T. & Schreiber, H. A unique tumor antigen produced by a single amino acid substitution. Immunity 2, 45-59 (1995).
5 van Rooij, N. et al. Tumor exome analysis reveals neoantigen-specific T-cell reactivity in an ipilimumab-responsive melanoma. J Clin Oncol 31, e439-442, doi:10.1200/JCO.2012.47.7521 (2013).
6 Robbins, P. F. et al. Mining exomic sequencing data to identify mutated antigens recognized by adoptively transferred tumor-reactive T cells. Nat Med 19, 747-752, doi:10.1038/nm.3161 (2013).
7 Tran, E., Robbins, P. F. & Rosenberg, S. A. ‘Final common pathway’ of human cancer immunotherapy: targeting random somatic mutations. Nat Immunol 18, 255-262, doi:10.1038/ni.3682 (2017).
8 Haslam, A. & Prasad, V. Estimation of the Percentage of US Patients With Cancer Who Are Eligible for and Respond to Checkpoint Inhibitor Immunotherapy Drugs. JAMA Netw Open 2, e192535, doi:10.1001/jamanetworkopen.2019.2535 (2019).
9 Rosenberg, S. A. & Restifo, N. P. Adoptive cell transfer as personalized immunotherapy for human cancer. Science 348, 62-68, doi:10.1126/science.aaa4967 (2015). Schietinger, A., Delrow, J. J., Basom, R. S., Blattman, J. N. & Greenberg, P. D. Rescued tolerant CD8 T cells are preprogrammed to reestablish the tolerant state. Science 335, 723-727, doi:10.1126/science.1214277 (2012).
11 Philip, M. et al. Chromatin states define tumour-specific T cell dysfunction and reprogramming. Nature 545, 452-456, doi:10.1038/nature22367 (2017).
12 Kim, S. P. et al. Adoptive Cellular Therapy with Autologous Tumor-Infiltrating Lymphocytes and T-cell Receptor-Engineered T Cells Targeting Common p53 Neoantigens in Human Solid Tumors. Cancer Immunol Res, OF1-OF15, doi:10.1158/2326-6066.CIR-22-0040 (2022).
13 Leidner, R. et al. Neoantigen T-Cell Receptor Gene Therapy in Pancreatic Cancer. N Engl J Med 386, 2112-2119, doi:10.1056/NEJMoa2119662 (2022).
14 Leisegang, M. et al. Eradication of Large Solid Tumors by Gene Therapy with a T-Cell Receptor Targeting a Single Cancer-Specific Point Mutation. Clin Cancer Res 22, 2734-2743, doi:10.1158/1078-0432.CCR-15-2361 (2016).
15 Dersh, D., Holly, J. & Yewdell, J. W. A few good peptides: MHC class I-based cancer immunosurveillance and immunoevasion. Nat Rev Immunol 21, 116-128, doi:10.1038/s41577-020-0390-6 (2021).
16 Philip, M. & Schietinger, A. CD8(+) T cell differentiation and dysfunction in cancer. Nat Rev Immunol 22, 209-223, doi:10.1038/s41577-021-00574-3 (2022).
17 Oliveira, G. et al. Phenotype, specificity and avidity of antitumour CD8(+) T cells in melanoma. Nature 596, 119-125, doi:10.1038/s41586-021-03704-y (2021).
18 Tran, E. et al. Cancer immunotherapy based on mutation-specific CD4+ T cells in a patient with epithelial cancer. Science 344, 641-645, doi:344/6184/641 [pii]10.1126/science.1251102 (2014).
19 Veatch, J. R. et al. Tumor-infiltrating BRAFV600E-specific CD4+ T cells correlated with complete clinical response in melanoma. J Clin Invest 128, 1563-1568, doi:10.1172/JC198689 (2018).
20 Zacharakis, N. et al. Immune recognition of somatic mutations leading to complete durable regression in metastatic breast cancer. Nat Med 24, 724-730, doi:10.1038/s41591-018-0040-8 (2018).
21 Ahrends, T. et al. CD4(+) T Cell Help Confers a Cytotoxic T Cell Effector Program Including Coinhibitory Receptor Downregulation and Increased Tissue Invasiveness. Immunity 47, 848-861 e845, doi:10.1016/j.immuni.2017.10.009 (2017).
22 Schietinger, A., Philip, M., Liu, R. B., Schreiber, K. & Schreiber, H. Bystander killing of cancer requires the cooperation of CD4(+) and CD8(+) T cells during the effector phase. J Exp Med 207, 2469-2477, doi:10.1084/jem.20092450 (2010).
23 Bos, R. & Sherman, L. A. CD4+ T-cell help in the tumor milieu is required for recruitment and cytolytic function of CD8+ T lymphocytes. Cancer Res 70, 8368-8377, doi:10.1158/0008-5472.CAN-10-1322 (2010).
24 Van Waes, C., Urban, J. L., Rothstein, J. L., Ward, P. L. & Schreiber, H. Highly malignant tumor variants retain tumor-specific antigens recognized by T helper cells. J. Exp. Med. 164, 1547-1565 (1986).
25 Ward, P. L., Koeppen, H., Hurteau, T. & Schreiber, H. Tumor antigens defined by cloned immunological probes are highly polymorphic and are not detected on autologous normal cells. J Exp Med 170, 217-232 (1989).
26 Beck-Engeser, G. B. et al. Point mutation in essential genes with loss or mutation of the second allele: relevance to the retention of tumor-specific antigens. J Exp Med 194, 285-300 (2001).
27 Schietinger, A. et al. Longitudinal confocal microscopy imaging of solid tumor destruction following adoptive T cell transfer. Oncoimmunology 2, e26677, doi:10.4161/onci.26677 (2013).
28 Kammertoens, T. et al. Tumour ischaemia by interferon-gamma resembles physiological blood vessel regression. Nature 545, 98-102, doi:10.1038/nature22311 (2017).
29 Tang, H. L. et al. Cell survival, DNA damage, and oncogenic transformation after a transient and reversible apoptotic response. Mol Biol Cell 23, 2240-2252, doi:10.1091/mbc.E11-11-0926 (2012).

Liu, X. et al. Caspase-3 promotes genetic instability and carcinogenesis. Mol Cell 58, 284-296, doi:10.1016/j.molcel.2015.03.003 (2015).
31 Mumberg, D. et al. CD4(+) T cells eliminate MHC class II-negative cancer cells in vivo by indirect effects of IFN-gamma. Proc Natl Acad Sci USA 96, 8633-8638 (1999).
32 Honjo, K., Xu, X. & Bucy, R. P. CD4+ T-cell receptor transgenic T cells alone can reject vascularized heart transplants through the indirect pathway of alloantigen recognition. Transplantation 77, 452-455, doi:10.1097/01.TP.0000112937.12491.42 (2004).
33 Dubey, P. et al. The immunodominant antigen of an ultraviolet-induced regressor tumor is generated by a somatic point mutation in the DEAD box helicase p68. J Exp Med 185, 695-705 (1997).
34 Matsushita, H. et al. Cancer exome analysis reveals a T-cell-dependent mechanism of cancer immunoediting. Nature 482, 400-404, doi:10.1038/nature10755 (2012).
35 Ward, P. L., Koeppen, H. K., Hurteau, T., Rowley, D. A. & Schreiber, H. Major histocompatibility complex class I and unique antigen expression by murine tumors that escaped from CD8+ T-cell-dependent surveillance. Cancer Res 50, 3851-3858 (1990).
36 Wolf, S. P., Wen, F. T. & Schreiber, H. Criteria to make animal studies more relevant to treating human cancer. Curr Opin Immunol 74, 25-31, doi:10.1016/j.coi.2021.09.002 (2021).
37 Wortzel, R. D., Philipps, C. & Schreiber, H. Multiple tumour-specific antigens expressed on a single tumour cell. Nature 304, 165-167 (1983).
38 Schreiber, K. et al. Impact of TCR Diversity on the Development of Transplanted or Chemically Induced Tumors. Cancer Immunol Res 8, 192-202, doi:10.1158/2326-6066.CIR-19-0567 (2020).
39 Venturi, V. et al. Sharing of T cell receptors in antigen-specific responses is driven by convergent recombination. Proc Natl Acad Sci USA 103, 18691-18696, doi:10.1073/pnas.0608907103 (2006).
40 Quigley, M. F. et al. Convergent recombination shapes the clonotypic landscape of the naive T-cell repertoire. Proc Natl Acad Sci USA 107, 19414-19419, doi:10.1073/pnas.1010586107 (2010).
41 Hunder, N. N. et al. Treatment of metastatic melanoma with autologous CD4+ T cells against NY-ESO-1. N Engl J Med 358, 2698-2703, doi:10.1056/NEJMoa0800251 (2008).
42 Muranski, P. et al. Tumor-specific Th17-polarized cells eradicate large established melanoma. Blood 112, 362-373, doi:blood-2007-11-120998 [pii]10.1182/blood-2007-11-120998 (2008).
43 Quezada, S. A. et al. Tumor-reactive CD4(+) T cells develop cytotoxic activity and eradicate large established melanoma after transfer into lymphopenic hosts. J Exp Med 207, 637-650, doi:10.1084/jem.20091918 (2010).
44 Greenberg, P. D., Kern, D. E. & Cheever, M. A. Therapy of disseminated murine leukemia with cyclophosphamide and immune Lyt-1+,2-T cells. Tumor eradication does not require participation of cytotoxic T cells. J Exp Med 161, 1122-1134, doi:10.1084/jem.161.5.1122 (1985).
45 Kreiter, S. et al. Mutant MHC class II epitopes drive therapeutic immune responses to cancer. Nature 520, 692-696, doi:10.1038/naturel4426 (2015).
46 Linnemann, C. et al. High-throughput epitope discovery reveals frequent recognition of neo-antigens by CD4+ T cells in human melanoma. Nat Med 21, 81-85, doi:10.1038/nm.3773 (2015).
47 Johnson, D. B. et al. Melanoma-specific MHC-II expression represents a tumor-autonomous phenotype and predicts response to anti-PD-1/PD-L1 therapy. Nature Communications, doi:10.1038/ncomms10582 (2016).
48 Abelin, J. G. et al. Defining HLA-II Ligand Processing and Binding Rules with Mass Spectrometry Enhances Cancer Epitope Prediction. Immunity 51, 766-779 e717, doi:10.1016/j.immuni.2019.08.012 (2019).
49 Tveita, A. A. et al. Indirect CD4+ T-cell-mediated elimination of MHC II(NEG) tumor cells is spatially restricted and fails to prevent escape of antigen-negative cells. Eur J Immunol 44, 2625-2637, doi:10.1002/eji.201444659 (2014).
50 Baik, I. H. et al. Knockdown of RPL9 expression inhibits colorectal carcinoma growth via the inactivation of Id-1/NF-kappaB signaling axis. Int J Oncol 49, 1953-1962, doi:10.3892/ijo.2016.3688 (2016).
51 Takagi, M., Absalon, M. J., McLure, K. G. & Kastan, M. B. Regulation of p53 translation and induction after DNA damage by ribosomal protein L26 and nucleolin. Cell 123, 49-63, doi:10.1016/j.cell.2005.07.034 (2005).
52 Nichols, C. A. et al. Loss of heterozygosity of essential genes represents a widespread class of potential cancer vulnerabilities. Nat Commun 11, 2517, doi:10.1038/s41467-020-16399-y (2020).
53 Hwang, M. S. et al. Targeting loss of heterozygosity for cancer-specific immunotherapy. Proc Natl Acad Sci USA 118, doi:10.1073/pnas.2022410118 (2021).
54 Zhang, X. & Sjoblom, T. Targeting Loss of Heterozygosity: A Novel Paradigm for Cancer Therapy. Pharmaceuticals (Basel) 14, doi:10.3390/ph14010057 (2021).
55 Lowery, F. J. et al. Molecular signatures of antitumor neoantigen-reactive T cells from metastatic human cancers. Science 375, 877-884, doi:10.1126/science.abl5447 (2022).
56 Oliveira, G. et al. Landscape of helper and regulatory antitumour CD4(+) T cells in melanoma. Nature 605, 532-538, doi:10.1038/s41586-022-04682-5 (2022).
57 Yost, K. E. et al. Clonal replacement of tumor-specific T cells following PD-1 blockade. Nat Med 25, 1251-1259, doi:10.1038/s41591-019-0522-3 (2019).
58 Hayflick, L. The Limited in Vitro Lifetime of Human Diploid Cell Strains. Exp Cell Res 37, 614-636, doi:10.1016/0014-4827(65)90211-9 (1965).
59 Joshi, N. S. et al. Increased numbers of preexisting memory CD8 T cells and decreased T-bet expression can restrain terminal differentiation of secondary effector and memory CD8 T cells. J Immunol 187, 4068-4076, doi:10.4049/jimmunol.1002145 (2011).
60 Imamichi, H. et al. Lifespan of effector memory CD4+ T cells determined by replication-incompetent integrated HIV-1 provirus. AIDS 28, 1091-1099, doi:10.1097/QAD.0000000000000223 (2014).
61 Binnewies, M. et al. Understanding the tumor immune microenvironment (TIME) for effective therapy. Nat Med 24, 541-550, doi:10.1038/s41591-018-0014-x (2018).
62 Seung, L. P., Rowley, D. A., Dubey, P. & Schreiber, H. Synergy between T-cell immunity and inhibition of paracrine stimulation causes tumor rejection. Proc Natl Acad Sci USA 92, 6254-6258, doi:10.1073/pnas.92.14.6254 (1995).
63 Kammertoens, T. et al. Tumour ischaemia by interferon-gamma resembles physiological blood vessel regression. Nature 545, 98-102, doi:10.1038/nature22311 (2017).
64 Braumuller, H. et al. T-helper-1-cell cytokines drive cancer into senescence. Nature 494, 361-365, doi:naturel1824 [pii]10.1038/nature11824 (2013).
65 Fauskanger, M., Haabeth, O. A. W., Skjeldal, F. M., Bogen, B. & Tveita, A. A. Tumor Killing by CD4(+) T Cells Is Mediated via Induction of Inducible Nitric Oxide Synthase-Dependent Macrophage Cytotoxicity. Front Immunol 9, 1684, doi:10.3389/fimmu.2018.01684 (2018).
66 Bogen, B., Fauskanger, M., Haabeth, O. A. & Tveita, A. CD4(+) T cells indirectly kill tumor cells via induction of cytotoxic macrophages in mouse models. Cancer Immunol Immunother 68, 1865-1873, doi:10.1007/s00262-019-02374-0 (2019).
67 Zhang, B. et al. Induced sensitization of tumor stroma leads to eradication of established cancer by T cells. J Exp Med 204, 49-55 (2007).
68 Zhang, B. et al. Equilibrium between host and cancer caused by effector T cells killing tumor stroma. Cancer Res. 68, 1563-1571 (2008).
69 Stuehr, D. J. & Nathan, C. F. Nitric oxide. A macrophage product responsible for cytostasis and respiratory inhibition in tumor target cells. J Exp Med 169, 1543-1555, doi:10.1084/jem.169.5.1543 (1989).
70 Kim, Y. M., Bombeck, C. A. & Billiar, T. R. Nitric oxide as a bifunctional regulator of apoptosis. Circ Res 84, 253-256, doi:10.1161/01.res.84.3.253 (1999).
71 Diehl, M. I., Wolf, S. P., Bindokas, V. P. & Schreiber, H. Automated cell cluster analysis provides insight into multi-cell-type interactions between immune cells and their targets. Exp Cell Res, 112014, doi:10.1016/j.yexcr.2020.112014 (2020).
72 Zhang, Z. et al. RNF2 ablation reprograms the tumor-immune microenvironment and stimulates durable NK and CD4(+) T-cell-dependent antitumor immunity. Nat Cancer 2, 1018-1038, doi:10.1038/s43018-021-00263-z (2021).
73 Doench, J. G. et al. Optimized sgRNA design to maximize activity and minimize off-target effects of CRISPR-Cas9. Nat Biotechnol 34, 184-191, doi:10.1038/nbt.3437 (2016).
74 Ran, F. A. et al. Genome engineering using the CRISPR-Cas9 system. Nat Protoc 8, 2281-2308, doi:10.1038/nprot.2013.143 (2013).
75 Morita, S., Kojima, T. & Kitamura, T. Plat-E: an efficient and stable system for transient packaging of retroviruses. Gene Ther 7, 1063-1066, doi:10.1038/sj.gt.3301206 (2000).
76 Engels, B., Chervin, A. S., Sant, A. J., Kranz, D. M. & Schreiber, H. Long-term persistence of CD4(+) but rapid disappearance of CD8(+) T cells expressing an MHC class I-restricted TCR of nanomolar affinity. Mol Ther 20, 652-660, doi:10.1038/mt.2011.286 (2012).
77 Engels, B. et al. Relapse or eradication of cancer is predicted by peptide-major histocompatibility complex affinity. Cancer Cell 23, 516-526, doi:10.1016/j.ccr.2013.03.018 (2013).
78 Spiotto, M. T., Rowley, D. A. & Schreiber, H. Bystander elimination of antigen loss variants in established tumors. Nat Med 10, 294-298, doi:10.1038/nm999 (2004).
79 Philip, M., Schietinger, A. & Schreiber, H. Ribosomal versus non-ribosomal cellular antigens: factors determining efficiency of indirect presentation to CD4+ T cells. Immunology 130, 494-503, doi:10.1111/j.1365-2567.2010.03258.x (2010).
80 Sidney, J. et al. Measurement of MHC/peptide interactions by gel filtration or monoclonal antibody capture. Curr Protoc Immunol Chapter 18, Unit 18 13, doi:10.1002/0471142735.im1803s100 (2013).
81 Sidney, J. et al. Measurement of MHC/peptide interactions by gel filtration. Curr Protoc Immunol Chapter 18, Unit 18 13, doi:10.1002/0471142735.im1803s31 (2001).
82 Wick, M. et al. Antigenic cancer cells grow progressively in immune hosts without evidence for T cell exhaustion or systemic anergy. J Exp Med 186, 229-238, doi:10.1084/jem.186.2.229 (1997).
83 Stuart, T. et al. Comprehensive Integration of Single-Cell Data. Cell 177, 1888-1902 e1821, doi:10.1016/j.cell.2019.05.031 (2019).
84 Korsunsky, I. et al. Fast, sensitive and accurate integration of single-cell data with Harmony. Nat Methods 16, 1289-1296, doi:10.1038/s41592-019-0619-0 (2019).

1. A method for isolating and/or expanding neoantigen-specific immune cells from a subject comprising
contacting a starting population of in vitro immune effector cells obtained from the subject with either i) a population of B cells comprising an expression vector encoding 2-15 minigenes linked to a promoter and wherein each minigene encodes a different neoantigen; or ii) cell lysate obtained from a population of B cells comprising an expression vector encoding 2-15 minigenes linked to a promoter and wherein each minigene encodes a different neoantigen.

2. A B cell comprising an expression vector that encodes at least one minigene linked to a promoter and wherein the minigene encodes a neoantigen.

3. A B cell comprising an expression vector that encodes at least one minigene linked to a promoter and wherein the minigene encodes a wild-type polypeptide that corresponds to a neoantigen.

4. The B cell of claim 2 or 3, wherein the cell is isolated from a subject having cancer.

5. The B cell of any one of claims 2-4, wherein the neoantigen is a neoantigen expressed in a cancer cell from the subject.

6. The B cell of claim 5, wherein the neoantigen comprises a non-synonymous single nucleotide variant (nsSNV) or frameshift mutation.

7. The B cell of claim 6, wherein the nsSNV is in the center position of the minigene or in one of the center positions of the minigene.

8. The B cell of claim 3 or 4, wherein the wild-type polypeptide is the wild-type polypeptide that corresponds to a neoantigen comprising a nsSNV or frameshift mutation.

9. The B cell of claim 8, wherein the nucleotide corresponding to the nsSNV is in the center position of the minigene or in one of the center positions of the minigene.

10. The B cell of any one of claims 2-9, wherein the B cell comprises an expression vector that encodes a non-immunogenic heterologous cell marker.

11. The B cell of claim 10, wherein the non-immunogenic heterologous cell marker is CD11c.

12. The B cell of any one of claims 2-11, wherein the vector encodes at least 2 minigenes linked to a promoter.

13. The B cell of claim 12, wherein the vector encodes 2-15 minigenes linked to a promoter.

14. The B cell of claim 13, wherein each minigene encodes a different neoantigen.

15. The B cell of claim 13, wherein each minigene encodes a different wild-type polypeptide that corresponds to a neoantigen.

16. The B cell of any one of claims 13-15, wherein the vector encodes one or more proteasomal cleavage sites between each minigene.

17. The B cell of claim 16, wherein the proteasomal cleavage site comprises the amino acid sequence: AAY.

18. The B cell of any one of claims 10-17, wherein the cell marker and the minigene(s) are encoded on the same vector.

19. The B cell of claim 18, wherein the cell marker and minigene(s) are expressed from the same promoter.

20. The B cell of claim 18 or 19, wherein the vector encodes for a self-cleaving peptide between the minigene(s) and the cell marker.

21. The B cell of claim 20, wherein the self-cleaving peptide is a 2A-element.

22. The B cell of any one of claims 2-21, wherein each minigene encodes for a peptide having 10-30 amino acids in length.

23. The B cell of claim 22, wherein each minigene encodes for a peptide having 25 amino acids in length.

24. The B cell of any one of claims 2-23, wherein the expression vector is integrated into the genome of the B cell.

25. The B cell of any one of claims 2-24, wherein the B cell is CD19+.

26. A population of cells comprising at least two or more B cells according to any one of claims 2-25, wherein each B cell comprises the same vector.

27. A method for generating a population of conditionally immortal B cells comprising:
isolating B cells from a patient;
transferring a vector comprising at least one minigene linked to a promoter into the cells; wherein the minigene encodes a neoantigen; and
contacting the cells with a composition comprising one or both of CD40 ligand (CD40L) and TL-4, thereby generating a population of conditionally immortal B cells.

28. A method for generating a population of conditionally immortal B cells comprising:
isolating B cells from a subject;
transferring a vector comprising at least one minigene linked to a promoter into the cells; wherein the minigene encodes a wild-type polypeptide that corresponds to a neoantigen; and
contacting the cells with a composition comprising one or both of CD40 ligand (CD40L) and TL-4, thereby generating a population of conditionally immortal B cells.

29. The method of claim 27 or 28, wherein the cells are isolated from a subject having cancer.

30. The method of any one of claims 27-29, wherein the neoantigen is a neoantigen expressed in a cancer cell from the subject.

31. The method of claim 30, wherein the neoantigen comprises a non-synonymous single nucleotide variant (nsSNV) or frameshift mutation.

32. The method of claim 31, wherein the nsSNV is in the center position of the minigene or in one of the center positions of the minigene.

33. The method of claim 28 or 29, wherein the wild-type polypeptide is the wild-type polypeptide that corresponds to a neoantigen comprising a nsSNV or frameshift mutation.

34. The method of claim 33, wherein the nucleotide corresponding to the nsSNV is in the center position of the minigene or in one of the center positions of the minigene.

35. The method of any one of claims 27-34, wherein the B cells comprise an expression vector that encodes a non-immunogenic heterologous cell marker.

36. The method of claim 35, wherein the non-immunogenic heterologous cell marker is CD11c.

37. The method of any one of claims 27-36, wherein the vector encodes at least 2 minigenes linked to a promoter.

38. The method of claim 37, wherein the vector encodes 2-15 minigenes linked to a promoter.

39. The method of claim 38, wherein each minigene encodes a different neoantigen.

40. The method of claim 38, wherein each minigene encodes a different wild-type polypeptide that corresponds to a neoantigen.

41. The method of any one of claims 38-40, wherein the vector encodes one or more proteasomal cleavage sites between each minigene.

42. The method of claim 41, wherein the proteasomal cleavage site comprises the amino acid sequence: AAY.

43. The method of any one of claims 35-42, wherein the cell marker and the minigene(s) are encoded on the same vector.

44. The method of claim 43, wherein the cell marker and minigene(s) are expressed from the same promoter.

45. The method of claim 43 or 44, wherein the vector encodes for a self-cleaving peptide between the minigene(s) and the cell marker.

46. The method of claim 45, wherein the self-cleaving peptide is a 2A-element.

47. The method of any one of claims 27-46, wherein each minigene encodes for a peptide having s 10-30 amino acids in length.

48. The method of claim 47, wherein each minigene encodes for a peptide having 25 amino acids in length.

49. The method of any one of claims 27-48, wherein the expression vector is integrated into the genome of the B cells.

50. The method of any one of claims 27-49, wherein the B cells are CD19+.

51. The method of any one of claims 27-50, wherein the method further comprises enriching the population of cells for the heterologous cell marker and/or for CD19.

52. The method of claim 51, wherein enriching the population of cells for the heterologous cell marker and/or for CD19 comprising sorting the cells based on expression of the heterologous cell marker and/or CD19.

53. The method of any one of claims 27-52, wherein the CD40L comprises irradiated CD40L-positive cells.

54. The method of any one of claims 27-52, wherein the composition comprises 1-2 ng/mL or 10-30 U/mL IL-4.

55. A population of cells created by the method of any one of claims 27-54.

56. A pool of 2 or more populations of B cells according to claim 26 or 55, wherein each population comprises a different vector.

57. The pool of cells of claim 56, wherein the pool of cells comprises 2-6 populations.

58. A method for preparing a cell lysate comprising freezing and thawing the population of B cells of claim 55 or the pool of B cells of claim 56 or 57.

59. A cell lysate produced by the method of claim 58.

60. A composition comprising the population of B cells of claim 55, the pool of B cells of claim 56 or 57, or the cell lysate of claim 59.

61. The composition of claim 60, wherein the B cells are conditionally immortal.

62. The composition of claim 60 or 61, wherein the composition comprises one or both of CD40 ligand (CD40L) and IL4.

63. The composition of claim 62, wherein the composition comprises irradiated CD40L-positive cells.

64. The composition of claim 62 or 63, wherein the composition comprises 1-2 ng/mL or 10-30 U/mL IL4.

65. A method for isolating and/or expanding neoantigen-specific immune cells from a subject comprising contacting a starting population of in vitro immune effector cells from the with the population of cells of claim 55, pool of cells of claim 56 or 57, cell lysate of claim 59 or composition of any one of claims 60-64, thereby generating neoantigen-specific immune cells.

66. The method of claim 65, wherein the neoantigen-specific immune cells comprise T cells.

67. The method of claim 65 or 66, wherein the cells from the subject are contacted in vitro with the cell lysate of claim 59 or a cell lysate of the population of cells of claim 55, pool of cells of claim 56 or 57, or composition of any one of claims 60-64.

68. The method of claim 67, wherein the method further comprises contacting the cells from the subject with antigen presenting cells (APCs), artificial antigen presenting cells (aAPCs), or an artificial antigen presenting surface (aAPSs).

69. The method of claim 68, wherein the APCs are dendritic cells.

70. The method of claim 69, wherein the APCs are peripheral blood derived dendritic cells.

71. The method of claim 68, wherein the APCs are conditionally immortalized B cells.

72. The method of any one of claims 65-71, wherein the cells from the subject are from a biopsy or tissue sample from the subject, or a fraction thereof.

73. The method of claim 72, wherein the biopsy or tissue sample comprises cancerous cells.

74. The method of claim 73, wherein the biopsy or tissue sample comprises tumor infiltrating lymphocytes (TILs).

75. The method of claim 65, wherein the cells from the subject are from a peripheral blood sample from the subject.

76. The method of any one of claims 65-75, wherein the method further comprises generating a clonal population of neoantigen-specific immune cells by limiting or serial dilution followed by expansion of individual clones by a rapid expansion protocol.

77. The method of any one of claims 65-76, wherein the method further comprises analyzing stimulation of the cells from the subject after they have been contacted with the population of cells, pool of cells, or composition.

78. The method of claim 77, wherein analyzing stimulation of the cells comprises evaluating the interferon gamma (IFNg) production after (ii).

79. The method of any one of claims 76-78, wherein the method further comprises cloning of a T cell receptor (TCR) from neoantigen-specific immune effector cells.

80. The method of claim 79, wherein cloning of the TCR is cloning of a TCR alpha and a beta chain.

81. The method of claim 79 or claim 80, wherein the TCR is identified using a 5′-Rapid amplification of cDNA ends (RACE) method or TCR-sequencing from single T cells.

82. The method of claim 81, wherein the cloned TCR is subcloned into an expression vector.

83. The method of claim 82, wherein the expression vector is a retroviral, lentiviral, or CRISPR/Cas9 vector.

84. The method of claim 82 or 83, where the expression vector is transferred to the host cell to generate an engineered cell that expresses the TCR.

85. The method of claim 84, wherein the expression vector is transferred to the host cell by transduction, transfection, or electroporation.

86. The method of claim 84 or 85, wherein the host cell is an immune cell.

87. The method of claim 86, wherein the immune cell is a T cell.

88. The method of claim 87, wherein the T cell is a CD8⁺ T cell, CD4⁺ T cell, or T6 T cell.

89. The method of any one of claims 65-88, wherein the subject has cancer.

90. The method of any one of claims 65-89, wherein the population of cells or pool of cells are autologous cells or wherein the composition comprises autologous cells.

91. The method of any one of claims 84-90, wherein a population of CD8-positive and neoantigen MHC tetramer-positive engineered immune cells are purified from the host cells.

92. The method of any one of claims 65-91, wherein the method further comprises contacting the neoantigen-specific immune cells or engineered immune cells with the population of cells of claim 55, pool of cells of claim 56 or 57, cell lysate of claim 59 or composition of any one of claims 60-64, wherein the minigene encodes a wild-type polypeptide that corresponds to a neoantigen.

93. The method of any one of claims 65-92, wherein the method further comprises contacting the neoantigen-specific immune cells or engineered immune cells with tumor cells or lysates thereof.

94. The method of claim 92 or 93, wherein the method further comprises analyzing immune cell stimulation of the population of immune cells after they have been contacted with the population of cells, pool of cells, composition, tumor cells, or lysates thereof.

95. The method of claim 94, wherein analyzing immune cell stimulation comprises evaluating the interferon gamma (IFNg) secretion of the cells.

96. A neoantigen-specific immune cell or TCR produced according to any one of the methods of claims 84-94.

97. The neoantigen-specific T cell of claim 96, wherein the engineered immune cells is contacted with cells or cell lysate comprising a minigene that encodes a wild-type polypeptide that corresponds to a neoantigen and wherein the T cells were determined to be unstimulated.

98. The neoantigen-specific immune cell of claim 96 or 97, wherein the engineered immune cells are contacted with tumor cells or lysates thereof and wherein T cells were determined to be stimulated.

99. The neoantigen-specific immune cell of claim 97 or 98, wherein the cell is a T cell.

100. A method for identifying cross-reactive engineered T cells comprising: contacting a population of subject-derived T cells from a subject with the B cells of any one of claims 3-25, or a cell lysate thereof; and
evaluating the stimulation of the T cells after contact with the B cells.

101. The method of claim 100, wherein the patient-derived T cells and the B cells are derived from the same subject.

102. The method of claim 100 or 101, wherein evaluating the stimulation of the T cells comprises evaluating the level of IFNγ production.

103. The method of any one of claims 100-102, wherein the T cells are determined to be cross-reactive when the T cells are evaluated as being stimulated after contact with the B cells.

104. The method of any one of claims 100-102, wherein the T cells are determined to be non-cross-reactive when the T cells are evaluated as being unstimulated after contact with the B cells.

105. The method of any one of claims 100-104, wherein the population of subject-derived T cells comprises a clonal or polyclonal population.

106. The method of any one of claims 100-104, wherein the population of subject-derived T cells are an expanded population of TILs or peripheral blood T cells derived from the subject.

107. A method for treating or vaccinating a subject for cancer comprising administering the population of cells of claim 55, pool of cells of claim 56 or 57, cell lysate of claim 59 or composition of any one of claims 60-64.

108. The method of claim 107, wherein the administered cells are autologous.

109. The method of claim 107 or 108, wherein the administered cells are proliferation-incompetent.

110. The method of any one of claims 107-109, wherein the administered cells are irradiated cells.

111. The method of any one of claims 107-110, wherein the subject has been diagnosed with cancer.

112. The method of any one of claims 107-111, wherein the subject is a human.

113. The method of any one of claims 107-112, further comprising administering at least a second additional therapy.

114. The method of claim 113, wherein the second therapy is an anti-cancer agent.

115. The method of any one of claims 107-114, wherein treating comprises one or more of reducing tumor size; increasing the overall survival rate; reducing the risk of recurrence of the cancer; reducing the risk of progression; and/or increasing the chance of progression-free survival, relapse-free survival, and/or recurrence-free survival.

116. A method for treating or vaccinating a subject for cancer comprising: administering autologous engineered T cells to the subject, wherein the engineered T cells comprise:
(i) CD8+ T cells that are stimulated in response to at least one MHC class I restricted neoantigen; and
(ii) CD4+ T cells that are stimulated in response to at least one MHC class II restricted neoantigen.

117. The method of claim 116, wherein the CD8+ T cells comprise engineered T cells that are stimulated in response to one MHC class I restricted neoantigen; and/or the CD4+ T cells comprise engineered T cells that are stimulated in response to one MHC class II restricted neoantigen.

118. The method of claim 116 or 117, wherein the subject has been diagnosed with cancer.

119. The method of any one of claims 116-118, wherein the subject is a human.

120. The method of any one of claims 116-119, further comprising administering at least a second therapeutic agent.

121. The method of claim 120, wherein the second therapeutic agent is an anti-cancer agent.

122. The method of any one of claims 116-121, wherein treating comprises one or more of reducing tumor size; increasing the overall survival rate; reducing the risk of recurrence of the cancer; reducing the risk of progression; and/or increasing the chance of progression-free survival, relapse-free survival, and/or recurrence-free survival.

123. The method of any one of claims 116-122, wherein the ratio of (i) to (ii) is 1:1.

124. The method of any one of claims 116-123, wherein (i) and (ii) are administered within 30 days of each other.

125. The method of any one of claim 116-124, wherein (i) and (ii) are administered on the same day.

126. The method of any one of claims 116-125, wherein the T cells of (i) comprise a TCR that has been isolated and cloned from T cells that are stimulated in response to subject-derived B cells comprising an expression vector that encodes at least one minigene linked to a promoter and wherein the minigene encodes a neoantigen.

127. The method of any one of claims 116-126, wherein the T cells of (ii) comprise a TCR that has been isolated and cloned from T cells that are stimulated in response to lysates of subject-derived B cells comprising an expression vector that encodes at least one minigene linked to a promoter and wherein the minigene encodes a neoantigen.

128. The method of any one of claims 116-127, wherein about 1×10⁷ cells of (i) and about 1×10⁷ cells of (ii) are administered to the subject.

129. The method of any one of claims 116-128, wherein the CD4+ and/or CD8+ T cells comprise a convergent CDR3-alpha and/or CDR3-beta.

130. A method for treating a subject with a T cell receptor (TCR), the method comprising administering a TCR to the subject, wherein the TCR comprises a convergent CDR3-alpha and/or CDR3-beta.

131. The method of claim 130, wherein the amino acid sequence of the alpha chain CDR1, CDR2, and CDR3 and beta chain CDR1, CDR2, and CDR3 of the administered TCR comprises the amino acid sequence of an alpha chain CDR1, CDR2, and CDR3 and beta chain CDR1, CDR2, and CDR3 from a TCR comprising a convergent CDR3-alpha and/or CDR3-beta that has been isolated and sequenced from cells from the subject.

132. A method comprising:
isolating TCRs from a subject;
sequencing the isolated TCRs;
administering to the subject an engineered TCR comprising an alpha chain CDR1, CDR2, and CDR3 and a beta chain CDR1, CDR2, and CDR3 of a sequenced TCR;
wherein the engineered TCR comprises a convergent CDR3-alpha and/or CDR3-beta.

133. The method of any one of claims 130-132, wherein administering an engineered TCR comprises administering T cells comprising a heterologous nucleic acid encoding for the engineered TCR.

134. The method of claim 133, wherein the T cells comprise autologous T cells.

135. The method of claim 133 or 134, wherein the T cells comprise CD4+ or CD8+ T cells.

136. The method of any one of claims 130-135, wherein a convergent CDR3-alpha or a convergent CDR3 beta comprises an amino acid sequence that is identical in at least three different TCR clones isolated and sequenced in cells from the subject.

137. The method of claim 136, wherein the at least three different TCR clones comprise an identical CDR3-alpha and/or CDR3-beta amino acid sequence and a different nucleotide sequence.

138. The method of claim 137, wherein the nucleotide sequence differs at the V-J joint of the CDR3-alpha, the V-D joint of the CDR3-beta, and/or the D-J joint of the CDR3-beta.

139. The method of claim 136, wherein at least three different TCR clones comprise an identical CDR3-alpha and/or CDR3-beta amino acid and nucleotide sequence and wherein the V(D)J region haplotypes that are utilized to generate the CDR3 are different in the at least three different TCR clones.

140. The method of any one of claims 132-139, wherein sequencing the isolated TCRs comprises single cell sequencing of nucleic acids isolated from T cells isolated from the subject.

141. The method of claim 140, wherein the TCR-beta chain is sequenced.

142. The method of claim 140, wherein the TCR-alpha chain is sequenced.

143. The method of claim 140, wherein the TCR-alpha and TCR-beta chain is sequenced.

144. The method of any one of claims 140-143, wherein the T cells isolated from the subject comprise CD3+, CD8+, and/or CD4+ T cells.

145. The method of any one of claims 130-144, wherein the frequency of the CDR3-alpha and/or CDR3-beta is greater than 0.01.

146. The method of any one of claims 130-145, wherein the subject has been treated with a cancer vaccine.

147. The method of any one of claims 130-145, wherein the subject has not been treated with a cancer vaccine.

148. The method of any one of claims 130-147, wherein the subject has not previously been treated with an immunotherapy.

149. The method of claim 148, wherein the immunotherapy comprises immune checkpoint blockade (ICB) therapy.

150. The method of claim 148, wherein the immunotherapy comprises adoptive T cell therapy, a tumor cell vaccine, or a dendritic cell vaccine.


SEQUENCE LISTING (53 sequences)

SEQ ID NO: 1 - protein, 261 aa, synthetic construct
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 2 - protein, 15 aa, synthetic construct
GLNDIFEAQKIEWHE

SEQ ID NO: 3 - protein, 26 aa, synthetic construct
KRRWKKNFIAVSAANRFKKISSSGAL

SEQ ID NO: 4 - protein, 6 aa, synthetic construct
EEEEEE

SEQ ID NO: 5 - protein, 13 aa, synthetic construct
GAPVPYPDPLEPR

SEQ ID NO: 6 - protein, 8 aa, synthetic construct
DYKDDDDK

SEQ ID NO: 7 - protein, 9 aa, synthetic construct
YPYDVPDYA

SEQ ID NO: 8 - protein, 6 aa, synthetic construct
HHHHHH

SEQ ID NO: 9 - protein, 10 aa, synthetic construct
EQKLISEEDL

SEQ ID NO: 10 - protein, 18 aa, synthetic construct
TKENPRSNQEESYDDNES

SEQ ID NO: 11 - protein, 15 aa, synthetic construct
KETAAAKFERQHMDS

SEQ ID NO: 12 - protein, 38 aa, synthetic construct
MDEKTTGWRGGHVVEGLAGELEQLRARLEHHPQGQREP

SEQ ID NO: 13 - protein, 13 aa, synthetic construct
SLAELLNAGLGGS

SEQ ID NO: 14 - protein, 8 aa, synthetic construct
TQDPSRVG

SEQ ID NO: 15 - protein, 8 aa, synthetic construct
WSHPQFEK

SEQ ID NO: 16 - protein, 6 aa, synthetic construct
CCPGCC

SEQ ID NO: 17 - protein, 14 aa, synthetic construct
GKPIPNPLLGLDST

SEQ ID NO: 18 - protein, 11 aa, synthetic construct
YTDIEMNRLGK

SEQ ID NO: 19 - protein, 8 aa, synthetic construct
DLYDDDDK

SEQ ID NO: 20 - protein, 12 aa, synthetic construct
KLGDIEFIKVNK

SEQ ID NO: 21 - protein, 16 aa, synthetic construct
TDKDMTITFTNKKDAE

SEQ ID NO: 22 - protein, 13 aa, synthetic construct
AHIVMVDAYKPTK

SEQ ID NO: 23 - protein, 24 aa, synthetic construct
CAAGYGGSGNKLIFGTGTLLSVKP

SEQ ID NO: 24 - protein, 25 aa, synthetic construct
CASSIGTGGNERLFFGHGTKLSVLE

SEQ ID NO: 25 - protein, 24 aa, synthetic construct
CASATSSGQKLVFGQGTILKVYLH

SEQ ID NO: 26 - protein, 26 aa, synthetic construct
CASGDAGTGGSYEQYFGPGTRLTVLE

SEQ ID NO: 27 - protein, 25 aa, synthetic construct
CAAERTSSGQKLVFGQGTILKVYLH

SEQ ID NO: 28 - protein, 25 aa, synthetic construct
CASSIGTGGNERLFFGHGTKLSVLE

SEQ ID NO: 29 - protein, 24 aa, synthetic construct
CAVSGGSNYKLTFGKGTLLTVTPN

SEQ ID NO: 30 - protein, 24 aa, synthetic construct
CAWSLWGGYAEQFFGPGTRLTVLE

SEQ ID NO: 31 - protein, 15 aa, synthetic construct
GCTGCTGGTTATGGG

SEQ ID NO: 32 - protein, 30 aa, synthetic construct
AGCAGTATTGGGACAGGGGGCAACGAAAGA

SEQ ID NO: 33 - protein, 15 aa, synthetic construct
GCTGCTGGGTATGGG

SEQ ID NO: 34 - protein, 30 aa, synthetic construct
AGCAGTATCGGGACAGGGGGCAACGAAAGA

SEQ ID NO: 35 - protein, 30 aa, synthetic construct
AGCAGTATCGGGACAGGGGGGAACGAAAGA

SEQ ID NO: 36 - protein, 30 aa, synthetic construct
AGCAGTATAGGGACAGGGGGCAACGAAAGA

SEQ ID NO: 37 - protein, 30 aa, synthetic construct
AGCAGTATCGGGACAGGGGGTAACGAAAGA

SEQ ID NO: 38 - protein, 30 aa, synthetic construct
AGCAGTATCGGGACAGGGGGAAACGAAAGA

SEQ ID NO: 39 - protein, 15 aa, synthetic construct
GCTGCTGGCTATGGG

SEQ ID NO: 40 - protein, 15 aa, synthetic construct
GCTTCCGCAACTTCA

SEQ ID NO: 41 - protein, 30 aa, synthetic construct
GGTGATGCAGGGACTGGGGGGTCCTATGAA

SEQ ID NO: 42 - protein, 30 aa, synthetic construct
GGTGATGCCGGGACAGGGGGCTCCTATGAA

SEQ ID NO: 43 - protein, 30 aa, synthetic construct
GGTGATGCCGGGACTGGGGGCTCCTATGAA

SEQ ID NO: 44 - protein, 30 aa, synthetic construct
GGTGATGCCGGGACAGGGGGTTCCTATGAA

SEQ ID NO: 45 - protein, 30 aa, synthetic construct
GGTGATGCTGGGACAGGGGGTTCCTATGAA

SEQ ID NO: 46 - protein, 30 aa, synthetic construct
GGTGATGCCGGGACAGGGGGATCCTATGAA

SEQ ID NO: 47 - protein, 15 aa, synthetic construct
GCTGTGAGCGGAGGC

SEQ ID NO: 48 - protein, 15 aa, synthetic construct
GCTGTGAGTGGAGGC

SEQ ID NO: 49 - protein, 30 aa, synthetic construct
TGGAGTCTCTGGGGGGGCTATGCTGAGCAG

SEQ ID NO: 50 - protein, 30 aa, synthetic construct
TGGAGTCTATGGGGGGGCTATGCTGAGCAG

SEQ ID NO: 51 - protein, 30 aa, synthetic construct
TGGAGTCTGTGGGGGGGCTATGCTGAGCAG

SEQ ID NO: 52 - protein, 30 aa, synthetic construct
TGGAGTCTCTGGGGGGGATATGCTGAGCAG

SEQ ID NO: 53 - protein, 30 aa, synthetic construct
TGGAGTCTCTGGGGGGGGTATGCTGAGCAG